(19) United States
(12) Patent Application Publication
(10) Pub. No.: US 2016/0168171 A1
(43) Pub. Date: Jun. 16, 2016

(54) METHODS OF PRODUCING ORGANOSILICA MATERIALS AND USES THEREOF

(71) Applicants: Quanchang Li, Dayton, NJ (US); Mobae Afeworki, Phillipsburg, NJ (US); David Charles Calabro, Bridgewater, NJ (US); David Griffin, Phillipsburg, NJ (US); Meghan Nines, Lansdale, PA (US); Simon Christopher Weston, Annandale, NJ (US); Paul Podsiadlo, Easton, PA (US); Jean Willem Lodewijk Beeckman, Columbia, MD (US); Preeti Kamakoti, Summit, NJ (US); Kanmi Mao, Clinton, NJ (US); Matu J. Shah, Hackettstown, NJ (US)

(72) Inventors: Quanchang Li, Dayton, NJ (US); Mobae Afeworki, Phillipsburg, NJ (US); David Charles Calabro, Bridgewater, NJ (US); David Griffin, Phillipsburg, NJ (US); Meghan Nines, Lansdale, PA (US); Simon Christopher Weston, Annandale, NJ (US); Paul Podsiadlo, Easton, PA (US); Jean Willem Lodewijk Beeckman, Columbia, MD (US); Preeti Kamakoti, Summit, NJ (US); Kanmi Mao, Clinton, NJ (US); Matu J. Shah, Hackettstown, NJ (US)

(73) Assignee: ExxonMobil Research and Engineering Company, Annandale, NJ (US)

(21) Appl. No.: 14/966,001

(22) Filed: Dec. 11, 2015

Related U.S. Application Data
(60) Provisional application No. 62/091,077, filed on Dec. 12, 2014.
(60) Provisional application No. 62/091,071, filed on Dec. 12, 2014.

(51) Int. Cl. C07F 7/08; B01J 20/28; B01J 20/22; B01J 31/02; B01J 35/10

(52) U.S. Cl. CPC C07F 7/0807; B01J 31/0274; B01J 35/1061; B01J 35/1019; B01J 35/1023; B01J 35/1028; B01J 20/22; B01J 20/28083; B01J 20/28061; B01J 20/28064; B01J 20/28066

(57) ABSTRACT

Methods of preparing organosilica materials, which is a polymer comprising independent siloxane units of Formula [Z³Z⁴SiCH₂]₃ (I), wherein each Z³ represents a hydroxyl group, a C₁-C₄ alkoxy group or an oxygen atom bonded to a silicon atom of another siloxane unit and each Z⁴ represents a hydroxyl group, a C₁-C₄ alkoxy group, a C₁-C₄ alkyl group, or an oxygen atom bonded to a silicon atom of another siloxane, in the absence of a structure directing agent and/or porogen are provided herein. Processes of using the organosilica materials, e.g., for gas separation, etc., are also provided herein.

### CROSS-REFERENCE TO RELATED APPLICATIONS

This application claims the benefit of provisional U.S. Ser. No. 62/091,077 and provisional U.S. Ser. No. 62/091,071, filed Dec. 12, 2014, the entire contents of which are expressly incorporated by reference herein.

This application is also related to several other co-pending U.S. applications, filed on even date herewith and bearing Attorney Docket Nos. 2014EM304-US2 (entitled “Organosilica Materials and Uses Thereof”), 2015EM382 (entitled “Aromatic Hydrogenation Catalysts and Uses Thereof”), 2015EM383 (entitled “Organosilica Materials and Uses Thereof”), 2015EM384 (entitled “Organosilica Materials and Uses Thereof”), 2015EM385 (entitled “Organosilica Materials and Uses Thereof”), 2015EM386 (entitled “Organosilica Materials and Uses Thereof”), 2015EM387 (entitled “Coating Method Using Organosilica Materials and Uses Thereof”), 2015EM388 (entitled “Membrane Fabrication Method Using Organosilica Materials and Uses Thereof”), 2015EM389 (entitled “Adsorbent for Heteroatom Species Removal and Uses Thereof”), and 2015EM390 (entitled “Method for Separating Aromatic Compounds from Lube Basestocks”), the entire disclosures of each of which are incorporated by reference herein.

Additionally, this application is further related to several other co-pending U.S. applications, filed on even date herewith and bearing Attorney Docket Nos. 2015EM375 (entitled “Organosilica Materials for Use as Adsorbents for Oxygenate Removal”), 2015EM376 (entitled “Supported Catalyst for Olefin Polymerization”), 2015EM377 (entitled “Supported Catalyst for Olefin Polymerization”), 2015EM378 (entitled “Supported Catalyst for Olefin Polymerization”), and 2015EM379 (entitled “Supported Catalyst for Olefin Polymerization”), the entire disclosures of each of which are incorporated by reference herein.

### FIELD OF THE INVENTION

The present invention relates to a method of producing organosilica materials.

### BACKGROUND OF THE INVENTION

Porous inorganic solids have found great utility as catalysts and separation media for industrial application. In particular, mesoporous materials, such as silicas and aluminas, having a periodic arrangement of mesopores are attractive materials for use in adsorption, separation and catalysis processes due to their uniform and tunable pores, high surface areas and large pore volumes. The pore structure of such mesoporous materials is large enough to absorb large molecules and the pore wall structure can be as thin as about 1 nm. Further, such mesoporous materials are known to have large specific surface areas (e.g., 1000 m²/g) and large pore volumes (e.g., 1 cm³/g). For these reasons, such mesoporous materials enable reactive catalysts, adsorbents composed of a functional organic compound, and other molecules to rapidly diffuse into the pores and therefore, can be advantageous over zeolites, which have smaller pore sizes. Consequently, such mesoporous materials can be useful not only for catalysis of high-speed catalytic reactions, but also as large capacity adsorbents.

It was further discovered that the inclusion of some organic groups in the mesoporous framework can provide adjustable reactive surfaces and also contributes to uniformity in pore size, higher mechanical strength, and hydrothermal stability of the material. Thus, mesoporous organosilica materials can exhibit unique properties compared to mesoporous silica such as enhanced hydrothermal stability, chemical stability, and mechanical properties. Organic groups can be incorporated using bridged silsesquioxane precursors of the form Si—R—Si to form mesoporous organosilicas.

Mesoporous organosilicas are conventionally formed by the self-assembly of the silsequioxane precursor in the presence of a structure directing agent, a porogen and/or a framework element. The precursor is hydrolysable and condenses around the structure directing agent. These materials have been referred to as Periodic Mesoporous Organosilicates (PMOs), due to the presence of periodic arrays of parallel aligned mesoscale channels. For example, Landskron, K., et al. [Science, 302:266-269 (2003)] report the self-assembly of 1,3,5-tris[diethoxysila]cylcohexane [(EtO)₂SiCH₂]₃ in the presence of a base and the structure directing agent, cetyltrimethylammonium bromide to form PMOs that are bridged organosilicas with a periodic mesoporous framework, which consist of SiO₃R or SiO₂R₂ building blocks, where R is a bridging organic group. In PMOs, the organic groups can be homogenously distributed in the pore walls. U.S. Pat. Pub. No. 2012/0059181 reports the preparation of a crystalline hybrid organic-inorganic silicate formed from 1,1,3,3,5,5 hexaethoxy-1,3,5 trisilyl cyclohexane in the presence of NaAlO₂ and base. U.S. Patent Application Publication No. 2007/003492 reports preparation of a composition formed from 1,1,3,3,5,5 hexaethoxy-1,3,5 trisilyl cyclohexane in the presence of propylene glycol monomethyl ether.

However, the use of a structure directing agent, such as a surfactant, in the preparation of an organosilica material, such as a PMO, requires a complicated, energy intensive process to eliminate the structure directing agent at the end of the preparation process. This limits the ability to scale-up the process for industrial applications. Therefore, there is a need to provide a method for preparing organosilica materials with a desirable pore diameter, pore volume and surface area, by a method that can be practiced in the absence of a structure directing agent, a porogen or surfactant.

### SUMMARY OF THE INVENTION

It has been found that an organosilica material can be successfully prepared with desirable pore diameter, pore volume, and surface area without the need for a structure directing agent, a porogen or surfactant.

Thus, in one aspect, embodiments of the invention provide a method for preparing an organosilica material, the method comprising: (a) providing an aqueous mixture that contains essentially no structure directing agent and/or porogen, (b) adding at least one compound of Formula [Z¹Z²SiCH₂]₃ (Ia) into the aqueous mixture to form a solution, wherein each Z¹ represents a C₁-C₄ alkoxy group and each Z² represents a C₁-C₄ alkoxy group or a C₁-C₄ alkyl group; (c) aging the solution to produce a pre-product (e.g., a gel); and (d) drying the pre-product (e.g., a gel) to obtain an organosilica material which is a polymer comprising siloxane units of Formula [Z³Z⁴SiCH₂]₃ (I), wherein each Z³ represents a hydroxyl group, a C₁-C₄ alkoxy group or an oxygen atom bonded to a silicon atom of another siloxane and each Z⁴ represents a hydroxyl group, a C₁-C₄ alkoxy group, a C₁-C₄ alkyl group, or an oxygen atom bonded to a silicon atom of another siloxane.

In still another aspect, embodiments of the invention provide an organosilica material made according to the methods described herein.

In still another aspect, embodiments of the invention provide a catalyst material made comprising the organosilica material described herein and optionally, a binder.

In still another aspect, embodiments of the invention provide a method for preparing an organosilica material, the method comprising: (a) adding a compound corresponding in structure to Formula (Ib)

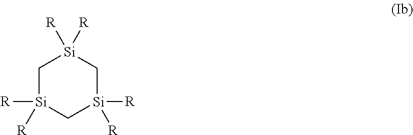

wherein each R is independently selected from the group consisting of a C₁-C₂ alkoxy and a C₁-C₂ alkyl into an aqueous mixture to form a solution; (b) aging the solution to produce a gel; and (c) drying the gel to obtain the organosilica material having an X-ray diffraction spectrum exhibiting substantially no peaks above 6 degrees 2θ; and wherein the method is performed using substantially no structure directing agent.

Other embodiments, including particular aspects of the embodiments summarized above, will be evident from the detailed description that follows.

### BRIEF DESCRIPTION OF THE DRAWINGS

FIG. 1 illustrates an X-Ray Diffraction (XRD) spectrum for Sample 1A and Comparative Sample 2.

FIG. 2a illustrates thermal gravimetric analysis (TGA) data for Sample 1A in N₂.

FIG. 2b illustrates TGA data for Sample 1A in air.

FIG. 3 illustrates the nitrogen adsorption/desorption analysis for Sample 1A, Comparative Sample 2 and Sample 5.

FIG. 4 illustrates a BET pore diameter distribution for Sample 1A, Comparative Sample 2 and Sample 5.

FIG. 5 illustrates comparison of BET surface area and microporous surface area for Sample 1A, Sample 3, Sample 5A and Sample 6.

FIG. 6 illustrates comparison of pore volume and pore diameter for Sample 1A, Sample 3, Sample 5A and Sample 6.

FIG. 7a illustrates a ²⁹Si MAS NMR spectrum for Sample 1A.

FIG. 7b illustrates a ²⁹Si MAS NMR spectrum for Comparative Sample 2.

FIG. 8a illustrates TGA data for Comparative Sample 2 in N₂.

FIG. 8b illustrates TGA data for Comparative Sample 2 in air.

FIG. 9 illustrates an XRD spectrum for Sample 1A and Sample 3.

FIG. 10 illustrates a ²⁹Si MAS NMR spectrum for Sample 4A, Sample 4B, Sample 4D and Sample 4E.

FIG. 11 illustrates an XRD spectrum for Sample 5 and Sample 6.

FIG. 12 illustrates TGA data for Sample 5 in air and N₂.

FIG. 13 illustrates a ²⁹Si MAS NMR spectrum for Sample 1A and Sample 5.

FIG. 14 illustrates a ²⁹Si MAS NMR spectrum for Sample 7A and Sample 7B.

FIG. 15 illustrates an XRD spectrum for Sample 9, Sample 10, Sample 11A, and Sample 12.

FIG. 16 illustrates an XRD spectrum for Sample 13 and Sample 21.

FIG. 17 illustrates N₂ adsorption isotherms for Sample 13, Sample 14 and Sample 15.

FIG. 18 illustrates a BET pore diameter distribution for Sample 13, Sample 14 and Sample 15.

FIG. 19 illustrates an XRD spectrum for Sample 22A and Sample 22B.

FIG. 20 illustrates a ²⁹Si MAS NMR spectrum for Sample 22A and Sample 22B.

FIG. 21 illustrates a ²⁹Al MAS NMR spectrum for Sample 22A and Sample 22B.

FIG. 22a illustrates BET surface area and microporous surface area for samples made with varying pHs.

FIG. 22b illustrates pore volume and average pore radius for samples made with varying pHs.

FIG. 23a illustrates N₂ adsorption isotherms for samples with varying aging times.

FIG. 23b illustrates BET surface area and microporous surface area for samples with varying aging times.

FIG. 24a illustrates pore diameter distribution for samples with varying aging times.

FIG. 24b illustrates pore volume and average pore radius for samples with varying aging times.

FIG. 25a illustrates BET surface area for samples with varying aging times at an aging temperature of 120° C.

FIG. 25b illustrates pore volume and average pore diameter for samples with varying aging times at an aging temperature of 120° C.

FIG. 26 illustrates a ²⁹Si MAS NMR spectrum for samples with varying aging times and aging temperatures.

FIG. 27 illustrates a ¹³C MAS NMR spectrum for samples with varying aging times and aging temperatures.

FIG. 28 illustrates CO₂ adsorption isotherms for Sample 1A, Sample 5 and Comparative Sample 2.

FIG. 29 illustrates an XRD spectrum for Sample 1A, Sample 1A(i), Sample 1A(ii), Sample 1A(iii), and Sample 1A(iv).

FIG. 30 illustrates carbon content change for Sample 1A, Sample 1A(i), Sample 1A(ii), Sample 1A(iii), and Sample 1A(iv).

FIG. 31 illustrates BET surface area change for Sample 1A, Sample 1A(i), Sample 1A(ii), Sample 1A(iii), and Sample 1A(iv).

FIG. 32 illustrates pore volume and average pore diameter change of Sample 1A, Sample 1A(i), Sample 1A(ii), Sample 1A(iii), and Sample 1A(iv).

### DETAILED DESCRIPTION OF THE INVENTION

In various aspects of the invention, organosilica materials, methods for preparing organosilica materials and gas and liquid separation processes using the organosilica materials are provided.

### I. DEFINITIONS

For purposes of this invention and the claims hereto, the numbering scheme for the Periodic Table Groups is according to the IUPAC Periodic Table of Elements.

The term “and/or” as used in a phrase such as “A and/or B” herein is intended to include “A and B”, “A or B”, “A”, and “B”.

The terms “substituent”, “radical”, “group”, and “moiety” may be used interchangeably.

As used herein, and unless otherwise specified, the term “Cₑ” means hydrocarbon(s) having n carbon atom(s) per molecule, wherein n is a positive integer.

As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n.

As used herein, and unless otherwise specified, the term “alkyl” refers to a saturated hydrocarbon radical having from 1 to 12 carbon atoms (i.e. C₁-C₁₂ alkyl), particularly from 1 to 8 carbon atoms (i.e. C₁-C₈ alkyl), particularly from 1 to 6 carbon atoms (i.e. C₁-C₆ alkyl), and particularly from 1 to 4 carbon atoms (i.e. C₁-C₄ alkyl). Examples of alkyl groups include, but are not limited to, methyl, ethyl, propyl, butyl, pentyl, hexyl, heptyl, octyl, decyl, and so forth. The alkyl group may be linear, branched or cyclic. “Alkyl” is intended to embrace all structural isomeric forms of an alkyl group. For example, as used herein, propyl encompasses both n-propyl and isopropyl; butyl encompasses n-butyl, sec-butyl, isobutyl and tert-butyl and so forth. As used herein, “C₁ alkyl” refers to methyl (—CH₃), “C₂ alkyl” refers to ethyl (—CH₂CH₃), “C₃ alkyl” refers to propyl (—CH₂CH₂CH₃) and “C₄ alkyl” refers to butyl (e.g. —CH₂CH₂CH₂CH₃, —(CH₃)CHCH₂CH₃, —CH₂CH(CH₃)₂, etc.). Further, as used herein, “Me” refers to methyl, and “Et” refers to ethyl, “i-Pr” refers to isopropyl, “t-Bu” refers to tert-butyl, and “Np” refers to neopentyl.

As used herein, and unless otherwise specified, the term “alkylene” refers to a divalent alkyl moiety containing 1 to 12 carbon atoms (i.e. C₁-C₁₂ alkylene) in length and meaning the alkylene moiety is attached to the rest of the molecule at both ends of the alkyl unit. For example, alkylenes include, but are not limited to, —CH₂—, —CH₂CH₂—, —CH(CH₃)CH₂—, —CH₂CH₂CH₂—, etc. The alkylene group may be linear or branched.

As used herein, and unless otherwise specified, the term “nitrogen-containing alkyl” refers to an alkyl group as defined herein wherein one or more carbon atoms in the alkyl group is substituted with a nitrogen atom or a nitrogen-containing cyclic hydrocarbon having from 2 to 10 carbon atoms (i.e., a nitrogen-containing cyclic C₂-C₁₀ hydrocarbon), particularly having from 2 to 5 carbon atoms (i.e., a nitrogen-containing cyclic C₂-C₅ hydrocarbon), and particularly having from 2 to 5 carbon atoms (i.e., a nitrogen-containing cyclic C₂-C₅ hydrocarbon). The nitrogen-containing cyclic hydrocarbon may have one or more nitrogen atoms. The nitrogen atom(s) may optionally be substituted with one or two C₁-C₆ alkyl groups. The nitrogen-containing alkyl can have from 1 to 12 carbon atoms (i.e. C₁-C₁₂ nitrogen-containing alkyl), particularly from 1 to 10 carbon atoms (i.e. C₁-C₁₀ nitrogen-containing alkyl), particularly from 2 to 10 carbon atoms (i.e. C₂-C₁₀ nitrogen-containing alkyl), particularly from 3 to 10 carbon atoms (i.e. C₃-C₁₀ nitrogen-containing alkyl), and particularly from 3 to 8 carbon atoms (i.e. C₁-C₁₀ nitrogen-containing alkyl). Examples of nitrogen-containing alkyls include, but are not limited to,

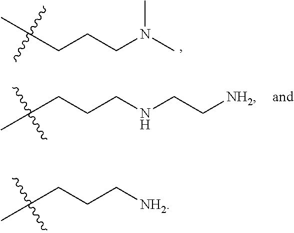

As used herein, and unless otherwise specified, the term “nitrogen-containing alkylene” refers to an alkylene group as defined herein wherein one or more carbon atoms in the alkyl group is substituted with a nitrogen atom. The nitrogen atom(s) may optionally be substituted with one or two C₁-C₆ alkyl groups. The nitrogen-containing alkylene can have from 1 to 12 carbon atoms (i.e. C₁-C₁₂ nitrogen-containing alkylene), particularly from 2 to 10 carbon atoms (i.e. C₂-C₁₀ nitrogen-containing alkylene), particularly from 3 to 10 carbon atoms (i.e. C₃-C₁₀ nitrogen-containing alkylene), particularly from 4 to 10 carbon atoms (i.e. C₄-C₁₀ nitrogen-containing alkylene), and particularly from 3 to 8 carbon atoms (i.e. C₃-C₈ nitrogen-containing alkyl). Examples of nitrogen-containing alkylenes include, but are not limited to,

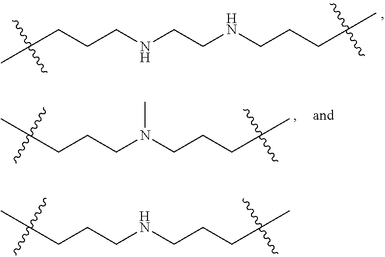

As used herein, and unless otherwise specified, the term “alkenyl” refers to an unsaturated hydrocarbon radical having from 2 to 12 carbon atoms (i.e., C₂-C₁₂ alkenyl), particularly from 2 to 8 carbon atoms (i.e., C₂-C₈ alkenyl), particularly from 2 to 6 carbon atoms (i.e., C₂-C₆ alkenyl), and having one or more (e.g., 2, 3, etc.) carbon-carbon double bonds. The alkenyl group may be linear, branched or cyclic. Examples of alkenyls include, but are not limited to ethenyl (vinyl), 2-propenyl, 3-propenyl, 1,4-pentadienyl, 1,4-butadienyl, 1-butenyl, 2-butenyl and 3-butenyl. “Alkenyl” is intended to embrace all structural isomeric forms of an alkenyl. For example, butenyl encompasses 1,4-butadienyl, 1-butenyl, 2-butenyl and 3-butenyl, etc.

As used herein, and unless otherwise specified, the term “alkenylene” refers to a divalent alkenyl moiety containing 2 to about 12 carbon atoms (i.e. C₂-C₁₂ alkenylene) in length and meaning that the alkylene moiety is attached to the rest of the molecule at both ends of the alkyl unit. For example, alkenylenes include, but are not limited to, —CH═CH—, —CH═CHCH₂—, —CH═CH═CH—, —CH₂CH₂CH═CHCH₂—, etc. —CH₂CH₂—, —CH(CH₃)CH₂—, —CH₂CH₂CH₂—, etc. The alkenylene group may be linear or branched.

As used herein, and unless otherwise specified, the term “alkynyl” refers to an unsaturated hydrocarbon radical having from 2 to 12 carbon atoms (i.e., C₂-C₁₂ alkynyl), particularly from 2 to 8 carbon atoms (i.e., C₂-C₈ alkynyl), particularly from 2 to 6 carbon atoms (i.e., C₂-C₆ alkynyl), and having one or more (e.g., 2, 3, etc.) carbon-carbon triple bonds. The alkynyl group may be linear, branched or cyclic. Examples of alkynyls include, but are not limited to ethynyl, 1-propynyl, 2-butynyl, and 1,3-butadiynyl. “Alkynyl” is intended to embrace all structural isomeric forms of an alkynyl. For example, butynyl encompasses 2-butynyl, and 1,3-butadiynyl and propynyl encompasses 1-propynyl and 2-propynyl (propargyl).

As used herein, and unless otherwise specified, the term “alkynylene” refers to a divalent alkynyl moiety containing 2 to about 12 carbon atoms (i.e. C₂-C₁₂ alkenylene) in length and meaning that the alkylene moiety is attached to the rest of the molecule at both ends of the alkyl unit. For example, alkenylenes include, but are not limited to, —C≡C—, —C≡CCH₂—, —C≡CCH₂C≡C—, —CH₂CH₂C≡CCH₂—, etc. —CH₂CH₂—, —CH(CH₃)CH₂—, —CH₂CH₂CH₂—, etc. The alkynlene group may be linear or branched.

As used herein, and unless otherwise specified, the term “alkoxy” refers to —O—-alkyl containing from 1 to about 10 carbon atoms. The alkoxy may be straight-chain or branched-chain. Non-limiting examples include methoxy, ethoxy, propoxy, butoxy, isobutoxy, tert-butoxy, pentoxy, and hexoxy. “C₁ alkoxy” refers to methoxy, “C₂ alkoxy” refers to ethoxy, “C₃ alkoxy” refers to propoxy and “C₄ alkoxy” refers to butoxy. Further, as used herein, “OMe” refers to methoxy and “OEt” refers to ethoxy.

As used herein, and unless otherwise specified, the term “aromatic” refers to unsaturated cyclic hydrocarbons having a delocalized conjugated π system and having from 5 to 20 carbon atoms (aromatic C₅-C₂₀ hydrocarbon), particularly from 5 to 12 carbon atoms (aromatic C₅-C₁₂ hydrocarbon), and particularly from 5 to 10 carbon atoms (aromatic C₅-C₁₂ hydrocarbon). Exemplary aromatics include, but are not limited to benzene, toluene, xylenes, mesitylene, ethylbenzenes, cumene, naphthalene, methylnaphthalene, dimethylnaphthalenes, ethylnaphthalenes, acenaphthalene, anthracene, phenanthrene, tetraphene, naphthacene, benzanthracenes, fluoranthrene, pyrene, chrysene, triphenylene, and the like, and combinations thereof. Additionally, the aromatic may comprise one or more heteroatoms. Examples of heteroatoms include, but are not limited to, nitrogen, oxygen, and/or sulfur. Aromatics with one or more heteroatom include, but are not limited to furan, benzofuran, thiophene, benzothiophene, oxazole, thiazole and the like, and combinations thereof. The aromatic may comprise monocyclic, bicyclic, tricyclic, and/or polycyclic rings (in some embodiments, at least monocyclic rings, only monocyclic and bicyclic rings, or only monocyclic rings) and may be fused rings.

As used herein, and unless otherwise specified, the term “aryl” refers to any monocyclic or polycyclic cyclized carbon radical containing 6 to 14 carbon ring atoms, wherein at least one ring is an aromatic hydrocarbon. Examples of aryls include, but are not limited to phenyl, naphthyl, pyridinyl, and indolyl.

As used herein, and unless otherwise specified, the term “aralkyl” refers to an alkyl group substituted with an aryl group. The alkyl group may be a C₁-C₁₀ alkyl group, particularly a C₁-C₆, particularly a C₁-C₄ alkyl group, and particularly a C₁-C₃ alkyl group. Examples of aralkyl groups include, but are not limited to phenymethyl, phenylethyl, and naphthylmethyl. The aralkyl may comprise one or more heteroatoms and be referred to as a “heteroaralkyl.” Examples of heteroatoms include, but are not limited to, nitrogen (i.e., nitrogen-containing heteroaralkyl), oxygen (i.e., oxygen-containing heteroaralkyl), and/or sulfur (i.e., sulfur-containing heteroaralkyl). Examples of heteroaralkyl groups include, but are not limited to, pyridinylethyl, indolylmethyl, furylethyl, and quinolinylpropyl.

As used herein, and unless otherwise specified, the term “heterocyclo” refers to fully saturated, partially saturated or unsaturated or polycyclic cyclized carbon radical containing from 4 to 20 carbon ring atoms and containing one or more heteroatoms atoms. Examples of heteroatoms include, but are not limited to, nitrogen (i.e., nitrogen-containing heterocyclo), oxygen (i.e., oxygen-containing heterocyclo), and/or sulfur (i.e., sulfur-containing heterocyclo). Examples of heterocyclo groups include, but are not limited to, thienyl, furyl, pyrrolyl, piperazinyl, pyridyl, benzoxazolyl, quinolinyl, imidazolyl, pyrrolidinyl, and piperidinyl.

As used herein, and unless otherwise specified, the term “heterocycloalkyl” refers to an alkyl group substituted with heterocyclo group. The alkyl group may be a C₁-C₁₀ alkyl group, particularly a C₁-C₆, particularly a C₁-C₄ alkyl group, and particularly a C₁-C₃ alkyl group. Examples of heterocycloalkyl groups include, but are not limited to thienylmethyl, furylethyl, pyrrolylmethyl, piperazinylethyl, pyridylmethyl, benzoxazolylethyl, quinolinylpropyl, and imidazolylpropyl.

As used herein, the term “hydroxyl” refers to an —OH group.

As used herein, the term “mesoporous” refers to solid materials having pores that have a diameter within the range of from about 2 nm to about 50 nm.

As used herein, the term “organosilica” refers to an organosiloxane compound that comprises one or more organic groups bound to two or more Si atoms.

As used herein, the term “silanol” refers to a Si—OH group.

As used herein, the term “silanol content” refers to the percent of the Si—OH groups in a compound and can be calculated by standard methods, such as NMR.

As used herein, the terms “structure directing agent,” “SDA,” and/or “porogen” refer to one or more compounds added to the synthesis media to aid in and/or guide the polymerization and/or polycondensing and/or organization of the building blocks that form the organosilica material framework. Further, a “porogen” is understood to be a compound capable of forming voids or pores in the resultant organosilica material framework. As used herein, the term “structure directing agent” encompasses and is synonymous and interchangeable with the terms “templating agent” and “template.”

As used herein, and unless otherwise specified, the term “adsorption” includes physisorption, chemisorption, and condensation onto a solid material and combinations thereof.

### II. METHODS OF PRODUCING ORGANOSILICA MATERIAL

The invention relates to methods of producing an organosilica material. In a first embodiment, the method comprises:

(a) providing an aqueous mixture that contains essentially no structure directing agent and/or porogen;

(b) adding at least one compound of Formula [Z¹Z²SiCH₂]₃ (Ia) into the aqueous mixture to form a solution, wherein each Z¹ represents a C₁-C₄ alkoxy group and each Z² represents a C₁-C₄ alkoxy group or a C₁-C₄ alkyl group;

(c) aging the solution to produce a pre-product; and

(d) drying the pre-product to obtain an organosilica material which is a polymer comprising siloxane units of Formula [Z³Z⁴SiCH₂]₃ (I), wherein each Z³ represents a hydroxyl group, a C₁-C₄ alkoxy group or an oxygen atom bonded to a silicon atom of another siloxane and each Z⁴ represents a hydroxyl group, a C₁-C₄ alkoxy group, a C₁-C₄ alkyl group, or an oxygen atom bonded to a silicon atom of another siloxane.

As used herein, and unless otherwise specified, “oxygen atom bonded to a silicon atom of another siloxane” means that the oxygen atom can advantageously displace a moiety (particularly an oxygen-containing moiety such as a hydroxyl, an alkoxy or the like), if present, on a silicon atom of another siloxane so the oxygen atom may be bonded directly to the silicon atom of another siloxane thereby connecting the two siloxanes, e.g., via a Si—O—Si linkage. For clarity, in this bonding scenario, the “another siloxane” can be a siloxane of the same type or a siloxane of a different type.

Additionally or alternatively, the at least one compound of Formula [Z¹Z²SiCH₂]₃ (Ia) can be added in step (b) as at least partially hydroxylated and/or as at least partially polymerized/oligomerized, such that each Z¹ can more broadly represent a hydroxyl group, a C alkoxy group or an oxygen atom bonded to a silicon atom of another siloxane and each Z² can more broadly represent a hydroxyl group, a C₁-C₄ alkoxy group, a C₁-C₄ alkyl group, or an oxygen atom bonded to a silicon atom of another siloxane. In other words, an unaged pre-product can be added in step (b), in addition to or as an alternative to the monomeric (at least one) compound of Formula [Z¹Z²SiCH₂]₃ (Ia).

II.A. Aqueous Mixture

The aqueous mixture contains essentially no added structure directing agent and/or no added porogen.

As used herein, “no added structure directing agent,” and “no added porogen” means either (i) there is no component present in the synthesis of the organosilica material that aids in and/or guides the polymerization and/or polycondensing and/or organization of the building blocks that form the framework of the organosilica material; or (ii) such component is present in the synthesis of the organosilica material in a minor, or a non-substantial, or a negligible amount such that the component cannot be said to aid in and/or guide the polymerization and/or polycondensing and/or organization of the building blocks that form the framework of the organosilica material. Further, “no added structure directing agent” is synonymous with “no added template” and “no added templating agent.”

1. Structure Directing Agent

Examples of a structure directing agent can include, but are not limited to, non-ionic surfactants, ionic surfactants, cationic surfactants, silicon surfactants, amphoteric surfactants, polyalkylene oxide surfactants, fluorosurfactants, colloidal crystals, polymers, hyper branched molecules, star-shaped molecules, macromolecules, dendrimers, and combinations thereof. Additionally or alternatively, the surface directing agent can comprise or be a poloxamer, a triblock polymer, a tetraalkylammonium salt, a nonionic polyoxyethylene alkyl, a Gemini surfactant, or a mixture thereof. Examples of a tetraalkylammonium salt can include, but are not limited to, cetyltrimethylammonium halides, such as cetyltrimethylammonium chloride (CTAC), cetyltrimethylammonium bromide (CTAB), and octadecyltrimethylammonium chloride. Other exemplary surface directing agents can additionally or alternatively include hexadecyltrimethylammonium chloride and/or cetylpyridinium bromide.

Poloxamers are block copolymers of ethylene oxide and propylene oxide, more particularly nonionic triblock copolymers composed of a central hydrophobic chain of polyoxypropylene (poly(propylene oxide)) flanked by two hydrophilic chains of polyoxyethylene (poly(ethylene oxide)). Specifically, the term “poloxamer” refers to a polymer having the formula HO(C₂H₄))a(C₃H₆O)_{b}(C₂H₄O)ₐH in which “a” and “b” denote the number of polyoxyethylene and polyoxypropylene units, respectively. Poloxamers are also known by the trade name Pluronic®, for example Pluronic® 123 and Pluronic® F127. An additional triblock polymer is B50-6600.

Nonionic polyoxyethylene alkyl ethers are known by the trade name Brij®, for example Brij® 56, Brij® 58, Brij® 76, Brij® 78. Gemini surfactants are compounds having at least two hydrophobic groups and at least one or optionally two hydrophilic groups per molecule have been introduced.

2. Porogen

A porogen material is capable of forming domains, discrete regions, voids and/or pores in the organosilica material. As used herein, porogen does not include water. An example of a porogen is a block copolymer (e.g., a di-block polymer). Examples of polymer porogens can include, but are not limited to, polyvinyl aromatics, such as polystyrenes, polyvinylpyridines, hydrogenated polyvinyl aromatics, polyacrylonitriles, polyalkylene oxides, such as polyethylene oxides and polypropylene oxides, polyethylenes, polylactic acids, polysiloxanes, polycaprolactones, polycaprolactams, polyurethanes, polymethacrylates, such as polymethylmethacrylate or polymethacrylic acid, polyacrylates, such as polymethylacrylate and polyacrylic acid, polydienes such as polybutadienes and polyisoprenes, polyvinyl chlorides, polyacetals, and amine-capped alkylene oxides, as well as combinations thereof.

Additionally or alternatively, porogens can be thermoplastic homopolymers and random (as opposed to block) copolymers. As used herein, “homopolymer” means compounds comprising repeating units from a single monomer. Suitable thermoplastic materials can include, but are not limited to, homopolymers or copolymers of polystyrenes, polyacrylates, polymethacrylates, polybutadienes, polyisoprenes, polyphenylene oxides, polypropylene oxides, polyethylene oxides, poly(dimethylsiloxanes), polytetrahydrofurans, polyethylenes, polycyclohexylethylenes, polyethyloxazolines, polyvinylpyridines, polycaprolactones, polylactic acids, copolymers of these materials and mixtures of these materials. Examples of polystyrene include, but are not limited to anionic polymerized polystyrene, syndiotactic polystyrene, unsubstituted and substituted polystyrenes (for example, poly(α-methyl styrene)). The thermoplastic materials may be linear, branched, hyperbranched, dendritic, or star like in nature.

Additionally or alternatively, the porogen can be a solvent. Examples of solvents can include, but are not limited to, ketones (e.g., cyclohexanone, cyclopentanone, 2-heptanone, cycloheptanone, cyclooctanone, cyclohexylpyrrolidinone, methyl isobutyl ketone, methyl ethyl ketone, acetone), carbonate compounds (e.g., ethylene carbonate, propylene carbonate), heterocyclic compounds (e.g., 3-methyl-2-oxazolidinone, dimethylimidazolidinone, N-methylpyrrolidone, pyridine), cyclic ethers (e.g., dioxane, tetrahydrofuran), chain ethers (e.g., diethyl ether, ethylene glycol dimethyl ether, propylene glycol dimethyl ether, tetraethylene glycol dimethyl ether, polyethylene glycol dimethyl ether, ethylene glycol monomethyl ether, ethylene glycol monoethyl ether, propylene glycol monomethyl ether (PGME), triethylene glycol monobutyl ether, propylene glycol monopropyl ether, triethylene glycol monomethyl ether, diethylene glycol ethyl ether, diethylene glycol methyl ether, dipropylene glycol methyl ether, dipropylene glycol dimethyl ether, propylene glycol phenyl ether, tripropylene glycol methyl ether), alcohols (e.g., methanol, ethanol), polyhydric alcohols (e.g., ethylene glycol, propylene glycol, polyethylene glycol, polypropylene glycol, glycerin, dipropylene glycol), nitrile compounds (e.g., acetonitrile, glutarodinitrile, methoxyacetonitrile, propionitrile, benzonitrile), esters (e.g., ethyl acetate, butyl acetate, methyl lactate, ethyl lactate, methyl methoxypropionate, ethyl ethoxypropionate, methyl pyruvate, ethyl pyruvate, propyl pyruvate, 2-methoxyethyl acetate, ethylene glycol monoethyl ether acetate, propylene glycol monomethyl ether acetate (PGMEA), butyrolactone, phosphoric acid ester, phosphonic acid ester), aprotic polar substances (e.g., dimethyl sulfoxide, sulfolane, dimethylformamide, dimethylacetamide), nonpolar solvents (e.g., toluene, xylene, mesitylene), chlorine-based solvents (e.g., methylene dichloride, ethylene dichloride), benzene, dichlorobenzene, naphthalene, diphenyl ether, diisopropylbenzene, triethylamine, methyl benzoate, ethyl benzoate, butyl benzoate, monomethyl ether acetate hydroxy ethers such as dibenzylethers, diglyme, triglyme, and mixtures thereof.

3. Base/Acid

In various embodiments, the aqueous mixture used in methods provided herein can comprise a base and/or an acid.

In certain embodiments where the aqueous mixture comprises a base, the aqueous mixture can have a pH from about 8 to about 14, from about 8 to about 13.5, from about 8 to about 13, from about 8 to about 12.5, from about 8 to about 12, from about 8 to about 11.5, from about 8 to about 11, from about 8 to about 10.5, from about 8 to about 10, from about 8 to about 9.5, from about 8 to about 9, from about 8 to about 8.5, from about 8.5 to about 15, from about 8.5 to about 14.5, from about 8.5 to about 14, from about 8.5 to about 13.5, from about 8.5 to about 13, from about 8.5 to about 12.5, from about 8.5 to about 12, from about 8.5 to about 11.5, from about 8.5 to about 11, from about 8.5 to about 10.5, from about 8.5 to about 10, from about 8.5 to about 9.5, from about 8.5 to about 9, from about 9 to about 15, from about 9 to about 14.5, from about 9 to about 14, from about 9 to about 13.5, from about 9 to about 13, from about 9 to about 12.5, from about 9 to about 12, from about 9 to about 11.5, from about 9 to about 11, from about 9 to about 10.5, from about 9 to about 10, from about 9 to about 9.5, from about 9.5 to about 15, from about 9.5 to about 14.5, from about 9.5 to about 14, from about 9.5 to about 13.5, from about 9.5 to about 13, from about 9.5 to about 12.5, from about 9.5 to about 12, from about 9.5 to about 11.5, from about 9.5 to about 11, from about 9.5 to about 10.5, from about 9.5 to about 10, from about 10 to about 15, from about 10 to about 14.5, from about 10 to about 14, from about 10 to about 13.5, from about 10 to about 13, from about 10 to about 12.5, from about 10 to about 12, from about 10 to about 11.5, from about 10 to about 11, from about 10 to about 10.5, from about 10.5 to about 15, from about 10.5 to about 14.5, from about 10.5 to about 14, from about 10.5 to about 13.5, from about 10.5 to about 13, from about 10.5 to about 12.5, from about 10.5 to about 12, from about 10.5 to about 11.5, from about 10.5 to about 11, from about 11 to about 15, from about 11 to about 14.5, from about 11 to about 14, from about 11 to about 13.5, from about 11 to about 13, from about 11 to about 12.5, from about 11 to about 12, from about 11 to about 11.5, from about 11.5 to about 15, from about 11.5 to about 14.5, from about 11.5 to about 14, from about 11.5 to about 13.5, from about 11.5 to about 13, from about 11.5 to about 12.5, from about 11.5 to about 12, from about 12 to about 15, from about 12 to about 14.5, from about 12 to about 14, from about 12 to about 13.5, from about 12 to about 13, from about 12 to about 12.5, from about 12.5 to about 15, from about 12.5 to about 14.5, from about 12.5 to about 14, from about 12.5 to about 13.5, from about 12.5 to about 13, from about 12.5 to about 15, from about 12.5 to about 14.5, from about 12.5 to about 14, from about 12.5 to about 13.5, from about 12.5 to about 13, from about 13 to about 15, from about 13 to about 14.5, from about 13 to about 14, from about 13 to about 13.5, from about 13.5 to about 15, from about 13.5 to about 14.5, from about 13.5 to about 14, from about 14 to about 15, from about 14 to about 14.5, and from about 14.5 to about 15.

In a particular embodiment comprising a base, the pH can be from about 9 to about 15, from about 9 to about 14 or from about 8 to about 14.

Exemplary bases can include, but are not limited to, sodium hydroxide, potassium hydroxide, lithium hydroxide, pyridine, pyrrole, piperazine, pyrrolidine, piperidine, picoline, monoethanolamine, diethanolamine, dimethylmonoethanolamine, monomethyldiethanolamine, triethanolamine, diazabicyclooctane, diazabicyclononane, diazabicycloundecene, tetramethylammonium hydroxide, tetraethylammonium hydroxide, tetrapropylammonium hydroxide, tetrabutylammonium hydroxide, ammonia, ammonium hydroxide, methylamine, ethylamine, propylamine, butylamine, pentylamine, hexylamine, octylamine, nonylamine, decylamine, N,N-dimethylamine, N,N-diethylamine, N,N-dipropylamine, N,N-dibutylamine, trimethylamine, triethylamine, tripropylamine, tributylamine, cyclohexylamine, trimethylimidine, 1-amino-3-methylbutane, dimethylglycine, 3-amino-3-methylamine, and the like. These bases may be used either singly or in combination. In a particular embodiment, the base can comprise or be sodium hydroxide and/or ammonium hydroxide.

In certain embodiments where the aqueous mixture comprises an acid, the aqueous mixture can have a pH from about 0.01 to about 6.0, from about 0.01 to about 5, from about 0.01 to about 4, from about 0.01 to about 3, from about 0.01 to about 2, from about 0.01 to about 1, from about 0.1 to about 6.0, about 0.1 to about 5.5, about 0.1 to about 5.0, from about 0.1 to about 4.8, from about 0.1 to about 4.5, from about 0.1 to about 4.2, from about 0.1 to about 4.0, from about 0.1 to about 3.8, from about 0.1 to about 3.5, from about 0.1 to about 3.2, from about 0.1 to about 3.0, from about 0.1 to about 2.8, from about 0.1 to about 2.5, from about 0.1 to about 2.2, from about 0.1 to about 2.0, from about 0.1 to about 1.8, from about 0.1 to about 1.5, from about 0.1 to about 1.2, from about 0.1 to about 1.0, from about 0.1 to about 0.8, from about 0.1 to about 0.5, from about 0.1 to about 0.2, about 0.2 to about 6.0, about 0.2 to about 5.5, from about 0.2 to about 5, from about 0.2 to about 4.8, from about 0.2 to about 4.5, from about 0.2 to about 4.2, from about 0.2 to about 4.0, from about 0.2 to about 3.8, from about 0.2 to about 3.5, from about 0.2 to about 3.2, from about 0.2 to about 3.0, from about 0.2 to about 2.8, from about 0.2 to about 2.5, from about 0.2 to about 2.2, from about 0.2 to about 2.0, from about 0.2 to about 1.8, from about 0.2 to about 1.5, from about 0.2 to about 1.2, from about 0.2 to about 1.0, from about 0.2 to about 0.8, from about 0.2 to about 0.5, about 0.5 to about 6.0, about 0.5 to about 5.5, from about 0.5 to about 5, from about 0.5 to about 4.8, from about 0.5 to about 4.5, from about 0.5 to about 4.2, from about 0.5 to about 4.0, from about 0.5 to about 3.8, from about 0.5 to about 3.5, from about 0.5 to about 3.2, from about 0.5 to about 3.0, from about 0.5 to about 2.8, from about 0.5 to about 2.5, from about 0.5 to about 2.2, from about 0.5 to about 2.0, from about 0.5 to about 1.8, from about 0.5 to about 1.5, from about 0.5 to about 1.2, from about 0.5 to about 1.0, from about 0.5 to about 0.8, about 0.8 to about 6.0, about 0.8 to about 5.5, from about 0.8 to about 5, from about 0.8 to about 4.8, from about 0.8 to about 4.5, from about 0.8 to about 4.2, from about 0.8 to about 4.0, from about 0.8 to about 3.8, from about 0.8 to about 3.5, from about 0.8 to about 3.2, from about 0.8 to about 3.0, from about 0.8 to about 2.8, from about 0.8 to about 2.5, from about 0.8 to about 2.2, from about 0.8 to about 2.0, from about 0.8 to about 1.8, from about 0.8 to about 1.5, from about 0.8 to about 1.2, from about 0.8 to about 1.0, about 1.0 to about 6.0, about 1.0 to about 5.5, from about 1.0 to about 5.0, from about 1.0 to about 4.8, from about 1.0 to about 4.5, from about 1.0 to about 4.2, from about 1.0 to about 4.0, from about 1.0 to about 3.8, from about 1.0 to about 3.5, from about 1.0 to about 3.2, from about 1.0 to about 3.0, from about 1.0 to about 2.8, from about 1.0 to about 2.5, from about 1.0 to about 2.2, from about 1.0 to about 2.0, from about 1.0 to about 1.8, from about 1.0 to about 1.5, from about 1.0 to about 1.2, about 1.2 to about 6.0, about 1.2 to about 5.5, from about 1.2 to about 5.0, from about 1.2 to about 4.8, from about 1.2 to about 4.5, from about 1.2 to about 4.2, from about 1.2 to about 4.0, from about 1.2 to about 3.8, from about 1.2 to about 3.5, from about 1.2 to about 3.2, from about 1.2 to about 3.0, from about 1.2 to about 2.8, from about 1.2 to about 2.5, from about 1.2 to about 2.2, from about 1.2 to about 2.0, from about 1.2 to about 1.8, from about 1.2 to about 1.5, about 1.5 to about 6.0, about 1.5 to about 5.5, from about 1.5 to about 5.0, from about 1.5 to about 4.8, from about 1.5 to about 4.5, from about 1.5 to about 4.2, from about 1.5 to about 4.0, from about 1.5 to about 3.8, from about 1.5 to about 3.5, from about 1.5 to about 3.2, from about 1.5 to about 3.0, from about 1.5 to about 2.8, from about 1.5 to about 2.5, from about 1.5 to about 2.2, from about 1.5 to about 2.0, from about 1.5 to about 1.8, about 1.8 to about 6.0, about 1.8 to about 5.5, from about 1.8 to about 5.0, from about 1.8 to about 4.8, from about 1.8 to about 4.5, from about 1.8 to about 4.2, from about 1.8 to about 4.0, from about 1.8 to about 3.8, from about 1.8 to about 3.5, from about 1.8 to about 3.2, from about 1.8 to about 3.0, from about 1.8 to about 2.8, from about 1.8 to about 2.5, from about 1.8 to about 2.2, from about 1.8 to about 2.0, about 2.0 to about 6.0, about 2.0 to about 5.5, from about 2.0 to about 5.0, from about 2.0 to about 4.8, from about 2.0 to about 4.5, from about 2.0 to about 4.2, from about 2.0 to about 4.0, from about 2.0 to about 3.8, from about 2.0 to about 3.5, from about 2.0 to about 3.2, from about 2.0 to about 3.0, from about 2.0 to about 2.8, from about 2.0 to about 2.5, from about 2.0 to about 2.2, about 2.2 to about 6.0, about 2.2 to about 5.5, from about 2.2 to about 5.0, from about 2.2 to about 4.8, from about 2.2 to about 4.5, from about 2.2 to about 4.2, from about 2.2 to about 4.0, from about 2.2 to about 3.8, from about 2.2 to about 3.5, from about 2.2 to about 3.2, from about 2.2 to about 3.0, from about 2.2 to about 2.8, from about 2.2 to about 2.5, about 2.5 to about 6.0, about 2.5 to about 5.5, from about 2.5 to about 5.0, from about 2.5 to about 4.8, from about 2.5 to about 4.5, from about 2.5 to about 4.2, from about 2.5 to about 4.0, from about 2.5 to about 3.8, from about 2.5 to about 3.5, from about 2.5 to about 3.2, from about 2.5 to about 3.0, from about 2.5 to about 2.8, from about 2.8 to about 6.0, about 2.8 to about 5.5, from about 2.8 to about 5.0, from about 2.8 to about 4.8, from about 2.8 to about 4.5, from about 2.8 to about 4.2, from about 2.8 to about 4.0, from about 2.8 to about 3.8, from about 2.8 to about 3.5, from about 2.8 to about 3.2, from about 2.8 to about 3.0, from about 3.0 to about 6.0, from about 3.5 to about 5.5, from about 3.0 to about 5.0, from about 3.0 to about 4.8, from about 3.0 to about 4.5, from about 3.0 to about 4.2, from about 3.0 to about 4.0, from about 3.0 to about 3.8, from about 3.0 to about 3.5, from about 3.0 to about 3.2, from about 3.2 to about 6.0, from about 3.2 to about 5.5, from about 3.2 to about 5, from about 3.2 to about 4.8, from about 3.2 to about 4.5, from about 3.2 to about 4.2, from about 3.2 to about 4.0, from about 3.2 to about 3.8, from about 3.2 to about 3.5, from about 3.5 to about 6.0, from about 3.5 to about 5.5, from about 3.5 to about 5, from about 3.5 to about 4.8, from about 3.5 to about 4.5, from about 3.5 to about 4.2, from about 3.5 to about 4.0, from about 3.5 to about 3.8, from about 3.8 to about 5, from about 3.8 to about 4.8, from about 3.8 to about 4.5, from about 3.8 to about 4.2, from about 3.8 to about 4.0, from about 4.0 to about 6.0, from about 4.0 to about 5.5, from about 4.0 to about 5, from about 4.0 to about 4.8, from about 4.0 to about 4.5, from about 4.0 to about 4.2, from about 4.2 to about 5, from about 4.2 to about 4.8, from about 4.2 to about 4.5, from about 4.5 to about 5, from about 4.5 to about 4.8, or from about 4.8 to about 5.

In a particular embodiment comprising an acid, the pH can be from about 0.01 to about 6.0, about 0.2 to about 6.0, about 0.2 to about 5.0 or about 0.2 to about 4.5.

Exemplary acids can include, but are not limited to, inorganic acids such as hydrochloric acid, nitric acid, sulfuric acid, hydrofluoric acid, phosphoric acid, boric acid and oxalic acid; and organic acids such as acetic acid, propionic acid, butanoic acid, pentanoic acid, hexanoic acid, heptanoic acid, octanoic acid, nonanoic acid, decanoic acid, oxalic acid, maleic acid, methylmalonic acid, adipic acid, sebacic acid, gallic acid, butyric acid, mellitic acid, arachidonic acid, shikimic acid, 2-ethylhexanoic acid, oleic acid, stearic acid, linoleic acid, linolenic acid, salicylic acid, benzoic acid, p-amino-benzoic acid, p-toluenesulfonic acid, benzenesulfonic acid, monochloroacetic acid, dichloroacetic acid, trichloroacetic acid, trifluoroacetic acid, formic acid, malonic acid, sulfonic acid, phthalic acid, fumaric acid, citric acid, tartaric acid, succinic acid, itaconic acid, mesaconic acid, citraconic acid, malic acid, a hydrolysate of glutaric acid, a hydrolysate of maleic anhydride, a hydrolysate of phthalic anhydride, and the like. These acids may be used either singly or in combination. In a particular embodiment, the acid can comprise or be hydrochloric acid.

In various aspects, adjusting the pH of the aqueous mixture can affect the total surface area, microporous surface area and pore volume of the organosilica material made. Thus, the porosity of the organosilica material may be adjusted by adjusting the pH of the aqueous mixture.

For example, when the aqueous mixture is basic and has a pH between about 8 to about 14, in particular about 9 to about 14, the organosilica material made may have one or more of the following characteristics:
(i) a total surface area of about 200 m²/g to about 1800 m²/g, particularly about 300 m²/g to about 1700 m²/g, and particularly about 400 m²/g to about 1700 m²/g;
(ii) a microporous surface area of about 0 m²/g to about 700 m²/g, and particularly about 0 m²/g to about 700 m²/g;
(iii) a pore volume of about 0.2 cm³/g to about 3 cm³/g, and particularly of about 0.8 cm³/g to about 1.4 cm³/g.

Additionally or alternatively, when the aqueous mixture is acidic and has a pH between about 0.1 to about 7, particularly about 0.1 to about 5, particularly about 0.1 to about 4.5, the organosilica material made may have one or more of the following characteristics:
(iv) a total surface area of about 100 m²/g to about 1500 m²/g, particularly about 100 m²/g to about 900 m²/g, and particularly about 200 m²/g to about 900 m²/g;
(v) a microporous surface area of about 100 m²/g to about 600 m²/g, and particularly about 0 m²/g to about 500 m²/g;
(vi) a pore volume of about 0.1 cm³/g to about 1.2 cm³/g, and particularly of about 0.1 cm³/g to about 0.6 cm³/g.

Thus, the total surface area of an organosilica material made with a basic aqueous mixture may increase when compared to an organosilica material made with an acidic aqueous mixture. Further, the pore volume of an organosilica material made with a basic aqueous mixture may increase when compared to an organosilica material made with an acidic aqueous mixture. However, the microporous surface area of an organosilica material made with a basic aqueous mixture may decrease when compared to an organosilica material made with an acidic aqueous mixture.

II.B. Compounds of Formula (Ia)

The methods provided herein comprise the step of adding at least one compound of Formula [Z¹Z²SiCH₂]₃ (Ia) into the aqueous mixture to form a solution, wherein each Z¹ can be a C₁-C₄ alkoxy group and each Z² can be a C₁-C₄ alkoxy group or a C₁-C₄ alkyl group.

In one embodiment, each Z¹ can comprise a C₁-C₃ alkoxy or methoxy or ethoxy.

Additionally or alternatively, each Z² can comprise a C₁-C₄ alkoxy, a C₁-C₃ alkoxy or methoxy or ethoxy. Additionally or alternatively, each Z² can comprise methyl, ethyl or propyl, such as a methyl or ethyl.

Additionally or alternatively, each Z¹ can be a C₁-C₂ alkoxy group and each Z² can be a C₁-C₂ alkoxy group or a C₁-C₂ alkyl group

Additionally or alternatively, each Z¹ can be methoxy or ethoxy and each Z² can be methyl or ethyl.

In a particular embodiment, each Z¹ and each Z² can be ethoxy, such that the compound corresponding to Formula (Ia) can be 1,1,3,3,5,5-hexaethoxy-1,3,5-trisilacyclohexane, [(EtO)₂SiCH₂]₃.

In a particular embodiment, each Z¹ can be ethoxy and each Z² can be methyl, such that compound corresponding to Formula (Ia) can be 1,3,5-trimethyl-1,3,5-triethoxy-1,3,5-trisilacyclohexane, [EtOCH₃SiCH₂]₃.

In various aspects, more than one compound of Formula (Ia) (e.g., same or different compound) may be added to the aqueous mixture to form a solution. For example, [(EtO)₂SiCH₂]₃ and [EtOCH₃SiCH₂]₃ may both be added to the aqueous mixture to form a solution.

When more than one compound of Formula (Ia) is used, the respective compounds may be used in a wide variety of molar ratios. For example, if two compounds of Formula (Ia) are used, the molar ratio of each compound may vary from 1:99 to 99:1, such as from 10:90 to 90:10. The use of different compounds of Formula (Ia) allows to tailor the properties of the organosilica materials made by the process of the invention, as will be further explained in the examples and in the section of this specification describing the properties of the organosilicas made by the present processes.

II.C. Compounds of Formula (II)

In additional embodiments, the methods provided herein can further comprise adding to the aqueous solution a compound of Formula R¹OR²R³R⁴Si (II), wherein each R¹ can be a hydrogen atom or a C₁-C₆ alkyl group, and R², R³ and R⁴ each independently can be selected from the group consisting of a hydrogen atom, a C₁-C₆ alkyl group, a C₁-C₆ alkoxy group, a nitrogen-containing C₁-C₁₀ alkyl group, a nitrogen-containing heteroaralkyl group, and a nitrogen-containing optionally substituted heterocycloalkyl group.

In one embodiment, each R¹ can be a C₁-C₅ alkyl group, a C₁-C₄ alkyl group, a C₁-C₃ alkyl group, a C₁-C₂ alkyl group, or methyl. In particular, each R¹ can be methyl or ethyl.

Additionally or alternatively, R², R³ and R⁴ can be each independently a C₁-C₅ alkyl group, a C₁-C₄ alkyl group, a C₁-C₃ alkyl group, a C₁-C₂ alkyl group, or methyl.

Additionally or alternatively, each R¹ can be a C₁-C₂ alkyl group and R², R³ and R⁴ can be each independently a C₁-C₂ alkyl group.

Additionally or alternatively, R², R³ and R⁴ can be each independently a C₁-C₅ alkoxy group, a C₁-C₄ alkoxy group, a C₁-C₃ alkoxy group, a C₁-C₂ alkoxy group, or methoxy.

Additionally or alternatively, each R¹ can be a C₁-C₂ alkyl group and R², R³ and R⁴ can be each independently a C₁-C₂ alkoxy group.

Additionally or alternatively, each R¹ can be a C₁-C₂ alkyl group and R², R³ and R⁴ can be each independently a C₁-C₂ alkyl group or a C₁-C₂ alkoxy group.

Additionally or alternatively, R², R³ and R⁴ can be each independently a nitrogen-containing C₁-C₉ alkyl group, a nitrogen-containing C₁-C₈ alkyl group, a nitrogen-containing C₁-C₇ alkyl group, a nitrogen-containing C₁-C₆ alkyl group, a nitrogen-containing C₁-C₅ alkyl group, a nitrogen-containing C₁-C₄ alkyl group, a nitrogen-containing C₁-C₃ alkyl group, a nitrogen-containing C₁-C₂ alkyl group, or a methylamine. In particular, R², R³ and R⁴ can be each independently a nitrogen-containing C₂-C₁₀ alkyl group, a nitrogen-containing C₃-C₁₀ alkyl group, a nitrogen-containing C₃-C₉ alkyl group, or a nitrogen-containing C₃-C₈ alkyl group. The aforementioned nitrogen-containing alkyl groups may have one or more nitrogen atoms (e.g., 2, 3, etc.). Examples of nitrogen-containing C₁-C₁₀ alkyl groups include, but are not limited to,

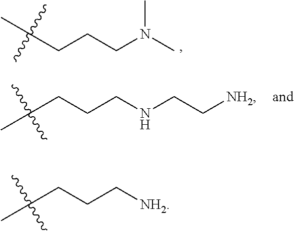

Additionally or alternatively, each R¹ can be a C₁-C₂ alkyl group and R², R³ and R⁴ can be each independently a nitrogen-containing C₃-C₈ alkyl group.

Additionally or alternatively, each R¹ can be a C₁-C₂ alkyl group and R², R³ and R⁴ can be each independently a C₁-C₂ alkyl group, a C₁-C₂ alkoxy group or a nitrogen-containing C₃-C₈ alkyl group.

Additionally or alternatively, R², R³ and R⁴ can be each independently a nitrogen-containing heteroaralkyl group. The nitrogen-containing heteroaralkyl group can be a nitrogen-containing C₄-C₁₂ heteroaralkyl group, a nitrogen-containing C₄-C₁₀ heteroaralkyl group, or a nitrogen-containing C₄-C₈ heteroaralkyl group. Examples of nitrogen-containing heteroaralkyl groups include but are not limited to pyridinylethyl, pyridinylpropyl, pyridinylmethyl, indolylmethyl, pyrazinylethyl, and pyrazinylpropyl. The aforementioned nitrogen-containing heteroaralkyl groups may have one or more nitrogen atoms (e.g., 2, 3, etc.).

Additionally or alternatively, each R¹ can be a C₁-C₂ alkyl group and R², R³ and R⁴ can be each independently a nitrogen-containing heteroaralkyl group.

Additionally or alternatively, each R¹ can be a C₁-C₂ alkyl group and R², R³ and R⁴ can be each independently a C₁-C₂ alkyl group, a C₁-C₂ alkoxy group, a nitrogen-containing C₃-C₈ alkyl group or a nitrogen-containing heteroaralkyl group.

Additionally or alternatively, R², R³ and R⁴ can be each independently a nitrogen-containing heterocycloalkyl group, wherein the heterocycloalkyl group may be optionally substituted with a C₁-C₆ alkyl group, particularly a C₁-C₄ alkyl group. The nitrogen-containing heterocycloalkyl group can be a nitrogen-containing C₄-C₁₂ heterocycloalkyl group, a nitrogen-containing C₄-C₁₀ heterocycloalkyl group, or a nitrogen-containing C₄-C₈ heterocycloalkyl group. Examples of nitrogen-containing heterocycloalkyl groups include but are not limited to piperazinylethyl, piperazinylpropyl, piperidinylethyl, piperidinylpropyl. The aforementioned nitrogen-containing heterocycloalkyl groups may have one or more nitrogen atoms (e.g., 2, 3, etc.).

Additionally or alternatively, each R¹ can be a C₁-C₂ alkyl group and R², R³ and R⁴ can be each independently a nitrogen-containing optionally substituted heterocycloalkyl group.

Additionally or alternatively, each R¹ can be a C₁-C₂ alkyl group and R², R³ and R⁴ can be each independently a C₁-C₂ alkyl group, a C₁-C₂ alkoxy group, a nitrogen-containing C₃-C₈ alkyl group, a nitrogen-containing heteroaralkyl group, or a nitrogen-containing optionally substituted heterocycloalkyl group.

Additionally or alternatively, each R¹ can be a C₁-C₂ alkyl group and R², R³ and R⁴ can be each independently a C₁-C₂ alkyl group, C₁-C₂ alkoxy group, a nitrogen-containing C₃-C₁₀ alkyl group, a nitrogen-containing C₄-C₁₀ heteroaralkyl group, or a nitrogen-containing optionally substituted C₄-C₁₀ heterocycloalkyl group

In a particular embodiment, each R¹ can be ethyl and each R², R³ and R⁴ can be ethoxy, such that the compound corresponding to Formula (II) can be tetraethyl orthosilicate (TEOS) ((EtO)₄Si).

In another particular embodiment, each R¹ can be ethyl, each R² can be methyl and each R³ and R⁴ can be ethoxy, such that the compound corresponding to Formula (II) can be methyltriethoxysilane (MTES) ((EtO)₃CH₃Si).

In another particular embodiment, each R¹ can be ethyl, each R² and R³ can be ethoxy and each R⁴ can be

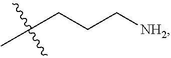 [SMILES: *CCCN]

such that the compound corresponding to Formula (II) can be (3-aminopropyl)triethoxysilane (H₂N(CH₂)₃(EtO)₃Si).

In another particular embodiment, each R¹ can be methyl, each R² and R³ can be methoxy and each R⁴ can be

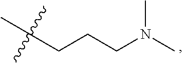 [SMILES: *CCCN(C)C]

such that the compound corresponding to Formula (II) can be (N,N-dimethylaminopropyl)trimethoxysilane (((CH₃)₂N(CH₂)₃)(MeO)₃Si).

In another particular embodiment, each R¹ can be ethyl, each R² and R³ can be ethoxy and each R⁴ can be

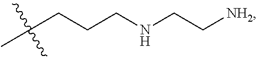 [SMILES: *CCCNCCN]

such that the compound corresponding to Formula (II) can be (N-(2-aminoethyl)-3-aminopropyltriethoxysilane ((H₂N(CH₂)₂NH(CH₂)₃)(EtO)₂Si).

In another particular embodiment, each R¹ can be ethyl, each R² and R³ can be ethoxy and each R⁴ can be

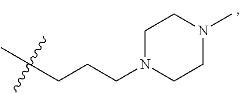 [SMILES: *CCCN1CCN(C)CC1]

such that the compound corresponding to Formula (II) can be 4-methyl-1-(3-triethoxysilylpropyl)-piperazine.

In another particular embodiment, each R¹ can be ethyl, each R² and R³ can be ethoxy and each R⁴ can be

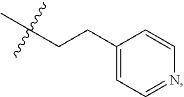 [SMILES: *CCc1ccncc1]

such that the compound corresponding to Formula (II) can be 4-(2-(triethoxysily)ethyl)pyridine.

In another particular embodiment, each R¹ can be ethyl, each R² and R³ can be ethoxy and R⁴ can be

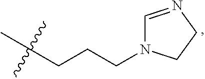 [SMILES: *CCCN1C=NCC1]

such that the compound corresponding to Formula (II) can be 1-(3-(triethoxysilyl)propyl)-4,5-dihydro-1H-imidazole.

The molar ratio of compound of Formula (Ia) to compound of Formula (II) may vary within wide limits, such as from about 99:1 to about 1:99, from about 1:5 to about 5:1, from about 4:1 to about 1:4 or from about 3:2 to about 2:3. For example, a molar ratio of compound of Formula (Ia) to compound of Formula (II) can be from about 4:1 to 1:4 or from about 2.5:1 to about 1:2.5, about 2:1 to about 1:2, such as about 1.5:1 to about 1.5:1.

II.D. Compounds of Formula (III)

In additional embodiments, the methods provided herein can further comprise adding to the aqueous solution a compound of Formula Z⁵Z⁶Z⁷Si—R—Si Z⁵Z⁶Z⁷ (III), wherein each Z⁵ independently can be a C₁-C₄ alkoxy group; each Z⁶ and Z⁷ independently can be a C₁-C₄ alkoxy group or a C₁-C₄ alkyl group; and each R can be selected from the group consisting a C₁-C₈ alkylene group, a C₂-C₈ alkenylene group, a C₂-C₈ alkynylene group, a nitrogen-containing C₁-C₁₀ alkylene group, an optionally substituted C₆-C₂₀ aralkyl group, and an optionally substituted C₄-C₂₀ heterocycloalkyl group.

In one embodiment, each Z⁵ can be a C₁-C₃ alkoxy group, a C₁-C₂ alkoxy group, or methoxy.

Additionally or alternatively, each Z⁶ and Z⁷ independently can be a C₁-C₃ alkoxy group, a C₁-C₂ alkoxy group, or methoxy.

Additionally or alternatively, each Z⁵ can be a C₁-C₂ alkoxy group and each Z⁶ and Z⁷ independently can be a C₁-C₂ alkoxy group.

Additionally or alternatively, each Z⁶ and Z⁷ independently can be a C₁-C₃ alkyl group, a C₁-C₂ alkyl group, or methyl.

Additionally or alternatively, each Z⁵ can be a C₁-C₂ alkoxy group and each Z⁶ and Z⁷ independently can be a C₁-C₂ alkyl group.

Additionally or alternatively, each Z⁵ can be a C₁-C₂ alkoxy group and each Z⁶ and Z⁷ independently can be a C₁-C₂ alkoxy group or a C₁-C₂ alkyl group.

Additionally or alternatively, each R can be a C₁-C₇ alkylene group, a C₁-C₆ alkylene group, a C₁-C₅ alkylene group, a C₁-C₄ alkylene group, a C₁-C₃ alkylene group, a C₁-C₂ alkylene group, or —CH₂—.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₁-C₂ alkylene group.

Additionally or alternatively, each R can be a C₂-C₇ alkenylene group, a C₁-C₆ alkenylene group, a C₂-C₅ alkenylene group, a C₂-C₄ a alkenylene group, a C₂-C₃ alkenylene group, or —CH═CH—.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₁-C₂ alkenylene group.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₁-C₂ alkylene group or a C₁-C₂ alkenylene group.

Additionally or alternatively, each R can be a C₂-C₇ alkynylene group, a C₁-C₆ alkynylene group, a C₂-C₅ alkynylene group, a C₂-C₄ a alkynylene group, a C₂-C₃ alkynylene group, or —C≡C—.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₂-C₄ alkynylene group.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₂-C₄ alkylene group, a C₂-C₄ alkenylene group or a C₂-C₄ alkynylene group.

Additionally or alternatively, each R can be a nitrogen-containing C₂-C₁₀ alkylene group, a nitrogen-containing C₃-C₁₀ alkylene group, a nitrogen-containing C₄-C₁₀ alkylene group, a nitrogen-containing C₄-C₉ alkylene group, a nitrogen-containing C₄-C₈ alkylene group, or nitrogen containing C₃-C₈ alkylene group. The aforementioned nitrogen-containing alkylene groups may have one or more nitrogen atoms (e.g., 2, 3, etc.). Examples of nitrogen-containing alkylene groups include, but are not limited to,

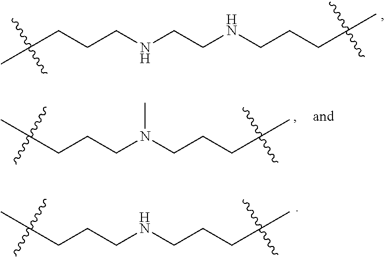

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a nitrogen-containing C₄-C₁₀ alkylene group.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₂-C₄ alkylene group, a C₂-C₄ alkenylene group, a C₂-C₄ alkynylene group or a nitrogen-containing C₄-C₁₀ alkylene group.

Additionally or alternatively, each R can be an optionally substituted C₆-C₂₀ aralkyl, an optionally substituted C₆-C₁₄ aralkyl, or an optionally substituted C₆-C₁₀ aralkyl. Examples of C₆-C₂₀ aralkyls include, but are not limited to, phenymethyl, phenylethyl, and naphthylmethyl. The aralkyl may be optionally substituted with a C₁-C₆ alkyl group, particularly a C₁-C₄ alkyl group.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be an optionally substituted C₆-C₁₀ aralkyl.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₂-C₄ alkylene group, a C₂-C₄ alkenylene group, a C₂-C₄ alkynylene group, a nitrogen-containing C₄-C₁₀ alkylene group, or an optionally substituted C₆-C₁₀ aralkyl.

Additionally or alternatively, each R can be an optionally substituted C₄-C₂₀ heterocycloalkyl group, an optionally substituted C₄-C₁₆ heterocycloalkyl group, an optionally substituted C₄-C₁₂ heterocycloalkyl group, or an optionally substituted C₄-C₁₀ heterocycloalkyl group. Examples of C₄-C₂₀ heterocycloalkyl groups include, but are not limited to, thienylmethyl, furylethyl, pyrrolylmethyl, piperazinylethyl, pyridylmethyl, benzoxazolylethyl, quinolinylpropyl, and imidazolylpropyl. The heterocycloalkyl may be optionally substituted with a C₁-C₆ alkyl group, particularly a C₁-C₄ alkyl group.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be an optionally substituted C₄-C₁₂ heterocycloalkyl group.

Additionally or alternatively, each Z⁵ can be a C₁-C₂ alkoxy group; each Z⁶ and Z⁷ independently can be a C₁-C₂ alkoxy group or a C₁-C₂ alkyl group; and each R can be a C₂-C₄ alkylene group, a C₂-C₄ alkenylene group, a C₂-C₄ alkynylene group, a nitrogen-containing C₄-C₁₀ alkylene group, an optionally substituted C₆-C₁₀ aralkyl, or an optionally substituted C₄-C₁₂ heterocycloalkyl group.

In a particular embodiment, each Z⁵ and Z⁶ can be ethoxy, each Z⁷ can be methyl and each R can be —CH₂CH₂—, such that compound corresponding to Formula (III) can be 1,2-bis(methyldiethoxysilyl)ethane (CH₃(EtO)₂Si—CH₂CH₂—Si(EtO)₂CH₃).

In a particular embodiment, each Z⁵, Z⁶ and Z⁷ can be ethoxy and each R can be —CH₂—, such that compound corresponding to Formula (III) can be bis(triethoxysilyl)methane ((EtO)₃Si—CH₂—Si(EtO)₃).

In a particular embodiment, each Z⁵, Z⁶ and Z⁷ can be ethoxy and each R can be —HC═CH—, such that compound corresponding to Formula (III) can be 1,2-bis(triethoxysilyl)ethylene ((EtO)₃Si—HC═CH—Si(EtO)₃).

In a particular embodiment, each Z⁵, Z⁶ and Z⁷ can be methoxy and each R can be

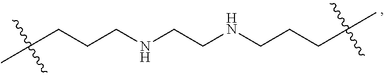 [SMILES: *CCCNCCNCCC*]

such that compound corresponding to Formula (III) can be N,N′-bis[(3-trimethoxysilyl)propyl]ethylenediamine.

In a particular embodiment, each Z⁵ and Z⁶ can be ethoxy, each Z⁷ can be methyl and each R can be

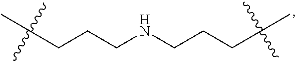 [SMILES: *CCCNCCC*]

such that compound corresponding to Formula (III) can be bis[(methyldiethoxysilyl)propyl]amine.

In a particular embodiment, each Z⁵ and Z⁶ can be methoxy, each Z⁷ can be methyl and each R can be

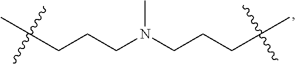 [SMILES: *CCCN(C)CCC*]

such that compound corresponding to Formula (III) can be bis[(methyldimethoxysilyl)propyl]-N-methylamine.

The molar ratio of compound of Formula (Ia) to compound of Formula (III) may vary within wide limits, such as from about 99:1 to about 1:99, from about 1:5 to about 5:1, from about 4:1 to about 1:4 or from about 3:2 to about 2:3. For example, a molar ratio of compound of Formula (Ia) to compound of Formula (III) can be from about 4:1 to 1:4 or from about 2.5:1 to 1:2.5, about 2:1 to about 1:2, such as about 1.5:1 to about 1.5:1.

II.E. Trivalent Metal Oxide Sources

In additional embodiments, the methods provided herein can further comprise adding to the aqueous solution sources of a trivalent metal oxide.

Sources of trivalent metal oxides can include, but are not limited to, corresponding salts, alkoxides, oxides, and/or hydroxides of the trivalent metal, e.g., aluminum sulphate, aluminum nitrate, colloidal alumina, aluminum trihydroxide, hydroxylated alumina, Al₂O₃, aluminum halides (e.g., AlCl₃), NaAlO₂, boron nitride, B₂O₃ and/or H₃BO₃.

In various aspects, the source of trivalent metal oxide may be a compound of formula M¹(OZ⁸)₃ (IV), wherein M¹ can be a Group 13 metal and each Z⁸ independently can be a C₁-C₆ alkyl group.

In one embodiment, M¹ can be B, Al, Ga, In, Il, or Uut. In particular, M¹ can be Al or B.

Additionally or alternatively, each Z⁸ can be a C₁-C₆ alkyl group, a C₁-C₅ alkyl group, a C₁-C₄ alkyl group, a C₁-C₃ alkyl group, a C₁-C₂ alkyl group or methyl. In particular, each Z⁸ can be methyl, ethyl, propyl or butyl.

Additionally or alternatively, M¹ can be Al or B and each Z⁸ can be methyl, ethyl, propyl or butyl.

In a particular embodiment, M¹ can be Al and each Z⁸ can be methyl, such that compound corresponding to Formula (IV) can be aluminum trimethoxide.

In a particular embodiment, M¹ can be Al and each Z⁸ can be ethyl, such that compound corresponding to Formula (IV) can be aluminum triethoxide.

In a particular embodiment, M¹ can be Al and each Z⁸ can be propyl, such that compound corresponding to Formula (IV) can be aluminum isopropoxide.

In a particular embodiment, M¹ can be Al and each Z⁸ can be butyl, such that compound corresponding to Formula (IV) can be aluminum tri-sec-butoxide.

Additionally or alternatively, the source of trivalent metal oxide may be a compound of Formula)(Z⁹O)₂M²-O—Si(OZ¹⁰)₃ (V), wherein M² can be a Group 13 metal and each Z⁹ and Z¹⁰ independently can be a C₁-C₆ alkyl group.

In one embodiment, M² can be B, Al, Ga, In, Il, or Uut. In particular, M¹ can be Al or B.

Additionally or alternatively, each Z⁹ and Z¹⁰ independently can be a C₁-C₆ alkyl group, a C₁-C₅ alkyl group, a C₁-C₄ alkyl group, a C₁-C₃ alkyl group, a C₁-C₂ alkyl group or methyl. In particular, each Z⁹ and Z¹⁰ independently can be methyl, ethyl, propyl or butyl.

Additionally or alternatively, M¹ can be Al or B and each Z⁹ and Z¹⁰ independently can be methyl, ethyl, propyl or butyl.

Additionally or alternatively, the source of a trivalent metal oxide may be a source of a compound of Formula (IV) (e.g., AlCl₃), and/or a source of a compound of Formula (V).

The molar ratio of compound of Formula (Ia) to trivalent metal oxide may vary within wide limits, such as from about 99:1 to about 1:99, from about 30:1 to about 1:1, from about 25:1 to about 1:1, from about 20:1 to about 3:1 or from about 20:1 to about 5:1.

II.f. Molar Ratio

In the methods described herein, a molar ratio of Formula (Ia):Formula (Ia), Formula (Ia):Formula (II), Formula (Ia):Formula (III), Formula (III):Formula (II), Formula (Ia):Formula (IV), and Formula (Ia):Formula (V) of about 99:1 to about 1:99, about 75:1 to about 1:99, about 50:1 to about 1:99, about 25:1 to about 1:99, about 15:1 to about 1:99, about 50:1 to about 1:50, about 25:1 to about 1:25 or about 15:1 to about 1:15 may be used. For example, molar ratios of about 3:2, about 4:1, about 4:3, about 5:1, about 2:3, about 1:1 about 5:2 and about 15:1 may be used. For example, a molar ratio of Formula (Ia):Formula (Ia) can be about 3:2. A molar ratio of Formula (Ia):Formula (II) can be about 2:3, about 4:3, about 4:1 or about 3:2. A molar ratio of Formula (Ia):Formula (III) can be about 2:3, and about 4:1. A molar ratio of Formula (III):Formula (II) can be about 5:2, about 1:1, about 1:2 or about 2:3. A molar ratio of Formula (Ia):Formula (IV) and Formula (Ia):Formula (V) can be about 15:1 or about 5:1.

For the sake of the following discussion, the compounds of Formula (Ia), (Ib), (II) and (III) shall be referred to collectively as starting siloxane. Depending on the choice of starting materials, the solution may have a variety of compositions. For example, if base is used, the solution may have molar ratios of starting siloxane to OFF of from about 1:5 to about 1:20, such as from about 1:5 to about 1:15 or from about 1:5 to 1:10, or from about 1:6 to 1:20. If acid is used, the solution may have molar ratios of starting siloxane:H⁺ of from about 50:1 to about 5:1, such as from about 45:1 to about 10:1. In both cases when acid or base is used, the molar ratios of starting siloxane to H₂O may vary from about 1:50 to about 1:1000, such as from about 1:100 to about 1:500.

II.I. Aging the Solution

The solution formed in the methods described herein can be aged for at least about 4 hours, at least about 6 hours, at least about 12 hours, at least about 18 hours, at least about 24 hours (1 day), at least about 30 hours, at least about 36 hours, at least about 42 hours, at least about 48 hours (2 days), at least about 54 hours, at least about 60 hours, at least about 66 hours, at least about 72 hours (3 days), at least about 96 hours (4 days), at least about 120 hours (5 days) or at least about 144 hours (6 days).

Additionally or alternatively, the solution formed in the methods described herein can be aged for about 4 hours to about 144 hours (6 days), about 4 hours to about 120 hours (5 days), about 4 hours to about 96 hours (4 days), about 4 hours to about 72 hours (3 days), about 4 hours to about 66 hours, about 4 hours to about 60 hours, about 4 hours to about 54 hours, about 4 hours to about 48 hours (2 days), about 4 hours to about 42 hours, about 4 hours to about 36 hours, about 4 hours to about 30 hours, about 4 hours to about 24 hours (1 day), about 4 hours to about 18 hours, about 4 hours to about 12 hours, about 4 hours to about 6 hours, about 6 hours to about 144 hours (6 days), about 6 hours to about 120 hours (5 days), about 6 hours to about 96 hours (4 days), about 6 hours to about 72 hours (3 days), about 6 hours to about 66 hours, about 6 hours to about 60 hours, about 6 hours to about 54 hours, about 6 hours to about 48 hours (2 days), about 6 hours to about 42 hours, about 6 hours to about 36 hours, about 6 hours to about 30 hours, about 6 hours to about 24 hours (1 day), about 6 hours to about 18 hours, about 6 hours to about 12 hours, about 12 hours to about 144 hours (6 days), about 12 hours to about 120 hours (5 days), about 12 hours to about 96 hours (4 days), about 12 hours to about 72 hours (3 days), about 12 hours to about 66 hours, about 12 hours to about 60 hours, about 12 hours to about 54 hours, about 12 hours to about 48 hours (2 days), about 12 hours to about 42 hours, about 12 hours to about 36 hours, about 12 hours to about 30 hours, about 12 hours to about 24 hours (1 day), about 12 hours to about 18 hours, about 18 hours to about 144 hours (6 days), about 18 hours to about 120 hours (5 days), about 18 hours to about 96 hours (4 days), about 18 hours to about 72 hours (3 days), about 18 hours to about 66 hours, about 18 hours to about 60 hours, about 18 hours to about 54 hours, about 18 hours to about 48 hours (2 days), about 18 hours to about 42 hours, about 18 hours to about 36 hours, about 18 hours to about 30 hours, about 18 hours to about 24 hours (1 day), about 24 hours (1 day) to about 144 hours (6 days), about 24 (1 day) hours (1 day) to about 120 hours (5 days), about 24 hours (1 day) to about 96 hours (4 days), about 24 hours (1 day) to about 72 hours (3 days), about 24 hours (1 day) to about 66 hours, about 24 hours (1 day) to about 60 hours, about 24 hours (1 day) to about 54 hours, about 24 hours (1 day) to about 48 hours (2 days), about 24 hours (1 day) to about 42 hours, about 24 hours (1 day) to about 36 hours, about 24 hours (1 day) to about 30 hours, about 30 hours to about 144 hours (6 days), about 30 hours to about 120 hours (5 days), about 30 hours to about 96 hours (4 days), about 30 hours to about 72 hours (3 days), about 30 hours to about 66 hours, about 30 hours to about 60 hours, about 30 hours to about 54 hours, about 30 hours to about 48 hours (2 days), about 30 hours to about 42 hours, about 30 hours to about 36 hours, about 36 hours to about 144 hours (6 days), about 36 hours to about 120 hours (5 days), about 36 hours to about 96 hours (4 days), about 36 hours to about 72 hours (3 days), about 36 hours to about 66 hours, about 36 hours to about 60 hours, about 36 hours to about 54 hours, about 36 hours to about 48 hours (2 days), about 36 hours to about 42 hours, about 42 hours to about 144 hours (6 days), about 42 hours to about 120 hours (5 days), about 42 hours to about 96 hours (4 days), about 42 hours to about 72 hours (3 days), about 42 hours to about 66 hours, about 42 hours to about 60 hours, about 42 hours to about 54 hours, about 42 hours to about 48 hours (2 days), about 48 hours (2 days) to about 144 hours (6 days), about 48 hours (2 days) to about 120 hours (5 days), about 48 hours (2 days) to about 96 hours (4 days), about 48 hours (2 days) to about 72 hours (3 days), about 48 hours (2 days) to about 66 hours, about 48 hours (2 days) to about 60 hours, about 48 hours (2 days) to about 54 hours, about 54 hours to about 144 hours (6 days), about 54 hours to about 120 hours (5 days), about 54 hours to about 96 hours (4 days), about 54 hours to about 72 hours (3 days), about 54 hours to about 66 hours, about 54 hours to about 60 hours, about 60 hours to about 144 hours (6 days), about 60 hours to about 120 hours (5 days), about 60 hours to about 96 hours (4 days), about 60 hours to about 72 hours (3 days), about 60 hours to about 66 hours, about 66 hours to about 144 hours (6 days), about 66 hours to about 120 hours (5 days), about 66 hours to about 96 hours (4 days), about 66 hours to about 72 hours (3 days), about 72 hours (3 days) to about 144 hours (6 days), about 72 hours (3 days) to about 120 hours (5 days), about 72 hours (3 days) to about 96 hours (4 days), about 96 hours (4 days) to about 144 hours (6 days), about 96 hours (4 days) to about 120 hours (5 days), or about 120 hours (5 days) to about 144 hours (6 days).

Additionally or alternatively, the solution formed in the method can be aged at temperature of at least about 10° C., at least about 20° C., at least about 30° C., at least about 40° C., at least about 50° C., at least about 60° C., at least about 70° C., at least about 80° C., at least about 90° C., at least about 100° C., at least about 110° C., at least about 120° C. at least about 130° C., at least about 140° C., at least about 150° C., at least about 175° C., at least about 200° C., at least about 250° C., or about 300° C.

Additionally or alternatively, the solution formed in the method can be aged at temperature of about 10° C. to about 300° C., about 10° C. to about 250° C., about 10° C. to about 200° C., about 10° C. to about 175° C., about 10° C. to about 150° C., about 10° C. to about 140° C., about 10° C. to about 130° C., about 10° C. to about 120° C., about 10° C. to about 110° C., about 10° C. to about 100° C., about 10° C. to about 90° C., about 10° C. to about 80° C., about 10° C. to about 70° C., about 10° C. to about 60° C., about 10° C. to about 50° C., about 20° C. to about 300° C., about 20° C. to about 250° C., about 20° C. to about 200° C., about 20° C. to about 175° C., about 20° C. to about 150° C., about 20° C. to about 140° C., about 20° C. to about 130° C., about 20° C. to about 120° C., about 20° C. to about 110° C., about 20° C. to about 100° C., about 20° C. to about 90° C., about 20° C. to about 80° C., about 20° C. to about 70° C., about 20° C. to about 60° C., about 20° C. to about 50° C., about 30° C. to about 300° C., about 30° C. to about 250° C., about 30° C. to about 200° C., about 30° C. to about 175° C., about 30° C. to about 150° C., about 30° C. to about 140° C., about 30° C. to about 130° C., about 30° C. to about 120° C., about 30° C. to about 110° C., about 30° C. to about 100° C., about 30° C. to about 90° C., about 30° C. to about 80° C., about 30° C. to about 70° C., about 30° C. to about 60° C., about 30° C. to about 50° C., about 50° C. to about 300° C., about 50° C. to about 250° C., about 50° C. to about 200° C., about 50° C. to about 175° C., about 50° C. to about 150° C., about 50° C. to about 140° C., about 50° C. to about 130° C., about 50° C. to about 120° C., about 50° C. to about 110° C., about 50° C. to about 100° C., about 50° C. to about 90° C., about 50° C. to about 80° C., about 50° C. to about 70° C., about 50° C. to about 60° C., about 70° C. to about 300° C., about 70° C. to about 250° C., about 70° C. to about 200° C., about 70° C. to about 175° C., about 70° C. to about 150° C., about 70° C. to about 140° C., about 70° C. to about 130° C., about 70° C. to about 120° C., about 70° C. to about 110° C., about 70° C. to about 100° C., about 70° C. to about 90° C., about 70° C. to about 80° C., about 80° C. to about 300° C., about 80° C. to about 250° C., about 80° C. to about 200° C., about 80° C. to about 175° C., about 80° C. to about 150° C., about 80° C. to about 140° C., about 80° C. to about 130° C., about 80° C. to about 120° C., about 80° C. to about 110° C., about 80° C. to about 100° C., about 80° C. to about 90° C., about 90° C. to about 300° C., about 90° C. to about 250° C., about 90° C. to about 200° C., about 90° C. to about 175° C., about 90° C. to about 150° C., about 90° C. to about 140° C., about 90° C. to about 130° C., about 90° C. to about 120° C., about 90° C. to about 110° C., about 90° C. to about 100° C., about 100° C. to about 300° C., about 100° C. to about 250° C., about 100° C. to about 200° C., about 100° C. to about 175° C., about 100° C. to about 150° C., about 100° C. to about 140° C., about 100° C. to about 130° C., about 100° C. to about 120° C., about 100° C. to about 110° C., about 110° C. to about 300° C., about 110° C. to about 250° C., about 110° C. to about 200° C., about 110° C. to about 175° C., about 110° C. to about 150° C., about 110° C. to about 140° C., about 110° C. to about 130° C., about 110° C. to about 120° C., about 120° C. to about 300° C., about 120° C. to about 250° C., about 120° C. to about 200° C., about 120° C. to about 175° C., about 120° C. to about 150° C., about 120° C. to about 140° C., about 120° C. to about 130° C., about 130° C. to about 300° C., about 130° C. to about 250° C., about 130° C. to about 200° C., about 130° C. to about 175° C., about 130° C. to about 150° C., or about 130° C. to about 140° C.

In various aspects, adjusting the aging time and/or aging temperature of the solution formed in the methods described herein can affect the total surface area, microporous surface area, pore volume, pore radius and pore diameter of the organosilica material made. Thus, the porosity of the organosilica material may be adjusted by adjusting aging time and/or temperature.

For example, when the solution is aged for about 1 hour to about 7 hours (e.g., 1, 2, 3, 4, 5, 6 hours) at a temperature of about 80° C. to about 100° C. (e.g., 80° C., 85° C., 90° C., 95° C., etc.), the organosilica material may have one or more of the following:
(i) a total surface area of about 200 m²/g to about 1400 m²/g, particularly about 400 m²/g to about 1300 m²/g, and particularly about 400 m²/g to about 1200 m²/g;
(ii) a microporous surface area of about 200 m²/g to about 600 m²/g, particularly about 200 m²/g to about 500 m²/g;
(iii) a pore volume of about 0.2 cm³/g to about 1.0 cm³/g, particularly about 0.2 cm³/g to about 0.8 cm³/g; and
(iv) an average pore radius of about 0.5 nm to about 2.0 nm, particularly about 0.5 nm to about 2.0 nm, and particularly about 1.0 nm to about 1.5 nm.

Additionally or alternatively, when the solution is aged for greater than about 7 hours to about 150 hours (e.g., 23, 48, 72, 144 hours) at a temperature of about 80° C. to about 100° C. (e.g., 80° C., 85° C., 90° C., 95° C., etc.), the organosilica material may have one or more of the following:
(i) a total surface area of about 600 m²/g to about 1400 m²/g, particularly about 800 m²/g to about 1400 m²/g, and particularly about 800 m²/g to about 1200 m²/g;
(ii) substantially no microporous surface area;
(iii) a pore volume of about 0.8 cm³/g to about 1.4 cm³/g, particularly about 0.9 cm³/g to about 1.4 cm³/g; and
(iv) an average pore radius of about 1.0 nm to about 4.0 nm, particularly about 1.0 nm to about 4.0 nm.

Additionally or alternatively, when the solution is aged for about 1 hour to about 7 hours (e.g., 1, 2, 3, 4, 5, 6 hours) at a temperature of about 110° C. to about 130° C. (e.g., 110° C., 115° C., 120° C., 125° C., etc.), the organosilica material may have one or more of the following:
(i) a pore volume of about 1.0 cm³/g to about 1.8 cm³/g, particularly about 1.2 cm³/g to about 1.8 cm³/g, particularly about 1.4 cm³/g to about 1.7 cm³/g; and
(ii) an average pore diameter of about 2.0 nm to about 8.0 nm, particularly 4.0 nm to about 6.0 nm.

Additionally or alternatively, when the solution is aged for greater than about 7 hours to about 150 hours (e.g., 23, 48, 72, 144 hours) at a temperature of about 110° C. to about 130° C. (e.g., 110° C., 115° C., 120° C., 125° C., etc.), the organosilica material may have one or more of the following:
a pore volume of about 1.0 cm³/g to about 1.8 cm³/g, particularly about 1.2 cm³/g to about 1.8 cm³/g; and
(ii) an average pore diameter of about 8.0 nm to about 16.0 nm, particularly about 10.0 nm to about 16.0 nm, particularly about 10.0 nm to about 14.0 nm.

Thus, at shorter aging times (e.g., 7, 6, 5, 4 hours, etc.) the surface area of an organosilica material made is microporous and mesoporous, but as aging time increase, the surface area transitions to primarily mesoporous. Further, as aging time increases, pore volume, average pore radius and average pore diameter increases. Increasing aging temperature along with aging time, accelerates the above-described surface area transition and increase in pore volume, average pore radius and average pore diameter.

II.I. Drying the Pre-Product

The methods described herein comprise drying the pre-product (e.g., a gel) to produce an organosilica material.

In some embodiments, the pre-product (e.g., a gel) formed in the method can be dried at a temperature of greater than or equal to about 50° C., greater than or equal to about 70° C., greater than or equal to about 80° C., greater than or equal to about 100° C., greater than or equal to about 110° C., greater than or equal to about 120° C., greater than or equal to about 150° C., greater than or equal to about 200° C., greater than or equal to about 250° C., greater than or equal to about 300° C., greater than or equal to about 350° C., greater than or equal to about 400° C., greater than or equal to about 450° C., greater than or equal to about 500° C., greater than or equal to about 550° C., or greater than or equal to about 600° C.

Additionally or alternatively, the pre-product (e.g., a gel) formed in the method can be dried at temperature of about 50° C. to about 600° C., about 50° C. to about 550° C., about 50° C. to about 500° C., about 50° C. to about 450° C., about 50° C. to about 400° C., about 50° C. to about 350° C., about 50° C. to about 300° C., about 50° C. to about 250° C., about 50° C. to about 200° C., about 50° C. to about 150° C., about 50° C. to about 120° C., about 50° C. to about 110° C., about 50° C. to about 100° C., about 50° C. to about 80° C., about 50° C. to about 70° C., about 70° C. to about 600° C., about 70° C. to about 550° C., about 70° C. to about 500° C., about 70° C. to about 450° C., about 70° C. to about 400° C., about 70° C. to about 350° C., about 70° C. to about 300° C., about 70° C. to about 250° C., about 70° C. to about 200° C., about 70° C. to about 150° C., about 70° C. to about 120° C., about 70° C. to about 110° C., about 70° C. to about 100° C., about 70° C. to about 80° C., about 80° C. to about 600° C., about 70° C. to about 550° C., about 80° C. to about 500° C., about 80° C. to about 450° C., about 80° C. to about 400° C., about 80° C. to about 350° C., about 80° C. to about 300° C., about 80° C. to about 250° C., about 80° C. to about 200° C., about 80° C. to about 150° C., about 80° C. to about 120° C., about 80° C. to about 110° C., or about 80° C. to about 100° C.

In a particular embodiment, the pre-product (e.g., a gel) formed in the method can be dried at temperature from about 70° C. to about 200° C.

Additionally or alternatively, the pre-product (e.g., a gel) formed in the method can be dried in a N₂ and/or air atmosphere.

II.K. Optional Further Steps

In some embodiments, the method can further comprise calcining the organosilica material to obtain a silica material. The calcining can be performed in air or an inert gas, such as nitrogen or air enriched in nitrogen. Calcining can take place at a temperature of at least about 300° C., at least about 350° C., at least about 400° C., at least about 450° C., at least about 500° C., at least about 550° C., at least about 600° C., or at least about 650° C., for example at least about 400° C. Additionally or alternatively, calcining can be performed at a temperature of about 300° C. to about 650° C., about 300° C. to about 600° C., about 300° C. to about 550° C., about 300° C. to about 400° C., about 300° C. to about 450° C., about 300° C. to about 400° C., about 300° C. to about 350° C., about 350° C. to about 650° C., about 350° C. to about 600° C., about 350° C. to about 550° C., about 350° C. to about 400° C., about 350° C. to about 450° C., about 350° C. to about 400° C., about 400° C. to about 650° C., about 400° C. to about 600° C., about 400° C. to about 550° C., about 400° C. to about 500° C., about 400° C. to about 450° C., about 450° C. to about 650° C., about 450° C. to about 600° C., about 450° C. to about 550° C., about 450° C. to about 500° C., about 500° C. to about 650° C., about 500° C. to about 600° C., about 500° C. to about 550° C., about 550° C. to about 650° C., about 550° C. to about 600° C. or about 600° C. to about 650° C.

In some embodiments, the method can further comprise incorporating a catalyst metal within the pores of the organosilica material. Exemplary catalyst metals can include, but are not limited to, a Group 6 element, a Group 8 element, a Group 9 element, a Group 10 element or a combination thereof. Exemplary Group 6 elements can include, but are not limited to, chromium, molybdenum, and/or tungsten, particularly including molybdenum and/or tungsten. Exemplary Group 8 elements can include, but are not limited to, iron, ruthenium, and/or osmium. Exemplary Group 9 elements can include, but are not limited to, cobalt, rhodium, and/or iridium, particularly including cobalt. Exemplary Group 10 elements can include, but are not limited to, nickel, palladium and/or platinum.

The catalyst metal can be incorporated into the organosilica material by any convenient method, such as by impregnation, by ion exchange, or by complexation to surface sites. The catalyst metal so incorporated may be employed to promote any one of a number of catalytic transformations commonly conducted in petroleum refining or petrochemicals production. Examples of such catalytic processes can include, but are not limited to, hydrogenation, dehydrogenation, aromatization, aromatic saturation, hydrodesulfurization, olefin oligomerization, polymerization, hydrodenitrogenation, hydrocracking, naphtha reforming, paraffin isomerization, aromatic transalkylation, saturation of double/triple bonds, and the like, as well as combinations thereof.

Thus, in another embodiment, a catalyst material comprising the organosilica material described herein is provided. The catalyst material may optionally comprise a binder or be self-bound. Suitable binders, include but are not limited to active and inactive materials, synthetic or naturally occurring zeolites, as well as inorganic materials such as clays and/or oxides such as silica, alumina, zirconia, titania, silica-alumina, cerium oxide, magnesium oxide, or combinations thereof. In particular, the binder may be silica-alumina, alumina and/or a zeolite, particularly alumina. Silica-alumina may be either naturally occurring or in the form of gelatinous precipitates or gels including mixtures of silica and metal oxides. It should be noted it is recognized herein that the use of a material in conjunction with a zeolite binder material, i.e., combined therewith or present during its synthesis, which itself is catalytically active may change the conversion and/or selectivity of the finished catalyst. It is also recognized herein that inactive materials can suitably serve as diluents to control the amount of conversion if the present invention is employed in alkylation processes so that alkylation products can be obtained economically and orderly without employing other means for controlling the rate of reaction. These inactive materials may be incorporated into naturally occurring clays, e.g., bentonite and kaolin, to improve the crush strength of the catalyst under commercial operating conditions and function as binders or matrices for the catalyst. The catalysts described herein typically can comprise, in a composited form, a ratio of support material to binder material of about 100 parts support material to about zero parts binder material; about 99 parts support material to about 1 parts binder material; about 95 parts support material to about 5 parts binder material. Additionally or alternatively, the catalysts described herein typically can comprise, in a composited form, a ratio of support material to binder material ranging from about 90 parts support material to about 10 parts binder material to about 10 parts support material to about 90 parts binder material; about 85 parts support material to about 15 parts binder material to about 15 parts support material to about 85 parts binder material; about 80 parts support material to 20 parts binder material to 20 parts support material to 80 parts binder material, all ratios being by weight, typically from 80:20 to 50:50 support material:binder material, preferably from 65:35 to 35:65. Compositing may be done by conventional means including mulling the materials together followed by extrusion of pelletizing into the desired finished catalyst particles.

In some embodiments, the method can further comprise incorporating cationic metal sites into the network structure by any convenient method, such as impregnation or complexation to the surface, through an organic precursor, or by some other method. This organometallic material may be employed in a number of hydrocarbon separations conducted in petroleum refining or petrochemicals production. Examples of such compounds to be desirably separated from petrochemicals/fuels can include olefins, paraffins, aromatics, and the like.

Additionally or alternatively, the method can further comprise incorporating a surface metal within the pores of the organosilica material. The surface metal can be selected from a Group 1 element, a Group 2 element, a Group 13 element, and a combination thereof. When a Group 1 element is present, it can preferably comprise or be sodium and/or potassium. When a Group 2 element is present, it can include, but may not be limited to, magnesium and/or calcium. When a Group 13 element is present, it can include, but may not be limited to, boron and/or aluminum.

One or more of the Group 1, 2, 6, 8-10 and/or 13 elements may be present on an exterior and/or interior surface of the organosilica material. For example, one or more of the Group 1, 2 and/or 13 elements may be present in a first layer on the organosilica material and one or more of the Group 6, 8, 9 and/or 10 elements may be present in a second layer, e.g., at least partially atop the Group 1, 2 and/or 13 elements. Additionally or alternatively, only one or more Group 6, 8, 9 and/or 10 elements may present on an exterior and/or interior surface of the organosilica material. The surface metal(s) can be incorporated into/onto the organosilica material by any convenient method, such as by impregnation, deposition, grafting, co-condensation, by ion exchange, and/or the like.

### III. ORGANOSILICA MATERIAL

Organosilica materials can be made by the methods described herein.

The organosilica materials made by the methods described herein can be polymers comprising independent siloxane units of Formula [Z³Z⁴SiCH₂]₃ (I), wherein each Z³ represents a hydroxyl group, a C₁-C₄ alkoxy group or an oxygen atom bonded to a silicon atom of another siloxane unit and each Z⁴ represents a hydroxyl group, a C₁-C₄ alkoxy group, a C₁-C₄ alkyl group, or an oxygen atom bonded to a silicon atom of another siloxane.

In one embodiment, each Z³ can be a hydroxyl group.

Additionally or alternatively, each Z³ can be a C₁-C₄ alkoxy group, a C₁-C₃ alkoxy group, a C₁-C₂ alkoxy group, or methoxy.

Additionally or alternatively, each Z³ can be an oxygen atom bonded to a silicon atom of another siloxane unit.

Additionally or alternatively, each Z³ can be a hydroxyl group, a C₁-C₂ alkoxy group, or an oxygen atom bonded to a silicon atom of another siloxane unit.

Additionally or alternatively, each Z⁴ can be a hydroxyl group.

Additionally or alternatively, each Z⁴ can be a C₁-C₄ alkoxy group, a C₁-C₃ alkoxy group, a C₁-C₂ alkoxy group, or methoxy.

Additionally or alternatively, each Z⁴ can be a C₁-C₄ alkyl group, a C₁-C₃ alkyl group, a C₁-C₂ alkyl group, or methyl.

Additionally or alternatively, each Z⁴ can be an oxygen atom bonded to a silicon atom of another siloxane unit.

Additionally or alternatively, each Z⁴ can be a hydroxyl group, a C₁-C₂ alkoxy group, a C₁-C₂ alkyl group, or an oxygen atom bonded to a silicon atom of another siloxane unit.

Additionally or alternatively, each Z³ can be a hydroxyl group, a C₁-C₂ alkoxy group, or an oxygen atom bonded to a silicon atom of another siloxane unit and each Z⁴ can be a hydroxyl group, a C₁-C₂ alkyl group, a C₁-C₂ alkoxy group, or an oxygen atom bonded to a silicon atom of another siloxane unit.

Additionally or alternatively, each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane.

Additionally or alternatively, each Z³ can be a hydroxyl group or an oxygen atom bonded to a silicon atom of another siloxane and each Z⁴ can be a hydroxyl group, or an oxygen atom bonded to a silicon atom of another siloxane.

If a compound of Formula (Ia) is used in the methods described herein, the organosilica material made can be a homopolymer comprising independent units of Formula I.

In a particular embodiment, if a compound of Formula (Ia), such as [(EtO)₂SiCH₂]₃, is used in the methods described herein, the organosilica material made can be a homopolymer comprising independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane.

In another particular embodiment, if two compounds of Formula (Ia), such as [(EtO)₂SiCH₂]₃ and [EtOCH₃SiCH₂]₃, are used in the methods described herein, the organosilica material made can be a copolymer comprising: independent units of Formula I, wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (I), wherein each Z³ can be a hydroxyl group, ethoxy, or an oxygen atom bonded to a silicon atom of another siloxane and each Z⁴ can be methyl.

If a compound of Formula (Ia) and a compound of Formula (II) are used in the methods described herein, the organosilica material made can be a copolymer comprising independent units of Formula I and independent units of Formula Z¹¹OZ¹²Z¹³Z¹⁴ (VI), wherein each Z¹¹ can be a hydrogen atom or a C₁-C₄ alkyl group or a bond to a silicon atom of another monomer; and Z¹², Z¹³ and Z¹⁴ each independently can be selected from the group consisting of a hydroxyl group, a C₁-C₄ alkyl group, a C₁-C₄ alkoxy group, a nitrogen-containing C₁-C₁₀ alkyl group, a nitrogen-containing heteroalkyl group, a nitrogen-containing optionally substituted heterocycloalkyl group and an oxygen atom bonded to a silicon atom of another monomer. As used herein, and unless otherwise specified, “a bond to a silicon atom of another monomer” means the bond can advantageously displace a moiety (particularly an oxygen-containing moiety such as a hydroxyl, an alkoxy or the like), if present, on a silicon atom of the another monomer so there may be a bond directly to the silicon atom of the another monomer thereby connecting the two monomers, e.g., via a Si—O—Si linkage. For clarity, in this bonding scenario, the “another monomer” can be a monomer of the same type or a monomer of a different type.

In another particular embodiment, if a compound of Formula (Ia), such as [(EtO)₂SiCH₂]₃, and compound of Formula (II), such as tetraethyl orthosilicate (TEOS), are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, ethyl or a bond to a silicon atom of another monomer; and Z¹², Z¹³ and Z¹⁴ each independently can be selected from the group consisting of a hydroxyl group, ethoxy, and an oxygen atom bonded to a silicon atom of another monomer.

In another particular embodiment, if a compound of Formula (Ia), such as [(EtO)₂SiCH₂]₃, and compound of Formula (II), such as methyltriethoxysilane (MTES), are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, ethyl or a bond to a silicon atom of another monomer; Z¹², Z¹³ each independently can be selected from the group consisting of a hydroxyl group, ethoxy, and an oxygen atom bonded to a silicon atom of another monomer; and each Z¹⁴ can be methyl.

In another particular embodiment, if a compound of Formula (Ia), such as [(EtO)₂SiCH₂]₃, and compound of Formula (II), such as (N,N-dimethylaminopropyl)-trimethoxysilane, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, methyl or a bond to a silicon atom of another monomer; Z¹², Z¹³ each independently can be selected from the group consisting of a hydroxyl group, methoxy, and an oxygen atom bonded to a silicon atom of another monomer; and Z¹⁴ can be

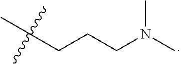

In another particular embodiment, if a compound of Formula (Ia), such as [(EtO)₂SiCH₂]₃, and compound of Formula (II), such as (N-(2-aminoethyl)-3-aminopropyl)triethoxysilane, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, ethyl or a bond to a silicon atom of another monomer; Z¹², Z¹³ each independently can be selected from the group consisting of a hydroxyl group, ethoxy, and an oxygen atom bonded to a silicon atom of another monomer; and each Z¹⁴ can be

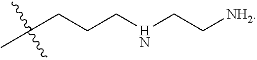

In another particular embodiment, if a compound of Formula (Ia), such as [(EtO)₂SiCH₂]₃, and compound of Formula (II), such as 4-methyl-1-(3-triethoxysilyl-propyl)-piperazine, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, ethyl or a bond to a silicon atom of another monomer; Z¹², Zⁿ each independently can be selected from the group consisting of a hydroxyl group, ethoxy, and an oxygen atom bonded to a silicon atom of another monomer; and each Z¹⁴ can be

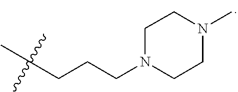

In another particular embodiment, if a compound of Formula (Ia), such as [(EtO)₂SiCH₂]₃, and compound of Formula (II), such as 4-(2-(triethoxysily)ethyl)-pyridine, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, ethyl or a bond to a silicon atom of another monomer; Z¹², Z¹³ each independently can be selected from the group consisting of a hydroxyl group, ethoxy, and an oxygen atom bonded to a silicon atom of another monomer; and each Z¹⁴ can be

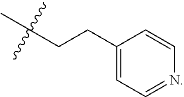

In another particular embodiment, if a compound of Formula (Ia), such as [(EtO)₂SiCH₂]₃, and compound of Formula (II), such as 1-(3-(triethoxysilyl)propyl)-4,5-dihydro-1H-imidazole, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, ethyl or a bond to a silicon atom of another monomer; Z¹², Z¹³ each independently can be selected from the group consisting of a hydroxyl group, ethoxy, and an oxygen atom bonded to a silicon atom of another monomer; and each Z¹⁴ can be

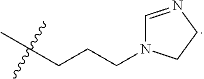

In another particular embodiment, if a compound of Formula (Ia), such as [(EtO)₂SiCH₂]₃, and compound of Formula (II), such as (3-aminopropyl)triethoxysilane, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VI), wherein each Z¹¹ can be a hydrogen atom, ethyl or a bond to a silicon atom of another monomer; Z¹², Z¹³ each independently can be selected from the group consisting of a hydroxyl group, ethoxy, and an oxygen atom bonded to a silicon atom of another monomer; and each Z¹⁴ can be

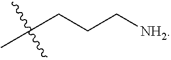

If a compound of Formula (Ia) and a compound of Formula (III) are used in the methods described herein, the organosilica material made can be a copolymer comprising independent units of Formula I and independent units of Formula Z¹⁵Z¹⁶Z¹⁷Si—R⁵—SiZ¹⁵Z¹⁶Z¹⁷ (VII), wherein each Z¹⁵ independently can be a hydroxyl group, a C₁-C₄ alkoxy group or an oxygen atom bonded to a silicon atom of another comonomer; each Z¹⁶ and Z¹⁷ independently can be a hydroxyl group, a C₁-C₄ alkoxy group, a C₁-C₄ alkyl group or an oxygen atom bonded to a silicon atom of another monomer; and each R⁵ can be selected from the group consisting of a C₁-C₈ alkylene group, a C₂-C₈ alkenylene group, a C₂-C₈ alkynylene group, a nitrogen-containing C₁-C₁₀ alkylene group, an optionally substituted C₆-C₂₀ aralkyl and an optionally substituted C₄-C₂₀ heterocycloalkyl group.

In another particular embodiment, if a compound of Formula (Ia), such as [(EtO)₂SiCH₂]₃, and compound of Formula (III), such as (1,2-bis(methyldiethoxysilyl)-ethane, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VII), wherein each Z¹⁵ can be a hydroxyl group, an ethoxy or an oxygen atom bonded to a silicon atom of another comonomer; each Z¹⁶ can be a hydroxyl group, an ethoxy group or an oxygen atom bonded to a silicon atom of another monomer; each Z¹⁷ can be methyl; and each R⁵ can be —CH₂CH₂—.

In another particular embodiment, if a compound of Formula (Ia), such as [(EtO)₂SiCH₂]₃, and compound of Formula (III), such as (bis(triethoxysilyl)methane, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen bonded to a silicon atom of another siloxane; and independent units of Formula (VII), wherein each Z¹⁵ can be a hydroxyl group, an ethoxy or an oxygen atom bonded to a silicon atom of another comonomer; each Z¹⁶ and Z¹⁷ can be independently selected from the group consisting of a hydroxyl group, an ethoxy group or an oxygen atom bonded to a silicon atom of another monomer; and each R⁵ can be —CH₂—.

In another particular embodiment, if a compound of Formula (Ia), such as [(EtO)₂SiCH₂]₃, and compound of Formula (III), such as 1,2-bis(triethoxysilyl)-ethylene, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VII), wherein each Z¹⁵ can be a hydroxyl group, an ethoxy or an oxygen atom bonded to a silicon atom of another comonomer; each Z¹⁶ and Z¹⁷ can be independently selected from the group consisting of a hydroxyl group, an ethoxy group or an oxygen atom bonded to a silicon atom of another monomer; and each R⁵ can be —HC═CH—.

In another particular embodiment, if a compound of Formula (Ia), such as [(EtO)₂SiCH₂]₃, and compound of Formula (III), such as N,N′-bis[(3-trimethoxysilyl)-propyl]ethylenediamine, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VII), wherein each Z¹⁵ can be a hydroxyl group, an methoxy or an oxygen atom bonded to a silicon atom of another comonomer; each Z¹⁶ and Z¹⁷ can be independently selected from the group consisting of a hydroxyl group, an methoxy group or an oxygen atom bonded to a silicon atom of another monomer; and each R⁵ can be

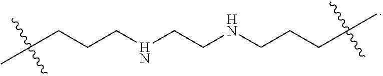 [SMILES: *CCCNCCNCCC*]

In another particular embodiment, if a compound of Formula (Ia), such as [(EtO)₂SiCH₂]₃, and compound of Formula (III), such as bis[(methyldiethoxysilyl)-propyl]amine, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VII), wherein each Z¹⁵ can be a hydroxyl group, an ethoxy or an oxygen atom bonded to a silicon atom of another comonomer; each Z¹⁶ can be a hydroxyl group, an ethoxy group or an oxygen atom bonded to a silicon atom of another monomer; each Z¹⁷ can be methyl; and each R⁵ can be

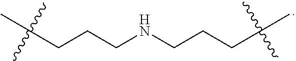

In another particular embodiment, if a compound of Formula (Ia), such as [(EtO)₂SiCH₂]₃, and compound of Formula (III), such as bis[(methyldimethoxysilyl)-propyl]-N-methylamine, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VII), wherein each Z¹⁵ can be a hydroxyl group, a methoxy or an oxygen atom bonded to a silicon atom of another comonomer; each Z¹⁶ can be a hydroxyl group, a methoxy group or an oxygen atom bonded to a silicon atom of another monomer; each Z¹⁷ can be methyl; and each R⁵ can be

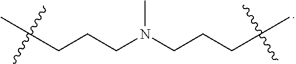

If a compound of Formula (Ia) and a compound of Formula (IV) are used in the methods described herein, the organosilica material made can be a copolymer comprising independent units of Formula I and independent units of Formula M³(OZ¹⁸)₃ (VIII), wherein M³ can be a Group 13 metal and each Z¹⁸ independently can be a hydrogen atom, a C₁-C₆ alkyl or a bond to a silicon atom of another monomer.

In another particular embodiment, if a compound of Formula (Ia), such as [(EtO)₂SiCH₂]₃, and compound of Formula (IV), such as aluminum tri-sec-butoxide, are used in the methods described herein, the organosilica material made can be a copolymer comprising: independent units of Formula (I), wherein each Z³ can be a hydroxyl group, ethoxy, or an oxygen atom bonded to a silicon atom of another siloxane and each Z⁴ can be a hydroxyl group, ethoxy, or an oxygen atom bonded to a silicon atom of another siloxane; and independent units of Formula (VIII), wherein M¹³ can be a Group 13 metal and each Z¹⁸ can be a hydrogen atom, a sec-butyl or a bond to a silicon atom of another monomer.

If a compound of Formula (Ia) and a compound of Formula (V) are used in the methods described herein, the organosilica material made can be a copolymer comprising independent units of Formula I and independent units of Formula (Z¹⁹O)₂. M⁴-O—Si(OZ²⁰)₃ (IX), wherein M⁴ represents a Group 13 metal and each Z¹⁹ and each Z²⁰ independently represent a hydrogen atom, a C₁-C₆ alkyl group or a bond to a silicon atom of another monomer.

If a compound of Formula (III) and a compound of Formula (II) are used in the methods described herein, the organosilica material made can be a copolymer comprising units of Formula Z¹⁵Z¹⁶Z¹⁷Si—R⁵—SiZ¹⁵Z¹⁶Z¹⁷ (VII), wherein each Z¹⁵ can be a hydroxyl group, a C₁-C₄ alkoxy group or an oxygen bonded to a silicon atom of another comonomer; each Z¹⁶ and Z¹⁷ independently can be a hydroxyl group, a C₁-C₄ alkoxy group, a C₁-C₄ alkyl group or an oxygen bonded to a silicon atom of another monomer; and R⁵ can be selected from the group consisting of a C₁-C₈ alkylene group, a C₂-C₈ alkenylene group, a C₂-C₈ alkynylene group, a nitrogen-containing C₁-C₁₀ alkylene group, an optionally substituted C₆-C₂₀ aralkyl and an optionally substituted C₄-C₂₀ heterocycloalkyl group; and units of Formula Z¹¹OZ¹²Z¹³Z¹⁴ (VI), wherein Z¹¹ can be a hydrogen atom or a C₁-C₄ alkyl group or a bond to a silicon atom of another monomer; and Z¹², Z¹³ and Z¹⁴ each independently can be selected from the group consisting of a hydroxyl group, a C₁-C₄ alkyl group, a C₁-C₄ alkoxy group, a nitrogen-containing C₁-C₁₀ alkyl group, a nitrogen-containing heteroalkyl group, a nitrogen-containing optionally substituted heterocycloalkyl group and an oxygen atom bonded to a silicon atom of another monomer.

For example, if a compound of Formula Z⁵Z⁶Z⁷Si—R⁵—Si Z⁵Z⁶Z⁷ (III), wherein each Z⁵ represents a C₁-C₄ alkoxy group; each Z⁶ and Z⁷ independently represent a C₁-C₄ alkoxy group or a C₁-C₄ alkyl group; and R⁵ is methylene or ethylene and a compound of Formula (II), such as a tetralkyl orthosilicate, are used in the methods described herein, the organisilica material made can be a copolymer comprising: units of Formula Z¹⁵Z¹⁶Z¹⁷Si—R⁵—SiZ¹⁵Z¹⁶Z¹⁷ (VII), wherein each Z¹⁵ can be a hydroxyl group, a C₁-C₄ alkoxy group or an oxygen bonded to a silicon atom of another comonomer; each Z¹⁶ and Z¹⁷ independently can be a hydroxyl group, a C₁-C₄ alkoxy group, a C₁-C₄ alkyl group or an oxygen bonded to a silicon atom of another monomer; and R⁵ is a methylene or ethylene group; and units of Formula Z¹¹OZ¹²Z¹³Z¹⁴ (VI), wherein Z¹¹ can be a hydrogen atom or a C₁-C₄ alkyl group or a bond to a silicon atom of another monomer; and Z¹², Z¹³ and Z¹⁴ each independently can be selected from the group consisting of a hydroxyl group, a C₁-C₄ alkoxy group and an oxygen atom bonded to a silicon atom of another monomer.

In another particular embodiment, if a compound of Formula (III), such as (bis(triethoxysilyl)methane and a compound of Formula (II) such as tetraethyl orthosilicate (TEOS), are used in the methods described herein, the organosilica material made can be a copolymer comprising: units of Formula Z¹⁵Z¹⁶Z¹⁷Si—R⁵—SiZ¹⁵Z¹⁶Z¹⁷ (VII), wherein each Z¹⁵, Z¹⁶ and Z¹⁷ independently can be a hydroxyl group, an ethoxy group or an oxygen bonded to a silicon atom of another comonomer; and R⁵ is a methylene group; and units of Formula Z¹¹OZ¹²Z¹³Z¹⁴ (VI), wherein Z¹¹ can be a hydrogen atom or an ethyl group or a bond to a silicon atom of another monomer; hydroxyl group, an ethoxy group and an oxygen atom bonded to a silicon atom of another monomer.

The organosilica materials made by the methods described herein can be characterized as described in the following sections.

III.A. X-Ray Diffraction Peaks

The organosilica materials made by the methods described herein can exhibit powder X-ray diffraction patterns with one broad peak between about 1 and about 4 degrees 2θ, particularly one broad peak between about 1 and about 3 degrees 2θ. Additionally or alternatively, the organosilica materials can exhibit substantially no peaks in the range of about 0.5 to about 10 degrees 2θ, about 0.5 to about 12 degrees 2θ range, about 0.5 to about 15 degrees 2θ, about 0.5 to about 20 degrees 2θ, about 0.5 to about 30 degrees 2θ, about 0.5 to about 40 degrees 2θ, about 0.5 to about 50 degrees 2θ, about 0.5 to about 60 degrees 2θ, about 0.5 to about 70 degrees 2θ, about 2 to about 10 degrees 2θ, about 2 to about 12 degrees 2θ range, about 2 to about 15 degrees 2θ, about 2 to about 20 degrees 2θ, about 2 to about 30 degrees 2θ, about 2 to about 40 degrees 2θ, about 2 to about 50 degrees 2θ, about 2 to about 60 degrees 2θ, about 2 to about 70 degrees 2θ, about 3 to about 10 degrees 2θ, about 3 to about 12 degrees 2θ range, about 3 to about 15 degrees 2θ, about 3 to about 20 degrees 2θ, about 3 to about 30 degrees 2θ, about 3 to about 40 degrees 2θ, about 3 to about 50 degrees 2θ, about 3 to about 60 degrees 2θ, or about 3 to about 70 degrees 2θ.

III.B Silanol Content

The organosilica materials obtainable by the method of the invention can have a silanol content that varies within wide limits, depending on the composition of the synthesis solution. The silanol content can conveniently be determined by solid state silicon NMR.

III.C. Pore Size

The organosilica material produced by the methods described herein are advantageously in a mesoporous form. As indicated previously, the term mesoporous refers to solid materials having pores with a diameter within the range of from about 2 nm to about 50 nm. The average pore diameter of the organosilica material can be determined, for example, using nitrogen adsorption-desorption isotherm techniques within the expertise of one of skill in the art, such as the BET (Brunauer Emmet Teller) method.

The organosilica material can have an average pore diameter of about 0.2 nm, about 0.4 nm, about 0.5 nm, about 0.6 nm, about 0.8 nm, about 1.0 nm, about 1.5 nm, about 1.8 nm or less than about 2.0 nm.

Additionally or alternatively, the organosilica material can advantageously have an average pore diameter within the mesopore range of about 2.0 nm, about 2.5 nm, about 3.0 nm, about 3.1 nm, about 3.2 nm, about 3.3 nm, about 3.4 nm, about 3.5 nm, about 3.6 nm, about 3.7 nm, about 3.8 nm, about 3.9 nm about 4.0 nm, about 4.1 nm, about 4.5 nm, about 5.0 nm, about 6.0 nm, about 7.0 nm, about 7.3 nm, about 8 nm, about 8.4 nm, about 9 nm, about 10 nm, about 11 nm, about 13 nm, about 15 nm, about 18 nm, about 20 nm, about 23 nm, about 25 nm, about 30 nm, about 40 nm, about 45 nm, or about 50 nm.

Additionally or alternatively, the organosilica material can have an average pore diameter of 0.2 nm to about 50 nm, about 0.2 nm to about 40 nm, about 0.2 nm to about 30 nm, about 0.2 nm to about 25 nm, about 0.2 nm to about 23 nm, about 0.2 nm to about 20 nm, about 0.2 nm to about 18 nm, about 0.2 nm to about 15 nm, about 0.2 nm to about 13 nm, about 0.2 nm to about 11 nm, about 0.2 nm to about 10 nm, about 0.2 nm to about 9 nm, about 0.2 nm to about 8.4 nm, about 0.2 nm to about 8 nm, about 0.2 nm to about 7.3 nm, about 0.2 nm to about 7.0 nm, about 0.2 nm to about 6.0 nm, about 0.2 nm to about 5.0 nm, about 0.2 nm to about 4.5 nm, about 0.2 nm to about 4.1 nm, about 0.2 nm to about 4.0 nm, about 0.2 nm to about 3.9 nm, about 0.2 nm to about 3.8 nm, about 0.2 nm to about 3.7 nm, about 0.2 nm to about 3.6 nm, about 0.2 nm to about 3.5 nm, about 0.2 nm to about 3.4 nm, about 0.2 nm to about 3.3 nm, about 0.2 nm to about 3.2 nm, about 0.2 nm to about 3.1 nm, about 0.2 nm to about 3.0 nm, about 0.2 nm to about 2.5 nm, about 0.2 nm to about 2.0 nm, about 0.2 nm to about 1.0 nm, about 1.0 nm to about 50 nm, about 1.0 nm to about 40 nm, about 1.0 nm to about 30 nm, about 1.0 nm to about 25 nm, about 1.0 nm to about 23 nm, about 1.0 nm to about 20 nm, about 1.0 nm to about 18 nm, about 1.0 nm to about 15 nm, about 1.0 nm to about 13 nm, about 1.0 nm to about 11 nm, about 1.0 nm to about 10 nm, about 1.0 nm to about 9 nm, about 1.0 nm to about 8.4 nm, about 1.0 nm to about 8 nm, about 1.0 nm to about 7.3 nm, about 1.0 nm to about 7.0 nm, about 1.0 nm to about 6.0 nm, about 1.0 nm to about 5.0 nm, about 1.0 nm to about 4.5 nm, about 1.0 nm to about 4.1 nm, about 1.0 nm to about 4.0 nm, about 1.0 nm to about 3.9 nm, about 1.0 nm to about 3.8 nm, about 1.0 nm to about 3.7 nm, about 1.0 nm to about 3.6 nm, about 1.0 nm to about 3.5 nm, about 1.0 nm to about 3.4 nm, about 1.0 nm to about 3.3 nm, about 1.0 nm to about 3.2 nm, about 1.0 nm to about 3.1 nm, about 1.0 nm to about 3.0 nm or about 1.0 nm to about 2.5 nm.

In particular, the organosilica material can advantageously have an average pore diameter in the mesopore range of about 2.0 nm to about 50 nm, about 2.0 nm to about 40 nm, about 2.0 nm to about 30 nm, about 2.0 nm to about 25 nm, about 2.0 nm to about 23 nm, about 2.0 nm to about 20 nm, about 2.0 nm to about 18 nm, about 2.0 nm to about 15 nm, about 2.0 nm to about 13 nm, about 2.0 nm to about 11 nm, about 2.0 nm to about 10 nm, about 2.0 nm to about 9 nm, about 2.0 nm to about 8.4 nm, about 2.0 nm to about 8 nm, about 2.0 nm to about 7.3 nm, about 2.0 nm to about 7.0 nm, about 2.0 nm to about 6.0 nm, about 2.0 nm to about 5.0 nm, about 2.0 nm to about 4.5 nm, about 2.0 nm to about 4.1 nm, about 2.0 nm to about 4.0 nm, about 2.0 nm to about 3.9 nm, about 2.0 nm to about 3.8 nm, about 2.0 nm to about 3.7 nm, about 2.0 nm to about 3.6 nm, about 2.0 nm to about 3.5 nm, about 2.0 nm to about 3.4 nm, about 2.0 nm to about 3.3 nm, about 2.0 nm to about 3.2 nm, about 2.0 nm to about 3.1 nm, about 2.0 nm to about 3.0 nm, about 2.0 nm to about 2.5 nm, about 2.5 nm to about 50 nm, about 2.5 nm to about 40 nm, about 2.5 nm to about 30 nm, about 2.5 nm to about 25 nm, about 2.5 nm to about 23 nm, about 2.5 nm to about 20 nm, about 2.5 nm to about 18 nm, about 2.5 nm to about 15 nm, about 2.5 nm to about 13 nm, about 2.5 nm to about 11 nm, about 2.5 nm to about 10 nm, about 2.5 nm to about 9 nm, about 2.5 nm to about 8.4 nm, about 2.5 nm to about 8 nm, about 2.5 nm to about 7.3 nm, about 2.5 nm to about 7.0 nm, about 2.5 nm to about 6.0 nm, about 2.5 nm to about 5.0 nm, about 2.5 nm to about 4.5 nm, about 2.5 nm to about 4.1 nm, about 2.5 nm to about 4.0 nm, about 2.5 nm to about 3.9 nm, about 2.5 nm to about 3.8 nm, about 2.5 nm to about 3.7 nm, about 2.5 nm to about 3.6 nm, about 2.5 nm to about 3.5 nm, about 2.5 nm to about 3.4 nm, about 2.5 nm to about 3.3 nm, about 2.5 nm to about 3.2 nm, about 2.5 nm to about 3.1 nm, about 2.5 nm to about 3.0 nm, about 3.0 nm to about 50 nm, about 3.0 nm to about 40 nm, about 3.0 nm to about 30 nm, about 3.0 nm to about 25 nm, about 3.0 nm to about 23 nm, about 3.0 nm to about 20 nm, about 3.0 nm to about 18 nm, about 3.0 nm to about 15 nm, about 3.0 nm to about 13 nm, about 3.0 nm to about 11 nm, about 3.0 nm to about 10 nm, about 3.0 nm to about 9 nm, about 3.0 nm to about 8.4 nm, about 3.0 nm to about 8 nm, about 3.0 nm to about 7.3 nm, about 3.0 nm to about 7.0 nm, about 3.0 nm to about 6.0 nm, about 3.0 nm to about 5.0 nm, about 3.0 nm to about 4.5 nm, about 3.0 nm to about 4.1 nm, or about 3.0 nm to about 4.0 nm.

In one particular embodiment, the organosilica material produced by the methods described herein can have an average pore diameter of about 1.0 nm to about 30.0 nm, particularly about 1.0 nm to about 25.0 nm, particularly about 1.5 nm to about 25.0 nm, particularly about 2.0 nm to about 25.0 nm, particularly about 2.0 nm to about 20.0 nm, particularly about 2.0 nm to about 15.0 nm, or particularly about 2.0 nm to about 10.0 nm.

Using surfactant as a template to synthesize mesoporous materials can create highly ordered structure, e.g. well-defined cylindrical-like pore channels. In some circumstances, there may be no hysteresis loop observed from N₂ adsorption isotherm. In other circumstances, for instance where mesoporous materials can have less ordered pore structures, a hysteresis loop may be observed from N₂ adsorption isotherm experiments. In such circumstances, without being bound by theory, the hysteresis can result from the lack of regularity in the pore shapes/sizes and/or from bottleneck constrictions in such irregular pores.

III.D. Surface Area

The surface area of the organosilica material can be determined, for example, using nitrogen adsorption-desorption isotherm techniques within the expertise of one of skill in the art, such as the BET (Brunauer Emmet Teller) method. This method may determine a total surface area, an external surface area, and a microporous surface area. As used herein, and unless otherwise specified, “total surface area” refers to the total surface area as determined by the BET method. As used herein, and unless otherwise specified, “microporous surface area” refers to microporous surface are as determined by the BET method.

In various embodiments, the organosilica material can have a total surface area greater than or equal to about 100 m²/g, greater than or equal to about 200 m²/g, greater than or equal to about 300 m²/g, greater than or equal to about 400 m²/g, greater than or equal to about 450 m²/g, greater than or equal to about 500 m²/g, greater than or equal to about 550 m²/g, greater than or equal to about 600 m²/g, greater than or equal to about 700 m²/g, greater than or equal to about 800 m²/g, greater than or equal to about 850 m²/g, greater than or equal to about 900 m²/g, greater than or equal to about 1,000 m²/g, greater than or equal to about 1,050 m²/g, greater than or equal to about 1,100 m²/g, greater than or equal to about 1,150 m²/g, greater than or equal to about 1,200 m²/g, greater than or equal to about 1,250 m²/g, greater than or equal to about 1,300 m²/g, greater than or equal to about 1,400 m²/g, greater than or equal to about 1,450 m²/g, greater than or equal to about 1,500 m²/g, greater than or equal to about 1,550 m²/g, greater than or equal to about 1,600 m²/g, greater than or equal to about 1,700 m²/g, greater than or equal to about 1,800 m²/g, greater than or equal to about 1,900 m²/g, greater than or equal to about 2,000 m²/g, greater than or equal to greater than or equal to about 2,100 m²/g, greater than or equal to about 2,200 m²/g, greater than or equal to about 2,300 m²/g or about 2,500 m²/g.

Additionally or alternatively, the organosilica material may have a total surface area of about 50 m²/g to about 2,500 m²/g, about 50 m²/g to about 2,000 m²/g, about 50 m²/g to about 1,500 m²/g, about 50 m²/g to about 1,000 m²/g, about 100 m²/g to about 2,500 m²/g, about 100 m²/g to about 2,300 m²/g, about 100 m²/g to about 2,200 m²/g, about 100 m²/g to about 2,100 m²/g, about 100 m²/g to about 2,000 m²/g, about 100 m²/g to about 1,900 m²/g, about 100 m²/g to about 1,800 m²/g, about 100 m²/g to about 1,700 m²/g, about 100 m²/g to about 1,600 m²/g, about 100 m²/g to about 1,550 m²/g, about 100 m²/g to about 1,500 m²/g, about 100 m²/g to about 1,450 m²/g, about 100 m²/g to about 1,400 m²/g, about 100 m²/g to about 1,300 m²/g, about 100 m²/g to about 1,250 m²/g, about 100 m²/g to about 1,200 m²/g, about 100 m²/g to about 1,150 m²/g, about 100 m²/g to about 1,100 m²/g, about 100 m²/g to about 1,050 m²/g, about 100 m²/g to about 1,000 m²/g, about 100 m²/g to about 900 m²/g, about 100 m²/g to about 850 m²/g, about 100 m²/g to about 800 m²/g, about 100 m²/g to about 700 m²/g, about 100 m²/g to about 600 m²/g, about 100 m²/g to about 550 m²/g, about 100 m²/g to about 500 m²/g, about 100 m²/g to about 450 m²/g, about 100 m²/g to about 400 m²/g, about 100 m²/g to about 300 m²/g, about 100 m²/g to about 200 m²/g, about 200 m²/g to about 2,500 m²/g, about 200 m²/g to about 2,300 m²/g, about 200 m²/g to about 2,200 m²/g, about 200 m²/g to about 2,100 m²/g, about 200 m²/g to about 2,000 m²/g, about 200 m²/g to about 1,900 m²/g, about 200 m²/g to about 1,800 m²/g, about 200 m²/g to about 1,700 m²/g, about 200 m²/g to about 1,600 m²/g, about 200 m²/g to about 1,550 m²/g, about 200 m²/g to about 1,500 m²/g, about 200 m²/g to about 1,450 m²/g, about 200 m²/g to about 1,400 m²/g, about 200 m²/g to about 1, 300 m²/g, about 200 m²/g to about 1,250 m²/g, about 200 m²/g to about 1,200 m²/g, about 200 m²/g to about 1,150 m²/g, about 200 m²/g to about 1,100 m²/g, about 200 m²/g to about 1,050 m²/g, about 200 m²/g to about 1,000 m²/g, about 200 m²/g to about 900 m²/g, about 200 m²/g to about 850 m²/g, about 200 m²/g to about 800 m²/g, about 200 m²/g to about 700 m²/g, about 200 m²/g to about 600 m²/g, about 200 m²/g to about 550 m²/g, about 200 m²/g to about 500 m²/g, about 200 m²/g to about 450 m²/g, about 200 m²/g to about 400 m²/g, about 200 m²/g to about 300 m²/g, about 500 m²/g to about 2,500 m²/g, about 500 m²/g to about 2,300 m²/g, about 500 m²/g to about 2,200 m²/g, about 500 m²/g to about 2,100 m²/g, about 500 m²/g to about 2,000 m²/g, about 500 m²/g to about 1,900 m²/g, about 500 m²/g to about 1,800 m²/g, about 500 m²/g to about 1,700 m²/g, about 500 m²/g to about 1,600 m²/g, about 500 m²/g to about 1,550 m²/g, about 500 m²/g to about 1,500 m²/g, about 500 m²/g to about 1,450 m²/g, about 500 m²/g to about 1,400 m²/g, about 500 m²/g to about 1,300 m²/g, about 500 m²/g to about 1,250 m²/g, about 500 m²/g to about 1,200 m²/g, about 500 m²/g to about 1,150 m²/g, about 500 m²/g to about 1,100 m²/g, about 500 m²/g to about 1,050 m²/g, about 500 m²/g to about 1,000 m²/g, about 500 m²/g to about 900 m²/g, about 500 m²/g to about 850 m²/g, about 500 m²/g to about 800 m²/g, about 500 m²/g to about 700 m²/g, about 500 m²/g to about 600 m²/g, about 500 m²/g to about 550 m²/g, about 1,000 m²/g to about 2,500 m²/g, about 1,000 m²/g to about 2,300 m²/g, about 1,000 m²/g to about 2,200 m²/g, about 1,000 m²/g to about 2,100 m²/g, about 1,000 m²/g to about 2,000 m²/g, about 1,000 m²/g to about 1,900 m²/g, about 1,000 m²/g to about 1,800 m²/g, about 1,000 m²/g to about 1,700 m²/g, about 1,000 m²/g to about 1,600 m²/g, about 1,000 m²/g to about 1,550 m²/g, about 1,000 m²/g to about 1,500 m²/g, about 1,000 m²/g to about 1,450 m²/g, about 1,000 m²/g to about 1,400 m²/g, about 1,000 m²/g to about 1, 300 m²/g, about 1,000 m²/g to about 1,250 m²/g, about 1,000 m²/g to about 1,200 m²/g, about 1,000 m²/g to about 1,150 m²/g, about 1,000 m²/g to about 1,100 m²/g, or about 1,000 m²/g to about 1,050 m²/g.

In one particular embodiment, the organosilica material described herein may have a total surface area of about 100 m²/g to about 2,500 m²g, particularly about 200 m²/g to about 2,500 m²/g, particularly about 200 m²/g to about 2,000 m²/g, particularly about 500 m²/g to about 2,000 m²/g, or particularly about 1,000 m²/g to about 2,000 m²/g.

III.E. Pore Volume

The pore volume of the organosilica material made by the methods described herein can be determined, for example, using nitrogen adsorption-desorption isotherm techniques within the expertise of one of skill in the art, such as the BET (Brunauer Emmet Teller) method.

In various embodiments, the organosilica material can have a pore volume greater than or equal to about 0.1 cm³/g, greater than or equal to about 0.2 cm³/g, greater than or equal to about 0.3 cm³/g, greater than or equal to about 0.4 cm³/g, greater than or equal to about 0.5 cm³/g, greater than or equal to about 0.6 cm³/g, greater than or equal to about 0.7 cm³/g, greater than or equal to about 0.8 cm³/g, greater than or equal to about 0.9 cm³/g, greater than or equal to about 1.0 cm³/g, greater than or equal to about 1.1 cm³/g, greater than or equal to about 1.2 cm³/g, greater than or equal to about 1.3 cm³/g, greater than or equal to about 1.4 cm³/g, greater than or equal to about 1.5 cm³/g, greater than or equal to about 1.6 cm³/g, greater than or equal to about 1.7 cm³/g, greater than or equal to about 1.8 cm³/g, greater than or equal to about 1.9 cm³/g, greater than or equal to about 2.0 cm³/g, greater than or equal to about 2.5 cm³/g, greater than or equal to about 3.0 cm³/g, greater than or equal to about 3.5 cm³/g, greater than or equal to about 4.0 cm³/g, greater than or equal to about 5.0 cm³/g, greater than or equal to about 6.0 cm³/g, greater than or equal to about 7.0 cm³/g, or about 10.0 cm³/g.

Additionally or alternatively, the organosilica material can have a pore volume of about 0.1 cm³/g to about 10.0 cm³/g, about 0.1 cm³/g to about 7.0 cm³/g, about 0.1 cm³/g to about 6.0 cm³/g, about 0.1 cm³/g to about 5.0 cm³/g, about 0.1 cm³/g to about 4.0 cm³/g, about 0.1 cm³/g to about 3.5 cm³/g, about 0.1 cm³/g to about 3.0 cm³/g, about 0.1 cm³/g to about 2.5 cm³/g, about 0.1 cm³/g to about 2.0 cm³/g, about 0.1 cm³/g to about 1.9 cm³/g, about 0.1 cm³/g to about 1.8 cm³/g, about 0.1 cm³/g to about 1.7 cm³/g, about 0.1 cm³/g to about 1.6 cm³/g, about 0.1 cm³/g to about 1.5 cm³/g, about 0.1 cm³/g to about 1.4 cm³/g, about 0.1 cm³/g to about 1.3 cm³/g, about 0.1 cm³/g to about 1.2 cm³/g, about 0.1 cm³/g to about 1.1, about 0.1 cm³/g to about 1.0 cm³/g, about 0.1 cm³/g to about 0.9 cm³/g, about 0.1 cm³/g to about 0.8 cm³/g, about 0.1 cm³/g to about 0.7 cm³/g, about 0.1 cm³/g to about 0.6 cm³/g, about 0.1 cm³/g to about 0.5 cm³/g, about 0.1 cm³/g to about 0.4 cm³/g, about 0.1 cm³/g to about 0.3 cm³/g, about 0.1 cm³/g to about 0.2 cm³/g, 0.2 cm³/g to about 10.0 cm³/g, about 0.2 cm³/g to about 7.0 cm³/g, about 0.2 cm³/g to about 6.0 cm³/g, about 0.2 cm³/g to about 5.0 cm³/g, about 0.2 cm³/g to about 4.0 cm³/g, about 0.2 cm³/g to about 3.5 cm³/g, about 0.2 cm³/g to about 3.0 cm³/g, about 0.2 cm³/g to about 2.5 cm³/g, about 0.2 cm³/g to about 2.0 cm³/g, about 0.2 cm³/g to about 1.9 cm³/g, about 0.2 cm³/g to about 1.8 cm³/g, about 0.2 cm³/g to about 1.7 cm³/g, about 0.2 cm³/g to about 1.6 cm³/g, about 0.2 cm³/g to about 1.5 cm³/g, about 0.2 cm³/g to about 1.4 cm³/g, about 0.2 cm³/g to about 1.3 cm³/g, about 0.2 cm³/g to about 1.2 cm³/g, about 0.2 cm³/g to about 1.1, about 0.5 cm³/g to about 1.0 cm³/g, about 0.5 cm³/g to about 0.9 cm³/g, about 0.5 cm³/g to about 0.8 cm³/g, about 0.5 cm³/g to about 0.7 cm³/g, about 0.5 cm³/g to about 0.6 cm³/g, about 0.5 cm³/g to about 0.5 cm³/g, about 0.5 cm³/g to about 0.4 cm³/g, about 0.5 cm³/g to about 0.3 cm³/g, 0.5 cm³/g to about 10.0 cm³/g, about 0.5 cm³/g to about 7.0 cm³/g, about 0.5 cm³/g to about 6.0 cm³/g, about 0.5 cm³/g to about 5.0 cm³/g, about 0.5 cm³/g to about 4.0 cm³/g, about 0.5 cm³/g to about 3.5 cm³/g, about 0.5 cm³/g to about 3.0 cm³/g, about 0.5 cm³/g to about 2.5 cm³/g, about 0.5 cm³/g to about 2.0 cm³/g, about 0.5 cm³/g to about 1.9 cm³/g, about 0.5 cm³/g to about 1.8 cm³/g, about 0.5 cm³/g to about 1.7 cm³/g, about 0.5 cm³/g to about 1.6 cm³/g, about 0.5 cm³/g to about 1.5 cm³/g, about 0.5 cm³/g to about 1.4 cm³/g, about 0.5 cm³/g to about 1.3 cm³/g, about 0.5 cm³/g to about 1.2 cm³/g, about 0.5 cm³/g to about 1.1, about 0.5 cm³/g to about 1.0 cm³/g, about 0.5 cm³/g to about 0.9 cm³/g, about 0.5 cm³/g to about 0.8 cm³/g, about 0.5 cm³/g to about 0.7 cm³/g, or about 0.5 cm³/g to about 0.6 cm³/g.

### IV. USES OF THE ORGANOSILICA MATERIALS

The organosilica materials obtainable by the method of the present invention find uses in several areas.

In certain embodiments, the organosilica material described herein can be used as adsorbents or support matrices for separation and/or catalysis processes.

IV.A. Gas Separation Processes

In some cases, the organosilica materials can be used in a gas separation process as provided herein. The gas separation process can comprise contacting a gas mixture containing at least one contaminant with the organosilica material described herein as prepared according to the methods described herein.

In various embodiments, the gas separation process can be achieved by swing adsorption processes, such as pressure swing adsorption (PSA) and temperature swing adsorption (TSA). All swing adsorption processes typically have an adsorption step in which a feed mixture (typically in the gas phase) is flowed over an adsorbent to preferentially adsorb a more readily adsorbed component relative to a less readily adsorbed component. A component may be more readily adsorbed because of kinetic or equilibrium properties of the adsorbent. The adsorbent can typically be contained in a contactor that is part of the swing adsorption unit. The contactor can typically contain an engineered structured adsorbent bed or a particulate adsorbent bed. The bed can contain the adsorbent and other materials such as other adsorbents, mesopore filling materials, and/or inert materials used to mitigated temperature excursions from the heat of adsorption and desorption. Other components in the swing adsorption unit can include, but are not necessarily limited to, valves, piping, tanks, and other contactors. Swing adsorption processes are described in detail in U.S. Pat. Nos. 8,784,533; 8,784,534; 8,858,683; and 8,784,535, each of which are incorporated herein by reference. Examples of processes that can be used herein either separately or in combination are PSA, TSA, pressure temperature swing adsorption (PTSA), partial purge displacement swing adsorption (PPSA), PPTSA, rapid cycle PSA (RCPSA), RCTSA, RCPPSA and RCPTSA.

Swing adsorption processes can be applied to remove a variety of target gases, also referred to as “contaminant gas” from a wide variety of gas mixtures. Typically, in binary separation systems, the “light component” as utilized herein is taken to be the species or molecular component(s) not preferentially taken up by the adsorbent in the adsorption step of the process. Conversely in such binary systems, the “heavy component” as utilized herein is typically taken to be the species or molecular component(s) preferentially taken up by the adsorbent in the adsorption step of the process. However, in binary separation systems where the component(s) that is(are) preferentially adsorbed has(have) a lower molecular weight than the component(s) that is(are) not preferentially adsorbed, those descriptions may not necessarily correlate as disclosed above.

An example of gas mixture that can be separated in the methods described herein is a gas mixture comprising CH₄, such as a natural gas stream. A gas mixture comprising CH₄ can contain significant levels of contaminants such as H₂O, H₂S, CO₂, N₂, mercaptans, and/or heavy hydrocarbons. Additionally or alternatively, the gas mixture can comprise NOₓ and/or SOₓ species as contaminants, such as a waste gas stream, a flue gas stream and a wet gas stream. As used herein, the terms “NOₓ” and “NOₓ” species refers to the various oxides of nitrogen that may be present in waste gas, such as waste gas from combustion processes. The terms refer to all of the various oxides of nitrogen including, but not limited to, nitric oxide (NO), nitrogen dioxide (NO₂), nitrogen peroxide (N₂O), nitrogen pentoxide (N₂O₅), and mixtures thereof. As used herein, the terms “SOₓ,” and “SOₓ species,” refers to the various oxides of sulfur that may be present in waste gas, such as waste gas from combustion processes. The terms refer to all of the various oxides of sulfur including, but not limited to, SO, SO₂, SO₃, SO₄, S₇O₂ and S₆O₂. Thus, examples of contaminants include, but are not limited to H₂O, H₂S, CO₂, N₂, mercaptans, heavy hydrocarbons, NOₓ and/or SOₓ species.

IV.B. Aromatic Hydrogenation Process

The organosilica materials made according to the methods described herein can be used as support materials in hydrogenation catalysts. In particular, the hydrogenation catalyst can comprise the oraganosilica materials as a support material where the organosilica material has at least one catalyst metal incorporated on the pore surface. The at least one catalyst metal may be a Group 8 metal, a Group 9 metal, a Group 10 metal, e.g., Pt, Pd, Ir, Rh, Ru or a combination thereof. The hydrogenation catalyst can further comprise a binder such as, but not limited to, active and inactive materials, inorganic materials, clays, ceramics, activated carbon, alumina, silica, silica-alumina, titania, zirconia, niobium oxide, tantalum oxide, or a combination thereof, particularly, silica-alumina, alumina, titania, or zirconia. These hydrogenation catalysts can be used for both hydrogenation and aromatic saturation of a feedstream.

In various embodiments, the hydrogenation process can be achieved by contacting a hydrocarbon feedstream comprising aromatics with a hydrogenation catalyst described herein in the presence of a hydrogen-containing treat gas in a first reaction stage operated under effective aromatics hydrogenation conditions to produce a reaction product with reduced aromatics content.

Hydrogen-containing treat gasses suitable for use in a hydrogenation process can be comprised of substantially pure hydrogen or can be mixtures of other components typically found in refinery hydrogen streams. It is preferred that the hydrogen-containing treat gas stream contains little, more preferably no, hydrogen sulfide. The hydrogen-containing treat gas purity should be at least about 50% by volume hydrogen, preferably at least about 75% by volume hydrogen, and more preferably at least about 90% by volume hydrogen for best results. It is most preferred that the hydrogen-containing stream be substantially pure hydrogen

Feedstreams suitable for hydrogenation by the hydrogenation catalyst described herein include any conventional hydrocarbon feedstreams where hydrogenation or aromatic saturation is desirable. Such feedstreams can include hydrocarbon fluids, diesel, kerosene, lubricating oil feedstreams, heavy coker gasoil (HKGO), de-asphalted oil (DAO), FCC main column bottom (MCB), and steam cracker tar. Such feedstreams can also include other distillate feedstreams, including wax-containing feedstreams such as feeds derived from crude oils, shale oils and tar sands. Synthetic feeds such as those derived from the Fischer-Tropsch process can also be aromatically saturated using the hydrogenation catalyst described herein. Typical wax-containing feedstocks for the preparation of lubricating base oils have initial boiling points of about 315 C or higher, and include feeds such as reduced crudes, hydrocrackates, raffinates, hydrotreated oils, atmospheric gas oils, vacuum gas oils, coker gas oils, atmospheric and vacuum residues, deasphalted oils, slack waxes and Fischer-Tropsch wax. Such feeds may be derived from distillation towers (atmospheric and vacuum), hydrocrackers, hydrotreaters and solvent extraction units, and may have wax contents of up to 50% or more. Preferred lubricating oil boiling range feedstreams include feedstreams which boil in the range of 570-760° F. Diesel boiling range feedstreams include feedstreams which boil in the range of 480-660° F. Kerosene boiling range feedstreams include feedstreams which boil in the range of 350-617° F.

Hydrocarbon feedstreams suitable for use herein also contain aromatics and nitrogen- and sulfur-contaminants. Feedstreams containing up to 0.2 wt. % of nitrogen, based on the feedstream, up to 3.0 wt. % of sulfur, and up to 50 wt. % aromatics can be used in the present process In various embodiments, the sulfur content of the feedstreams can be below about 500 wppm, or below about 300 wppm, or below about 200 wppm, or below about 100 wppm, or below about 20 wppm. The pressure used during an aromatic hydrogenation process can be modified based on the expected sulfur content in a feedstream. Feeds having a high wax content typically have high viscosity indexes of up to 200 or more. Sulfur and nitrogen contents may be measured by standard ASTM methods D5453 and D4629, respectively.

Effective hydrogenation conditions may be considered to be those conditions under which at least a portion of the aromatics present in the hydrocarbon feedstream are saturated, preferably at least about 50 wt. % of the aromatics are saturated, more preferably greater than about 75 wt. %. Effective hydrogenation conditions can include temperatures of from 150° C. to 400° C., a hydrogen partial pressure of from 740 to 20786 kPa (100 to 3000 psig), a space velocity of from 0.1 to 10 liquid hourly space velocity (LHSV), and a hydrogen to feed ratio of from 89 to 1780 m³/m³ (500 to 10000 scf/B).

Additionally or alternatively, effective hydrogenation conditions may be conditions effective at removing at least a portion of the nitrogen and organically bound sulfur contaminants and hydrogenating at least a portion of said aromatics, thus producing at least a liquid diesel boiling range product having a lower concentration of aromatics and nitrogen and organically bound sulfur contaminants than the diesel boiling range feedstream.

### V. FURTHER EMBODIMENTS

The invention can additionally or alternately include one or more of the following embodiments

### Embodiment 1

A method for preparing an organosilica material is provided herein, the method comprising:
(a) providing an aqueous mixture that contains essentially no structure directing agent and/or porogen,
(b) adding at least one compound of Formula [Z¹Z²SiCH₂]₃ (Ia) into the aqueous mixture to form a solution, wherein each Z¹ represents a C₁-C₄ alkoxy group and each Z² represents a C₁-C₄ alkoxy group or a C₁-C₄ alkyl group;
(c) aging the solution to produce a pre-product; and
(d) drying the pre-product to obtain an organosilica material which is a polymer comprising independent siloxane units of Formula [Z³Z⁴SiCH₂]₃ (I), wherein each Z³ represents a hydroxyl group, a C₁-C₄ alkoxy group or an oxygen atom bonded to a silicon atom of another siloxane unit and each Z⁴ represents a hydroxyl group, a C₁-C₄ alkoxy group, a C₁-C₄ alkyl group, or an oxygen atom bonded to a silicon atom of another siloxane.

### Embodiment 2

The method of embodiment 1, wherein each Z¹ represents a C₁-C₂ alkoxy group.

### Embodiment 3

The method of embodiment 1 or 2, wherein each Z² represents a C₁-C₄ alkoxy group.

### Embodiment 4

The method of any one of the previous embodiments, wherein each Z² represents a C₁-C₂ alkoxy group.

### Embodiment 5

The method of any one of the previous embodiments, wherein the at least one compound of Formula (Ia) is 1,1,3,3,5,5-hexaethoxy-1,3,5-trisilacyclohexane.

### Embodiment 6

The method of any one of the previous embodiments, wherein each Z³ represents a hydroxyl group, a C₁-C₂ alkoxy group, or an oxygen atom bonded to a silicon atom of another siloxane unit and each Z⁴ represent a hydroxyl group, a C₁-C₂ alkyl group, a C₁-C₂ alkoxy group, or an oxygen atom bonded to a silicon atom of another siloxane unit.

### Embodiment 7

The method of any one of the previous embodiments, wherein each Z³ represents a hydroxyl group, ethoxy, or an oxygen atom bonded to a silicon atom of another siloxane and each Z⁴ represent a hydroxyl group, ethoxy, or an oxygen atom bonded to a silicon atom of another siloxane.

### Embodiment 8

The method of any one of the previous embodiments, further comprising adding to the aqueous mixture at least one compound selected from the group consisting of
(i) a further compound of Formula (Ia)
(ii) a compound of Formula R¹OR²R³R⁴Si (II), wherein each R¹ represents a C₁-C₆ alkyl group, and R², R³ and R⁴ are each independently selected from the group consisting of a C₁-C₆ alkyl group, a C₁-C₆ alkoxy group, a nitrogen-containing C₁-C₁₀ alkyl group, a nitrogen-containing heteroaralkyl group, and a nitrogen-containing optionally substituted heterocycloalkyl group;
(iii) compound of Formula Z⁵Z⁶Z⁷Si—R—SiZ⁵Z⁶Z⁷ (III), wherein each Z⁵ independently represents a C₁-C₄ alkoxy group; each Z⁶ and Z⁷ independently represent a C₁-C₄ alkoxy group or a C₁-C₄ alkyl group; and each R is selected from the group consisting a C₁-C₈ alkylene group, a C₂-C₈ alkenylene group, a C₂-C₈ alkynylene group, a nitrogen-containing C₁-C₁₀ alkylene group, an optionally substituted C₆-C₂₀ aralkyl and an optionally substituted C₄-C₂₀ heterocycloalkyl group;
(iv) a source of a trivalent metal oxide; and
(v) a combination thereof.

### Embodiment 9

The method of embodiment 8, wherein the at least one compound is a further compound of Formula (Ia), wherein each Z¹ represents a C₁-C₂ alkoxy group and each Z² represent C₁-C₂ alkoxy group or a C₁-C₂ alkyl group.

### Embodiment 10

The method of embodiment 9, wherein the compound of Formula (Ia) is 1,3,5-trimethyl-1,3,5-triethoxy-1,3,5-trisilacyclohexane.

### Embodiment 11

The method of any one of embodiments 8-10, wherein the at least one compound is a compound of Formula (II), wherein each R¹ represents a C₁-C₂ alkyl group and R², R³ and R⁴ are each independently a C₁-C₂ alkyl group, C₁-C₂ alkoxy group, a nitrogen-containing C₃-C₁₀ alkyl group, a nitrogen-containing C₄-C₁₀ heteroaralkyl group, or a nitrogen-containing optionally substituted C₄-C₁₀ heterocycloalkyl group.

### Embodiment 12

The method of embodiment 11, wherein the compound of Formula (II) is selected from the group consisting of tetraethyl orthosilicate, methyltriethoxysilane, (N,N-dimethylaminopropyl)trimethoxysilane, N-(2-aminoethyl)-3-aminopropyltriethoxysilane, 4-methyl-1-(3-triethoxysilylpropyl)-piperazine, 4-(2-(triethoxysily)ethyl)pyridine, 1-(3-(triethoxysilyl)propyl)-4,5-dihydro-1H-imidazole, and (3-aminopropyl)triethoxysilane.

### Embodiment 13

The method of any one of embodiments 8-12, wherein the at least one compound is a compound of Formula (III), wherein each Z⁵ independently represents a C₁-C₂ alkoxy group; each Z⁶ and Z⁷ independently represent a C₁-C₂ alkoxy group, or a C₁-C₂ alkyl group; and each R is selected from the group consisting of a C₁-C₄ alkylene group, a C₂-C₄ alkenylene group, a C₂-C₄ alkynylene group, and a nitrogen-containing C₄-C₁₀ alkylene group.

### Embodiment 14

The method of embodiment 13, wherein the compound of Formula (III) is selected from the group consisting of 1,2-bis(methyldiethoxysilyl)-ethane, bis(triethoxysilyl)methane, 1,2-bis(triethoxysilyl)ethylene, N,N′-bis[(3-trimethoxysilyl)propyl]ethylenediamine, bis[(methyldiethoxysilyl)propyl]amine, and bis[(methyldimethoxysilyl)propyl]-N-methylamine.

### Embodiment 15

The method of any one of embodiments 8-14, wherein the at least one compound is a source of trivalent metal, wherein the source of trivalent metal is at least one of:
(a) a compound of Formula M¹(OZ⁸)₃ (IV), wherein M¹ represents a Group 13 metal and each Z⁸ independently represents a C₁-C₆ alkyl group; or
(b) a compound of Formula (Z⁹O)₂M²-O—Si(OZ¹⁰)₃ (V), wherein M² represents a Group 13 metal and each Z⁹ and each Z¹⁰ independently represent a C₁-C₆ alkyl group.

### Embodiment 16

The method of embodiment 15, wherein the source of trivalent metal is a compound of formula (IV), wherein M¹ is Al or B and each Z⁸ independently represents a C₁-C₄ alkyl group.

### Embodiment 17

The method of embodiment 15 or 16, wherein the source of trivalent metal is a compound of formula (V), wherein M² is Al or B; and each Z⁹ and each Z¹⁰ independently represent a C₁-C₄ alkyl group.

### Embodiment 18

The method of any one of embodiments 8-16, wherein the source of a trivalent metal oxide is selected from the group consisting of aluminum trimethoxide, aluminum triethoxide, aluminum isopropoxide, and aluminum-tri-sec-butoxide.

### Embodiment 19

The method of any one of the previous embodiments, wherein the aqueous mixture comprises a base and has a pH from about 8 to about 14.

### Embodiment 20

The method of embodiment 19, wherein the base is ammonium hydroxide or a metal hydroxide.

### Embodiment 21

The method of any one of embodiments 1 to 18, wherein the aqueous mixture comprises an acid and has a pH from about 0.01 to about 6.0.

### Embodiment 22

The method of embodiment 21, wherein the acid is an inorganic acid.

### Embodiment 23

The method of embodiment 22, wherein the inorganic acid is hydrochloric acid.

### Embodiment 24

The method of any one of the previous embodiments, wherein the solution is aged in step (c) for up to 144 hours at a temperature of about 50° C. to about 200° C.

### Embodiment 25

The method of any one of the previous embodiments, wherein the pre-product is dried at a temperature of about 70° C. to about 200° C.

### Embodiment 26

The method of any one of the previous embodiments, wherein the organosilica material has an average pore diameter of about 2.0 nm to about 25.0 nm.

### Embodiment 27

The method of any one of the previous embodiments, wherein the organosilica material has a total surface area of about 200 m²/g to about 2500 m²/g.

### Embodiment 28

The method of any one of the previous embodiments, wherein the organosilica material has a pore volume of about 0.1 cm³/g to about 3.0 cm³/g.

### Embodiment 29

The method of embodiment 19 or 20, wherein the organosilica material has one or more of the following:

(i) a total surface area of about 400 m²/g to about 1700 m²/g;

(ii) a microporous surface area of about 0 m²/g to about 600 m²/g; and

(iii) a pore volume of about 0.3 cm³/g to about 3.0 cm³/g.

### Embodiment 30

The method of any one of embodiments 21-23, wherein the organosilica material has one or more of the following:

(i) a total surface area of about 200 m²/g to about 1500 m²/g;

(ii) a microporous surface area of about 100 m²/g to about 900 m²/g; and

(iii) a pore volume of about 0.1 cm³/g to about 1.0 cm³/g.

### Embodiment 31

The method of any one of embodiments 1-28, wherein the solution is aged in step (c) for about 1 hour to about 7 hours at a temperature of about 80° C. to about 100° C. and the organosilica material has one or more of the following:

(i) a total surface area of about 400 m²/g to about 1300 m²/g;

(ii) a microporous surface area of about 200 m²/g to about 600 m²/g;

(iii) a pore volume of about 0.2 cm³/g to about 0.8 cm³/g; and

(iv) an average pore radius of about 1.0 nm to about 1.5 nm.

### Embodiment 32

The method of any one of embodiments 1-28, wherein the solution is aged in step (c) for greater than about 7 hours to about 150 hours at a temperature of about 80° C. to about 100° C. and the organosilica material has one or more of the following:

(i) a total surface area of about 800 m²/g to about 1200 m²/g;

(ii) a pore volume of greater than about 0.8 cm³/g to about 1.4 cm³/g; and

(iii) an average pore radius of greater than about 1.5 nm to about 4.0 nm.

### Embodiment 33

The method of any one of embodiments 1-28, wherein the solution is aged in step (c) for about 1 hour to about 7 hours at a temperature of about 110° C. to about 130° C. and the organosilica material has one or more of the following:

(i) a pore volume of about 1.4 cm³/g to about 1.7 cm³/g; and

(ii) an average pore diameter of about 4.0 nm to about 6.0 nm.

### Embodiment 34

The method of any one of embodiments 1-28, wherein the solution is aged in step (c) for greater than about 7 hours to about 150 hours at a temperature of about 110° C. to about 130° C. and the organosilica material has one or more of the following:

(i) a pore volume of about 1.2 cm³/g to about 1.8 cm³/g; and

(ii) an average pore diameter of about 10.0 nm to about 14 nm.

### Embodiment 35

The method of any one of the previous embodiments, further comprising incorporating at least one catalytic metal within the pores of the organosilica material.

### Embodiment 36

The method of embodiment 35, wherein the catalytic metal is selected from the group consisting of a Group 6 element, a Group 8 element, a Group 9 element, a Group 10 element and a combination thereof.

### Embodiment 37

An organosilica material made according to the method of any one of embodiments 1 or 36.

### Embodiment 38

A catalyst material comprising the organosilica material of embodiment 37 and optionally, a binder.

### Embodiment 39

A method for preparing an organosilica material, the method comprising:
(a) adding a compound corresponding in structure to Formula (Ib)

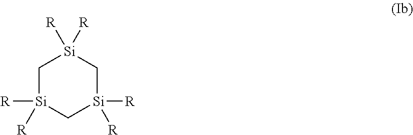

wherein each R is independently selected from the group consisting of a C₁-C₂ alkoxy and a C₁-C₂ alkyl into an aqueous mixture to form a solution;
(b) aging the solution to produce a gel; and
(c) drying the gel to obtain the organosilica material having an X-ray diffraction spectrum exhibiting substantially no peaks above 6 degrees 2θ; and wherein the method is performed using substantially no structure directing agent.

### Embodiment 40

The method of embodiment 39, wherein each R is ethoxy.

### Embodiment 41

The method of embodiment 39 or 40, wherein the organosilica material is made using substantially no added porogen.

### Embodiment 42

The method of any one of embodiments 39-41, wherein the organosilica material comprises units independently corresponding in structure to Formula (Ic)

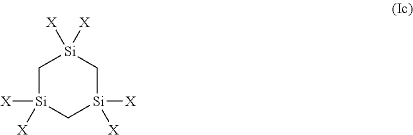

wherein each X is independently selected from the group consisting of a C₁-C₂ alkoxy, a C₁-C₂ alkyl and a hydroxyl, wherein the units are connected via at least one Si—O—Si linkage.

### Embodiment 43

The method of any one of embodiments 39-42, further comprising adding a reactant selected from the group consisting of tetraethyl orthosilicate, 1,2-bis(methyldiethoxysilyl)ethane, bis(triethoxysilyl)methane, 1,2-bis(triethoxysilyl)ethylene, 1,3,5-trimethyl-1,3,5-triethoxy-1,3,5-trisilacyclohexane, methyltriethoxysilane, and a combination thereof into the aqueous mixture to form the solution.

### Embodiment 44

A method for preparing an organosilica material, the method comprising:

(a) providing an aqueous mixture that contains essentially no structure directing agent and/or porogen,

(b) adding at least one compound of Formula R¹OR²R³R⁴Si (II),

wherein R¹ represents a C₁-C₆ alkyl group, and R², R³ and R⁴ are each independently selected from the group consisting of a C₁-C₆ alkyl group, a C₁-C₆ alkoxy group, a nitrogen-containing C₁-C₁₀ alkyl group, a nitrogen-containing heteroaralkyl group, and a nitrogen-containing optionally substituted heterocycloalkyl group;

and at least one compound of Formula Z⁵Z⁶Z⁷Si—R—SiZ⁵Z⁶Z⁷ (III), wherein

each Z⁵ represents a C₁-C₄ alkoxy group;

each Z⁶ and Z⁷ each independently represent a C₁-C₄ alkoxy group or a C₁-C₄ alkyl group; and

R is selected from the group consisting a C₁-C₈ alkylene group, a C₂-C₈ alkenylene group, a C₂-C₈ alkynylene group, a nitrogen-containing C₁-C₁₀ alkylene group, an optionally substituted C₆-C₂₀ aralkyl and an optionally substituted C₄-C₂₀ heterocycloalkyl group;

(c) aging the solution to produce a gel; and

(d) drying the gel to obtain an organosilica material which is a copolymer comprising units of Formula Z¹⁵Z¹⁶Z¹⁷Si—R⁵—SiZ¹⁵Z¹⁶Z¹⁷ (VII), wherein each Z¹⁵ can be a hydroxyl group, a C₁-C₄ alkoxy group or an oxygen bonded to a silicon atom of another comonomer; each Z¹⁶ and Z¹⁷ independently can be a hydroxyl group, a C₁-C₄ alkoxy group, a C₁-C₄ alkyl group or an oxygen bonded to a silicon atom of another monomer; and R⁵ can be selected from the group consisting of a C₁-C₈ alkylene group, a C₂-C₈ alkenylene group, a C₂-C₈ alkynylene group, a nitrogen-containing C₁-C₁₀ alkylene group, an optionally substituted C₆-C₂₀ aralkyl and an optionally substituted C₄-C₂₀ heterocycloalkyl group; and units of Formula Z¹¹OZ¹²Z¹³Z¹⁴ (VI), wherein Z¹¹ can be a hydrogen atom or a C₁-C₄ alkyl group or a bond to a silicon atom of another monomer; and Z¹², Z¹³ and Z¹⁴ each independently can be selected from the group consisting of a hydroxyl group, a C₁-C₄ alkyl group, a C₁-C₄ alkoxy group, a nitrogen-containing C₁-C₁₀ alkyl group, a nitrogen-containing heteroalkyl group, a nitrogen-containing optionally substituted heterocycloalkyl group and an oxygen atom bonded to a silicon atom of another monomer.

### Embodiment 45

The method of embodiment 44, wherein, in the compound of Formula Z⁵Z⁶Z⁷Si—R⁵—SiZ⁵Z⁶Z⁷ (III), each Z⁵ represents a C₁-C₄ alkoxy group; each Z⁶ and Z⁷ independently represent a C₁-C₄ alkoxy group or a C₁-C₄ alkyl group; and R⁵ is methylene or ethylene and as the compound of Formula (II), a tetralkyl orthosilicate is used to produce an organosilica material which is a copolymer comprising: units of Formula Z¹⁵Z¹⁶Z¹⁷Si—R⁵—SiZ¹⁵Z¹⁶Z¹⁷ (VII), wherein each Z¹⁵ can be a hydroxyl group, a C₁-C₄ alkoxy group or an oxygen bonded to a silicon atom of another comonomer; each Z¹⁶ and Z¹⁷ independently can be a hydroxyl group, a C₁-C₄ alkoxy group, a C₁-C₄ alkyl group or an oxygen bonded to a silicon atom of another monomer; and R⁵ is a methylene or ethylene group; and units of Formula Z¹¹OZ¹²Z¹³Z¹⁴ (VI), wherein Z¹¹ can be a hydrogen atom or a C₁-C₄ alkyl group or a bond to a silicon atom of another monomer; and Z¹², Z¹³ and Z¹⁴ each independently can be selected from the group consisting of a hydroxyl group, a C₁-C₄ alkoxy group and an oxygen atom bonded to a silicon atom of another monomer.

### Embodiment 46

The method of embodiment 45, wherein the compound of Formula (III) is (bis(triethoxysilyl)methane and the compound of Formula (II) is tetraethyl orthosilicate (TEOS) and the organosilica material made is a copolymer comprising: units of Formula Z¹⁵Z¹⁶Z¹⁷Si—R⁵—SiZ¹⁵Z¹⁶Z¹⁷ (VII), wherein each Z¹⁵, and Z¹⁷ independently can be a hydroxyl group, an ethoxy group or an oxygen bonded to a silicon atom of another comonomer; and R⁵ is a methylene group; and units of Formula Z¹¹OZ¹²Z¹³Z¹⁴ (VI), wherein Z¹¹ can be a hydrogen atom or an ethyl group or a bond to a silicon atom of another monomer; and Z¹², Z¹³ and Z¹⁴ each independently can be selected from the group consisting of a hydroxyl group, an ethoxy group and an oxygen atom bonded to a silicon atom of another monomer.

### EXAMPLES

The following examples are merely illustrative, and do not limit this disclosure in any way.

### General Methods

### Small Angle X-Ray Diffraction Analysis

X-ray powder diffraction (XRD) patterns were collected on a PANalytical X'pert diffractometer equipped with an accessory for low angle measurements. XRD analyses were recorded using the Cu Ka (=1.5405980 Å) line in the 2θ range from 0.5 to 10° with a step size of 0.0167° and a counting time of 1.2 s.

### Solid-State (SS) NMR Measurements

The ²⁹Si MAS NMR spectra were recorded on a Varian InfinityPlus-400 spectrometer (operating at 9.4 T) and Varian InfinityPlus-500 (operating at 11.74 T), corresponding to ²⁹Si Larmor frequencies of 79.4 MHz and 99.2 MHz, respectively, with a 7.5 mm MAS probe heads using 5 kHz spinning, 4.0 μs 90° pulses, and at least 60 s recycle delay, with proton decoupling during data acquisition. The ²⁹Si chemical shifts are referenced with respect to an external tetramethyl silane (δ_{Si}=0.0 ppm). The ¹³C CPMAS NMR spectra were recorded on a Varian InfinityPlus-500 spectrometer corresponding to ¹³C Larmor frequency of 125 MHz, with 1.6 mm MAS probe head using 40 kHz spinning, ¹H-¹³C cross-polarization (CP) contact time of at least 1 ms, a recycle delay of at least 1 s, with proton decoupling during data acquisition. The ¹³C chemical shifts are referenced with respect to an external tetramethyl silane (δ_{C}=0.0 ppm). The ²⁷Al MAS NMR spectra were recorded on a Varian InfinityPlus-500 corresponding to ²⁷Al Larmor frequency of 130.1 MHz using a 4 mm MAS probe head using 12 kHz spinning, with a π/12 radian pulse length, with proton decoupling during data acquisition, and a recycle delay of 0.3 s. The chemical shifts are referenced with respect to an external solution of Al(H₂O)₆³⁺ (δ_{Al}=0.0 ppm). All NMR spectra were recorded at room temperature using air for spinning.

### Thermal Gravimetric Analysis (TGA)

Thermal stability results were recorded on Q5000 TGA. Ramp rate was 5° C./min, temperature range was from 25° C. to 800° C. All the samples were tested in both air and nitrogen.

### CO₂ Adsorption

The work was done with a Quantchrom autosorb iQ2. All the samples were pre-treated at 120° C. in vacuum for 3 hours before collecting the CO₂ isotherm at different temperatures.

### Nitrogen Porosimetry

The nitrogen adsorption/desorption analyses was performed with different instruments, e.g. TriStar 3000, TriStar II 3020 and Autosorb-1. All the samples were pre-treated at 120° C. in vacuum for 4 hours before collecting the N₂ isotherm. The analysis program calculated the experimental data and report BET surface area (total surface area), microporous surface area (S), total pore volume, pore volume for micropores, average pore diameter (or radius), etc.

### Example 1

### Organosilica Material Syntheses Using Formula [Z¹Z²SiCH₂]₃ (Ia) in Basic or Acidic Media

### 1A. Synthesis Using [(EtO)₂SiCH₂]₃ in Basic Aqueous Medium—Without Surfactant

A solution with 18.6 g of 30% NH₄OH and 23.76 g deionized water (DI) water was made. The pH of the solution was 12.55. To the solution, 3.0 g of 1,1,3,3,5,5-hexaethoxy-1,3,5-trisilacyclohexane ([(EtO)₂SiCH₂]₃) was added, producing a mixture having the molar composition:

1.0[(EtO)₂SiCH₂]₃:21OH:270H₂O

and stirred for 1 day at room temperature (20-25° C.). The solution was transferred to an autoclave and aged at 80° C.-90° C. for 1 day to produce a gel. The gel was dried at 80° C. in a vacuum to remove most of the water and then fully dried at 110° C. for three hours. This produced Sample 1A as a clear solid, which was converted to white powder after grinding. No surface directing agent or porogen were used in this preparation.

The procedure was repeated with the following molar composition

4.0[(EtO)₂SiCH₂]₃:21OH:270H₂O

to produce Sample 1B.

XRD Analysis

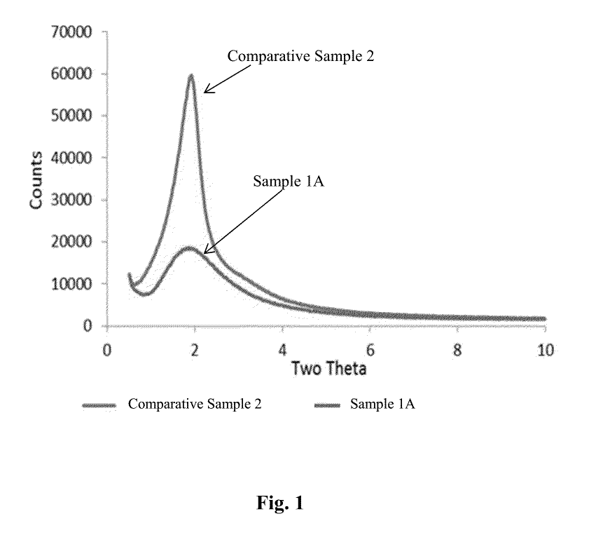

XRD was performed on Sample 1A. The XRD pattern of Sample 1A is shown in FIG. 1.

TGA Analysis

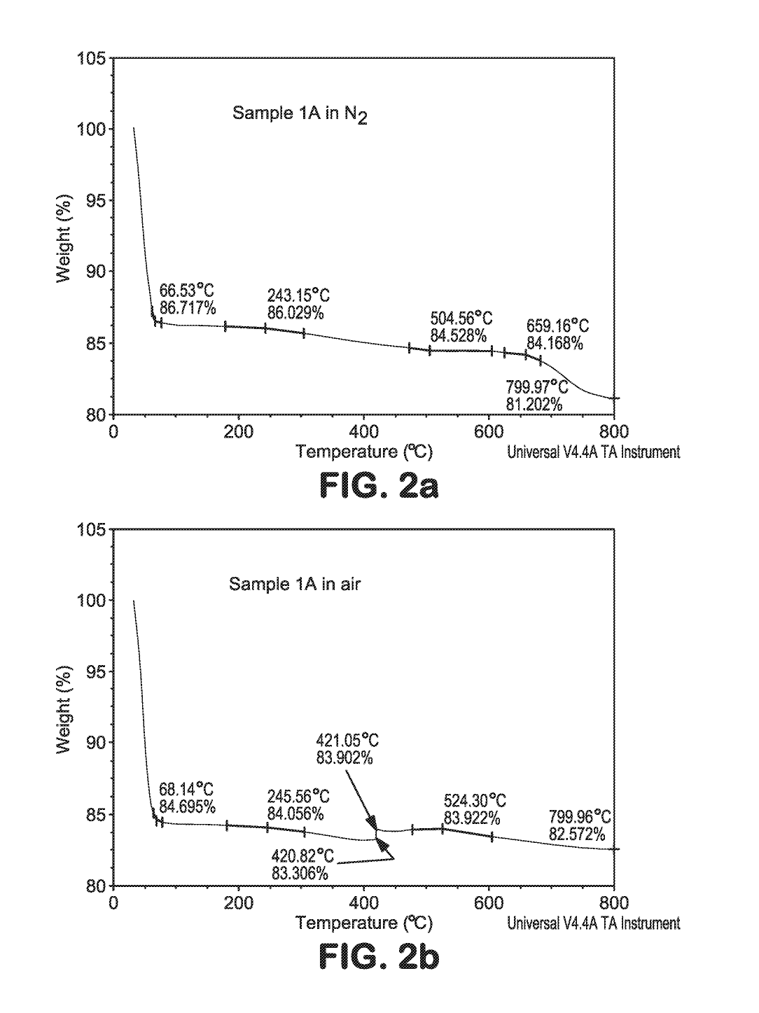

TGA weight loss studies were performed on Sample 1A in nitrogen and air. FIGS. 2a and 2b display the TGA data for Sample 1A in nitrogen and air, respectively.

Nitrogen Adsorption/Desorption Analysis

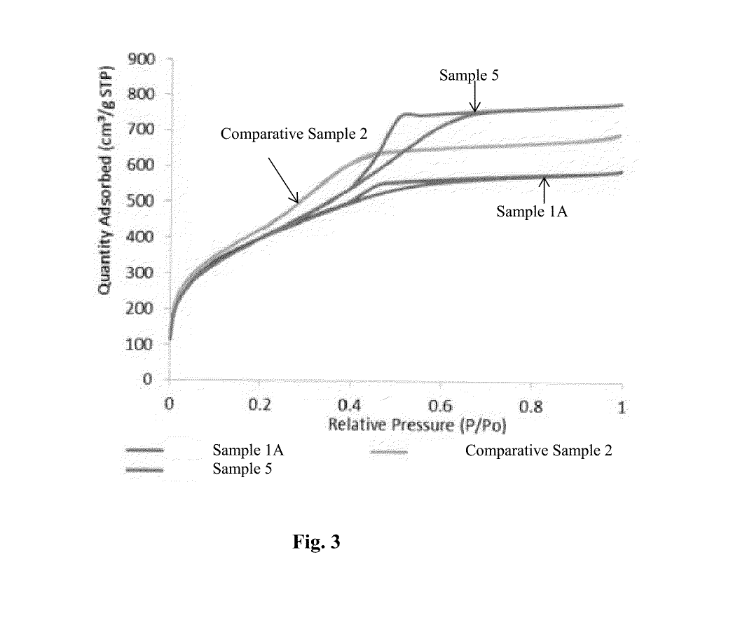

Nitrogen adsorption/desorption analysis was performed on Sample 1A, and the results are provided in Table 1 below and FIGS. 3-6.

SS-NMR-Analysis

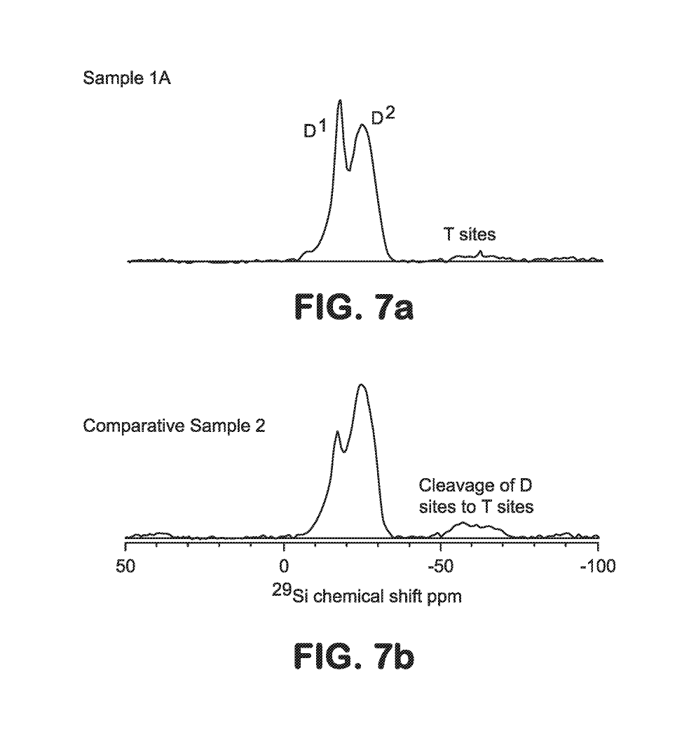

Sample 1A was characterized with ²⁹Si MAS NMR with the results as shown in FIG. 7a.

### 1B. Comparative—Synthesis Using [(EtO)₂SiCH₂]₃ in Basic Aqueous Medium—with Surfactant

In this example, an organosilica material was prepared according to Landskron, K., et al., Science 302:266-269 (2003).

Cetyltrimethylammonium bromide (CTMABr, 0.9 mmol, 0.32 g, Aldrich) was dissolved in a mixture of 2.16 g NH₄OH (35 wt %) and 3.96 g de-ionized water at 20° C. to form a solution.

[(EtO)₂SiCH₂]₃ (1.26 mmol, 0.5 g) was added to the solution, producing a solution having the molar composition:

1.0[(EtO)₂SiCH₂]₃:17OH:236H₂O:0.7CTMABr

which was stirred for 1 day at 20° C. and a white precipitate formed. Afterwards, the solution was aged for 1 day at 80° C. Then the precipitate was filtered off and washed with water. The sample was then stirred for 48 hours in a solution of 12 g HCl (36 wt %) and 80 g of methanol. The sample was then filtered off again and washed with MeOH, resulting in Comparative Sample 2.

XRD Analysis

XRD was performed Comparative Sample 2. A comparison of the XRD patterns for Sample A1 and Comparative Sample 2 is shown in FIG. 1. Compared to the XRD pattern of Sample 1A, the XRD pattern of Comparative Sample 2 exhibits a shoulder at about 3 degrees 2θ.

TGA Analysis

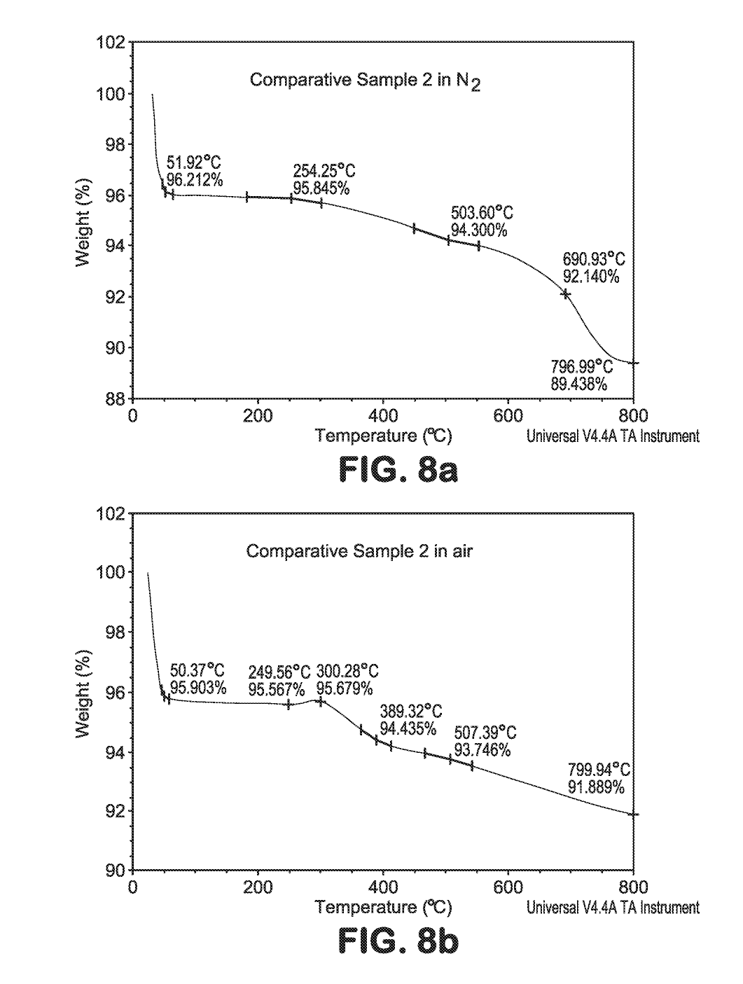

TGA weight loss studies were performed on Comparative Sample 2 in nitrogen and air. FIGS. 8a and 8b display the TGA data for Comparative Sample 2 in nitrogen and air, respectively.

Nitrogen Adsorption/Desorption Analysis

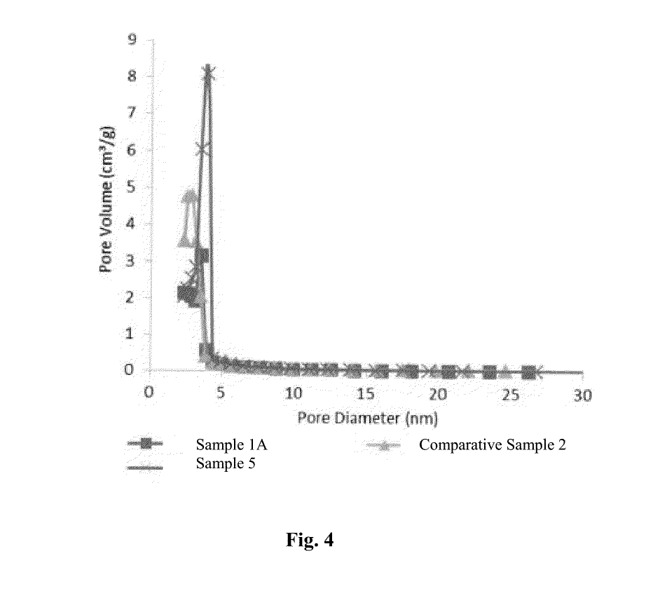

Nitrogen adsorption/desorption analysis was performed on Comparative Sample 2. The surface area, average pore diameter, and pore volume obtained by the nitrogen adsorption/desorption analysis for Sample 1A and Comparative Sample 2 are shown below in Table 1 and FIGS. 3 and 4.

TABLE 1

| | BET | Pore Diameter | Pore Volume |
|---|---|---|---|
| Material | (m²/g) | (nm) | (cc/g) |
| Comparative Sample 2 | 1520 | 3.02 | 1.07 |
| Sample 1A | 1410 | 3.18 | 0.92 |

SS-NMR-Analysis

Comparative Sample 2 was characterized with ²⁹Si MAS NMR as shown in FIG. 7b. As shown below in Table 2, Sample 1A had a higher silanol content (i.e., 47%) compared to Comparative Sample 2 (i.e., 41%).

TABLE 2

| | | | T | |
|---|---|---|---|---|
| | D₁ | D₂ | sites | Si(OH)/Si |
| Sample 1A (%) | 96 | | 4 | 47 |
| | 45.6 | 50.4 | | |
| Comparative | 89 | | 11 | 41 |
| Sample 2(%) | 34.7 | 54.3 | | |

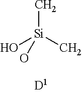

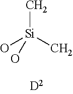

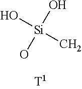

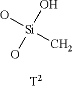

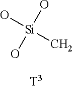

### 1C. Synthesis Using [(EtO)₂SiCH₂]₃ in Acidic Aqueous Medium—without Surfactant

A 14 g HCl solution with a pH of 2 was made by adding 0.778 mol water and 0.14 mmol HCl. To the solution, 1.0 g (2.52 mmol) of [(EtO)₂SiCH₂]₃ was added producing a solution having the molar composition:

18[(EtO)₂SiCH₂]₃:1HCl:5556H₂O

which was stirred for 1 day at room temperature (20-25° C.). The solution was transferred to an autoclave and aged at 94° C. for 1 day to produce a gel. The gel was dried in a vacuum at 120° C. overnight (16-24 hours) to produce Sample 3. No surface directing agent or porogen were used.

XRD Analysis

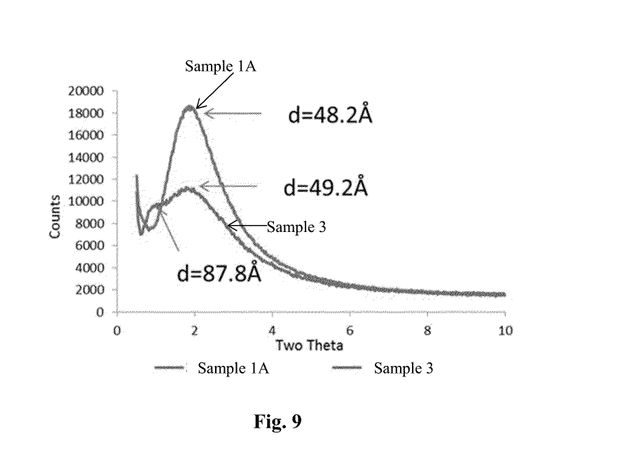

XRD was performed on Sample 3. A comparison of XRD patterns for Sample 1A and Sample 3 is shown in FIG. 9.

Nitrogen Adsorption/Desorption Analysis

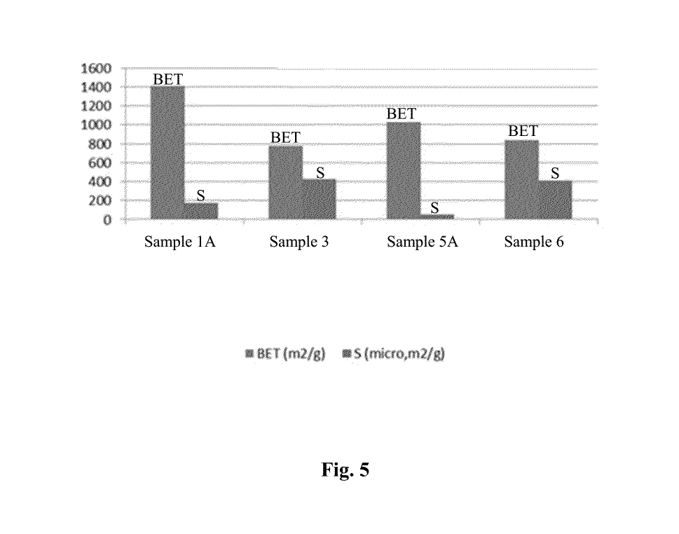

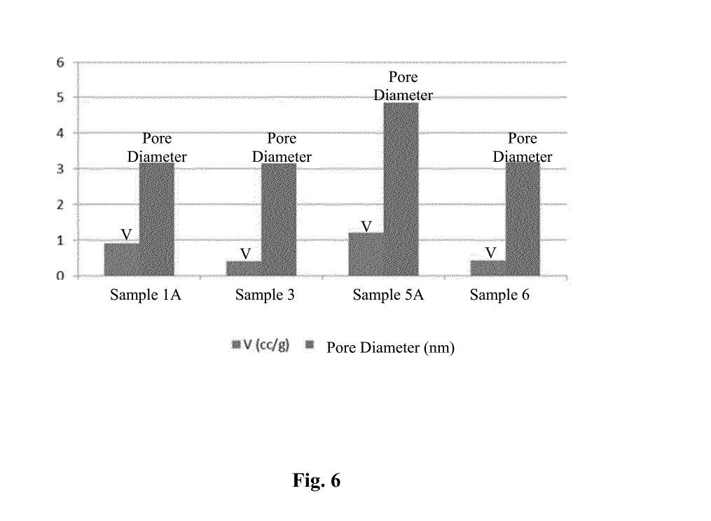

Nitrogen adsorption/desorption analysis was performed on Sample 3. The surface area, microporous surface area, average pore diameter, and pore volume obtained by the nitrogen adsorption/desorption analysis for Sample 3 are shown in FIGS. 5 and 6.

### 1D. Synthesis Using [(EtO)₂SiCH₂]₃ and [CH₃EtOSiCH₂]₃

A solution with 6.21 g of 30% NH₄OH and 7.92 g DI water was made. To the solution, 0.6 g of [(EtO)₂SiCH₂]₃ and 0.306 g of 1,3,5-trimethyl-1,3,5-triethoxy-1,3,5-trisilacyclohexane ([CH₃EtOSiCH₂]₃) was added producing a solution having the molar composition:

1.5[(EtO)₂SiCH₂]₃:1.0[CH₃EtOSiCH₂]₃:53OH:682H₂O

which was stirred for 1 day at room temperature (20-25° C.). The solution was transferred to an autoclave and aged at 90° C. for 1 day to produce a gel. The gel was dried in a vacuum at 120° C. overnight (16-24 hours) and Sample 4A was obtained. No structure directing agent or porogen were used.

Nitrogen Adsorption/Desorption Analysis

This above preparation method was repeated, except the relative ratio of [(EtO)₂SiCH₂]₃ (Reagent 1) to [CH₃EtOSiCH₂]₃ (Reagent 2) was varied. Nitrogen adsorption/desorption analysis was performed on each material and the results for each to material is given below in Table 3.

TABLE 3

| | | BET | | Pore Diameter |
|---|---|---|---|---|
| Material | Reagent 1:Reagent 2 | (m²/g) | V (cc/g) | (nm) |
| Sample 1A | 5:0 | 1410 | 0.915 | 3.18 |
| Sample 4A | 3:2 | 819 | 1.52 | 7.39 |
| Sample 4B | 4:1 | 1100 | 1.14 | 4.17 |
| Sample 4C | 2:3 | 460 | 1.09 | 13.9 |
| Sample 4D | 0:5 | 1.81 | 7.73E−03 | 68.8 |

As Reagent 2 increased, the average pore diameter was observed to increase, which without being bound by theory may be due to Reagent 2 containing less reactive —OR groups compared to Reagent 1. The porosity of the material decreased as Reagent 2 was greater than 60% (mol ratio).

SS-NMR-Analysis

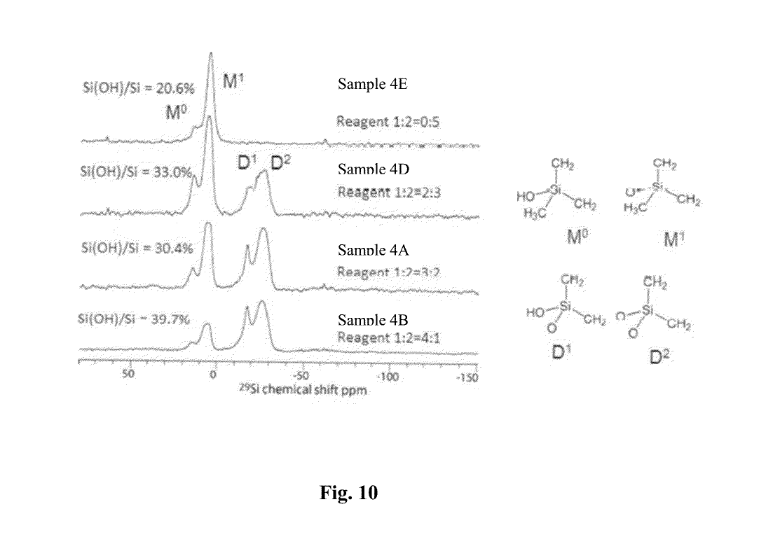

The materials in Table 3 were characterized with ²⁹Si MAS NMR, as shown in FIG. 10.

### Example 2

### Organosilica Material Syntheses Using Formula [Z¹Z²SiCH₂]₃ (Ia) and Formula R¹OR²R³R⁴Si (II) in Basic or Acidic Media

### 2A. Synthesis Using [(EtO)₂SiCH₂]₃ and Tetraethylorthosilicate (TEOS) ((EtO)₄Si) in Basic Aqueous Medium

A solution with 6.21 g of 30% NH₄OH (53 mmol NH₄OH) and 7.92 g DI water was made. To the solution, 0.8 g (2 mmol) of [(EtO)₂SiCH₂]₃ and 0.625 g (3 mmol) of TEOS was added to produce a solution having the molar composition:

2.0[(EtO)₂SiCH₂]₃:3.0TEOS:53OH:682H₂O

which was stirred for three days at room temperature (20-25° C.). The solution was transferred to an autoclave and aged at 80° C.-90° C. for 2 days to produce a gel. The gel was dried in a vacuum at 110° C. overnight (16-24 hours) and Sample 5 was obtained. No structure directing agent or porogen was used.

A solution with 6.21 g of 30% NH₄OH (53 mmol NH₄OH) and 7.92 g DI water was made. To the solution, 3.2 g (8 mmol) of [(EtO)₂SiCH₂]₃ and 2.5 g (12 mmol) of TEOS was added to produce a solution having the molar composition:

8.0[(EtO)₂SiCH₂]₃:12.0TEOS:53OH:682H₂O

which was stirred for three days at room temperature (20-25° C.). The solution was transferred to an autoclave and aged at 80° C.-90° C. for 2 days to produce a gel. The gel was dried in a vacuum at 110° C. overnight (16-24 hours) and Sample 5A was obtained. No structure directing agent or porogen was used.

XRD Analysis

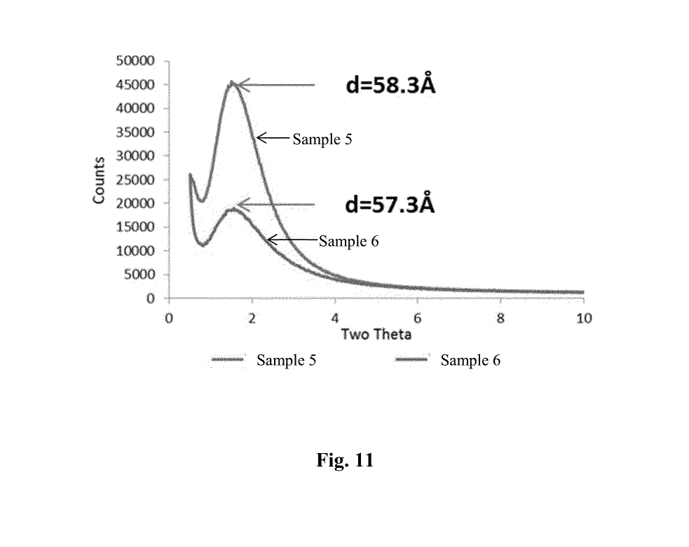

XRD was performed on Sample 5. The XRD pattern of Sample 5 is shown in FIG. 11.

TGA Analysis

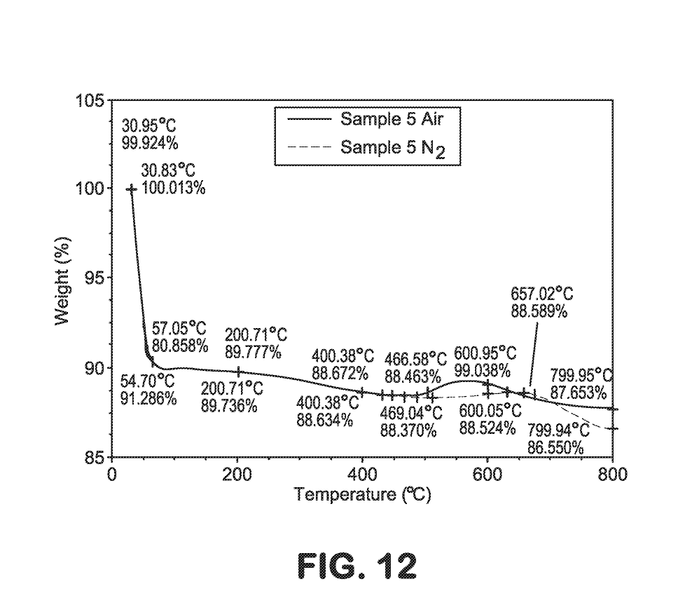

TGA weight loss studies were performed on Sample 5 in nitrogen and air. FIG. 12 display the TGA data for Sample 1A in nitrogen and air.

SS-NMR-Analysis

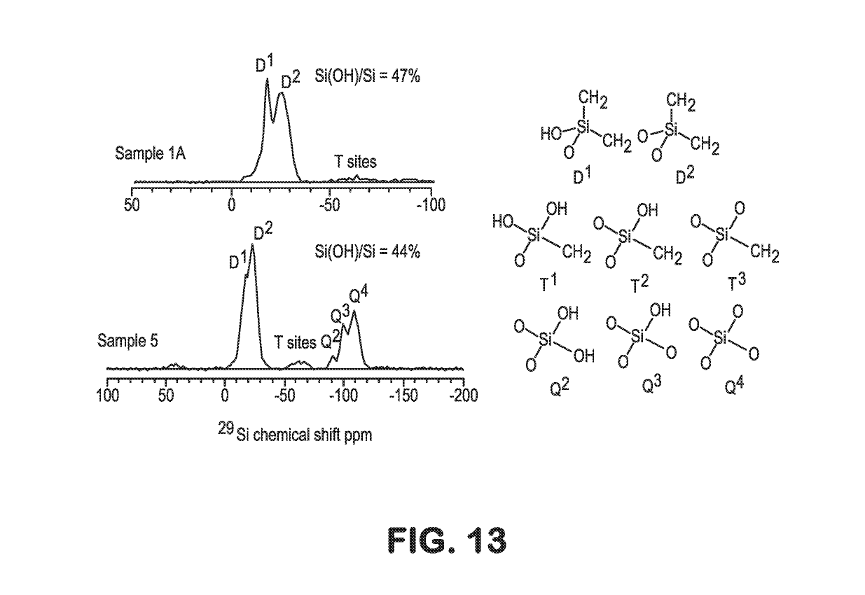

Sample 5 was characterized with ²⁹Si MAS NMR and compared with Sample 1A as shown in FIG. 13. As shown in FIG. 13, Sample 5 had a silanol content of 44%.

Nitrogen Adsorption/Desorption Analysis

Nitrogen adsorption/desorption analysis was performed on Sample 5, and the results are provided below in Table 4 and FIGS. 4, 5 and 6.

TABLE 4

| | BET | Pore Diameter | |
|---|---|---|---|
| Material | (m²/g) | (nm) | Pore Volume (cc/g) |
| Sample 5 | 1430 | 3.42 | 1.21 |
| Sample 5A | 1027 | 4.84 | 1.20 |

### 2B. Synthesis Using [(EtO)₂SiCH₂]₃ and TEOS in Acidic Aqueous Medium

A 14 g HCl solution with a pH of 2 was made by adding 0.778 mol water and 0.14 mmol HCl. To the solution, 0.8 g (2 mmol) of [(EtO)₂SiCH₂]₃ and 0.625 g (3 mmol) TEOS was added to produce a solution having the molar composition:

2.0[(EtO)₂SiCH₂]₃:3.0TEOS:0.14H:778H₂O

which was stirred for 1 day at room temperature (20-25° C.). The solution was transferred to an autoclave and aged at 94° C. for 1 day to produce a gel. The gel was dried in a vacuum at 120° C. overnight (16-24 hours) to produce Sample 6. No structure directing agent or porogen were used.

XRD Analysis

XRD was performed on Sample 6. The XRD pattern of Sample 6 is shown in FIG. 11.

Nitrogen Adsorption/Desorption Analysis

Nitrogen adsorption/desorption analysis was performed on Sample 6, and the results are provided in FIGS. 5 and 6.

### 2C. Synthesis Using [CH₃EtOSiCH₂]₃ and TEOS

A solution with 6.21 g of 30% NH₄OH (53 mmol NH₄OH) and 7.92 g DI water was made. To the solution, 0.612 g (2 mmol) of 1,3,5-trimethyl-1,3,5-triethoxy-1,3,5-trisilacyclohexane ([CH₃EtOSiCH₂]₃) and 0.625 g (3 mmoles) of TEOS was added to produce a solution having the molar composition:

2.0[CH₃EtOSiCH₂]₃:3.0TEOS:53OH:682H₂O

which was stirred for 1 day at room temperature (20-25° C.). The solution was transferred to an autoclave and aged at 90° C. for 1 day to produce a gel. The gel was dried in a vacuum at 120° C. overnight (16-24 hours) and Sample 7A was obtained. No structure directing agent or porogen were used.

Nitrogen Adsorption/Desorption Analysis

This above preparation method was repeated, except the relative ratio of TEOS (Reagent 3) to [CH₃EtOSiCH₂]₃ (Reagent 2) was varied. Table 5 below is a summary of the N₂ adsorption analysis for the materials obtained with varied reagent ratios.

TABLE 5

| | | BET | Pore Volume | Pore Diameter |
|---|---|---|---|---|
| Material | (Reagent 3:Reagent 2) | (m²/g) | (cc/g) | (nm) |
| Sample 7A | 3:2 | 471 | 1.9 | 18.6 |
| Sample 7B | 3:4 | 493 | 2.16 | 23.1 |

SS-NMR-Analysis

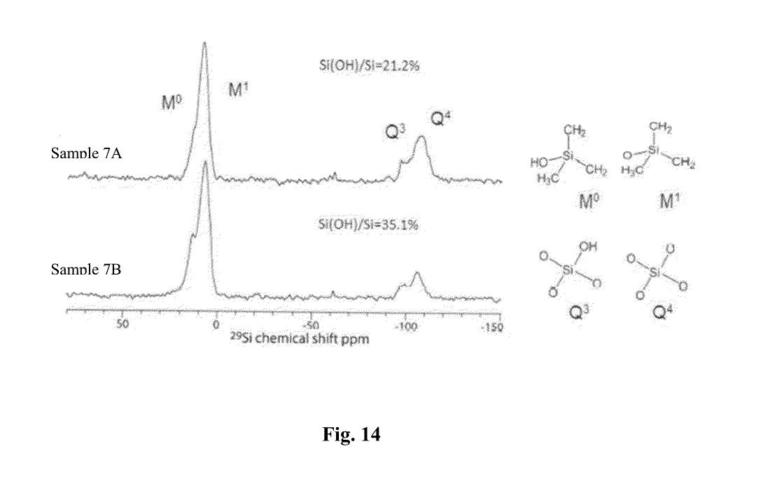

The materials made by this method were characterized with by ²⁹Si MAS NMR, as shown in FIG. 14.

### 2D. Synthesis Using [(EtO)₂SiCH₂]₃ and methyltriethoxysilane (MTES) ((EtO)₃CH₃Si)

A solution with 6.21 g of 30% NH₄OH (53 mmol NH₄OH) and 7.92 g DI water was made. To the solution, 0.4 g (1 mmol) of [(EtO)₂SiCH₂]₃ and 0.267 g (1.5 mmol) of MTES was added to produce a solution having the molar composition:

1.0[(EtO)₂SiCH₂]₃:1.5MTES:53OH:682H₂O

which was stirred for 1 day at room temperature (20-25° C.). The solution was transferred to an autoclave and aged at 90° C. for 1 day to produce a gel. The gel was dried in a vacuum at 120° C. overnight (16-24 hours) and Sample 8A was obtained. No structure directing agent or porogen were used.

Nitrogen Adsorption/Desorption Analysis

This above preparation method was repeated, except the relative ratio of [(EtO)₂SiCH₂]₃ (Reagent 1) and of MTES (Reagent 2) was varied. Table 6 below is a summary of the N₂ adsorption analysis for the materials obtained with varied reagent ratios.

TABLE 6

| | | BET | Pore Volume | Pore Diameter |
|---|---|---|---|---|
| Material | Reagent 1:Reagent 2 | (m²/g) | (cc/g) | (nm) |
| Sample 1A | 5:0 | 1410 | 0.915 | 3.18 |
| Sample 8A | 2:3 | 821 | 1.06 | 4.5 |
| Sample 8B | 4:1 | 1130 | 1.0 | 3.59 |
| Sample 8C | 3:2 | 1040 | 1.05 | 3.89 |

### Example 3

### Organosilica Material Syntheses Using Formula [Z¹Z²SiCH₂]₃ (Ia) Formula R¹OR²R³R⁴Si (II), and/or Formula Z⁵Z⁶Z⁷Si—R—SiZ⁵Z⁶Z⁷ (III)

### 3A. Synthesis Using [(EtO)₂SiCH₂]₃ and CH₃(EtO)₂Si—CH₂CH₂—Si(EtO)₂CH₃

A solution with 6.21 g of 30% NH₄OH (53 mmol NH₄OH) and 7.9 g DI water was made. To the solution, 0.8 g (2 mmol) of [(EtO)₂SiCH₂]₃ and 0.88 g (3 mmol) 1,2-bis(methyldiethyoxysilyl)ethane (CH₃(EtO)₂Si—CH₂CH₂—Si(EtO)₂CH₃) was added to produce a solution having the molar composition:

2.0[(EtO)₂SiCH₂]₃:3.0CH₃(EtO)₂Si—CH₂CH₂—Si(EtO)₂CH₃:53OH:682H₂O

which was stirred for 1 day at room temperature (20-25° C.). The solution was transferred to an autoclave and aged at 80° C.-90° C. for 1 day to produce a gel. The gel was dried in a vacuum at 110° C. overnight (16-24 hours) and Sample 9 was obtained. No structure directing agent or porogen were used.

XRD Analysis

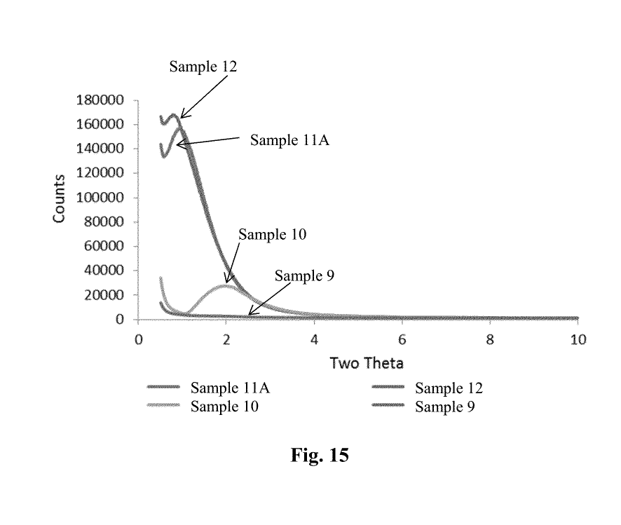

XRD was performed on Sample 9. The XRD pattern of Sample 9 is shown in FIG. 15.

Nitrogen Adsorption/Desorption Analysis

Nitrogen adsorption/desorption analysis was performed on Sample 9, and the results are provided in Table 7.

### 3B. Synthesis Using [(EtO)₂SiCH₂]₃ and (EtO)₃Si—CH₂—Si(EtO)₃

A solution with 6.21 g of 30% NH₄OH (53 mmol NH₄OH) and 7.9 g DI water was made. To the solution, 0.8 g (2 mmol) of [(EtO)₂SiCH₂]₃ and 1.02 g (3 mmol) of bis(triethoxysilyl)methane ((EtO)₃Si—CH₂—Si(EtO)₃) was added to produce a solution having the molar composition:

2.0[(EtO)₂SiCH₂]₃:3.0(EtO)₃Si—CH₂—Si(EtO)₃:53OH:682H₂O

which was stirred for 1 day at room temperature (20-25° C.). The solution was transferred to an autoclave and aged at 80° C.-90° C. for 1 day to produce a gel. The gel was dried in a vacuum at 110° C. overnight (16-24 hours) and Sample 10 was obtained. No structure directing agent or porogen were used.

XRD Analysis

XRD was performed on Sample 10. The XRD pattern of Sample 10 is shown in FIG. 15.

Nitrogen Adsorption/Desorption Analysis

Nitrogen adsorption/desorption analysis was performed on Sample 10, and the results are provided in Table 7.

### 3C. Synthesis Using TEOS and (EtO)₃Si—CH₂—Si(EtO)₃

A solution with 6.21 g of 30% NH₄OH (53 mmoles NH₄OH) and 7.92 g DI water was made. To the solution, 1.7 g (5 mmol) of bis(triethoxysilyl)methane ((EtO)₃Si—CH₂—Si(EtO)₃) and 0.416 g (2 mmol) of TEOS were added to produce a solution having the molar composition:

5.0(EtO)₃Si—CH₂—Si(EtO)₃:2.0TEOS:53OH:682H₂O

which was stirred for 1 day at room temperature (20-25° C.). The solution was transferred to an autoclave and aged at 80° C.-90° C. for 1 day to produce a gel. The gel was dried in a vacuum at 110° C. overnight (8-16 hours) and Sample 11A was obtained. No structure directing agent or porogen were used.

Two more preparations with different ratios of reagents were also made, one with a (EtO)₃Si—CH₂—Si(EtO)₃:TEOS molar ratio of 4:4 to obtain Sample 11B and another with a (EtO)₃Si—CH₂—Si(EtO)₃:TEOS molar ratio of 3:6 to obtain Sample 11C.

XRD Analysis

XRD was performed on Sample 11A. The XRD pattern of Sample 11A is shown in FIG. 15.

Nitrogen Adsorption/Desorption Analysis

Nitrogen adsorption/desorption analysis was performed on Sample 11A, and the results are provided in Table 7.

### 3D. Synthesis Using [(EtO)₂SiCH₂]₃ and (EtO)₃Si—CH═CH—Si(EtO)₃

A solution with 12.42 g of 30% NH₄OH (106 mmol NH₄OH) and 15.8 g DI water was made. To the solution, 1.6 g (4 mmol) of [(EtO)₂SiCH₂]₃ and 0.352 g (1 mmol) 1,2-bis(triethoxysilyl)ethylene ((EtO)₃Si—CH═CH—Si(EtO)₃) was added to produce a solution having the molar composition:

4.0[(EtO)₂SiCH₂]₃:1.0(EtO)₃Si—CH═CH—Si(EtO)₃:106OH:1364H₂O

which was stirred for 1 day at room temperature (20-25° C.). The solution was transferred to an autoclave and aged at 80° C.-90° C. for 1 day to produce a gel. The gel was dried in a vacuum at 110° C. overnight (8-16 hours) and Sample 12 was obtained. No structure directing agent or porogen were used.

XRD Analysis

XRD was performed on Sample 12. The XRD pattern of Sample 12 is shown in FIG. 15.

Nitrogen Adsorption/Desorption Analysis

Nitrogen adsorption/desorption analysis was performed on Sample 12, and the results are provided in Table 7.

TABLE 7

| | BET | S (m2/g, | Pore Diameter | Pore Volume |
|---|---|---|---|---|
| Material | (m2/g) | micro) | (nm) | (cc/g) |
| Sample 9 | 551 | 233 | 8.4 | 0.76 |
| Sample 10 | 1270 | 512 | 3.35 | 0.96 |
| Sample 11A | 870 | 0 | 3.83 | 0.84 |
| Sample 12 | 1030 | 0 | 3.69 | 1.02 |

### Example 4

### Organosilica Material Syntheses Using Formula [Z¹Z²SiCH₂]₃ (Ia) and Nitrogen-Containing Monomers

Synthesis:
1. Made a solution with 6.21 g 30% NH₄OH and 7.9 g DI water (53 mmol NH₄OH; 682 mmol H₂O);
2. Added 0.8 g (2 mmol) of [(EtO)₂SiCH₂]₃ (Reagent 1) to Reagent 2 into the above solution, kept stirring for 1 day at room temperature;
3. Transferred the solution to an autoclave, aging at 80-90° C. for 1 day;
4. Dried the gel at 110° C. in vacuum overnight.

The above synthesis was performed with the following reagents in Table 8 to obtain Samples 13, 14, 15 and 21.

The above synthesis was performed with the following reagents in Table 8 to obtain Samples 16, 17, 18 and 19 except 1.6 g of Reagent 1, 12.4 g 30% NH₄OH and 15.8 g DI water were used for the preparation.

The above synthesis was performed with the following reagents in Table 8 to obtain Sample 21 except 3.2 g of Reagent 1, 24.8 g 30% NH₄OH and 31.6 g of DI water were used for the preparation.

TABLE 8

| | | Reagent 2 | Reagent 1: Reagent 2 |
|---|---|---|---|
| Material | Reagent 2 | Amount (g) | Molar ratio |
| Sample 13 | N,N′-bis[(3- | 0.192 | 2:0.5 |
| | trimethoxysilyl)propyl]ethylenediamine | | |
| Sample 14 | bis[(methyldiethoxysilyl)propyl]amine | 0.183 | 2:0.5 |
| Sample 15 | bis[(methyldimethoxysilyl)propyl]-N- | 0.162 | 2:0.5 |
| | methylamine | | |
| Sample 16 | (N,N-dimethylaminopropyl)trimethoxysilane | 1.24 | 2:3 |
| Sample 17 | N-(2-aminoethyl)-3- | 1.58 | 2:3 |
| | aminopropyltriethoxysilane | | |
| Sample 18 | 4-methyl-1-(3-triethoxysilylpropyl)-piperazine | 1.83 | 2:3 |
| Sample 19 | 4-(2-(triethoxysily)ethyl)pyridine | 0.271 | 2:0.5 |
| Sample 20 | 1-(3-(triethoxysilyl)propyl)-4,5-dihydro-1H- | 0.553 | 2:0.5 |
| | imidazole | | |
| Sample 21 | (3-aminopropyl)triethoxysilane | 0.22 | 2:0.5 |

XRD Analysis

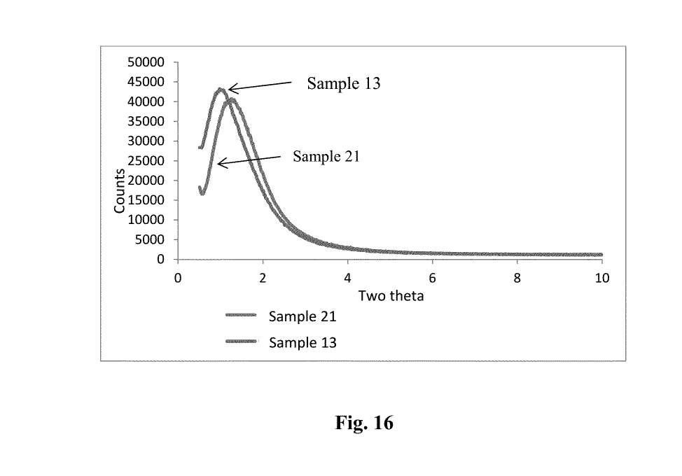

XRD was performed on Samples 13 and 21. The XRD patterns of Samples 13 and 21 are shown in FIG. 16.

Nitrogen Adsorption/Desorption Analysis

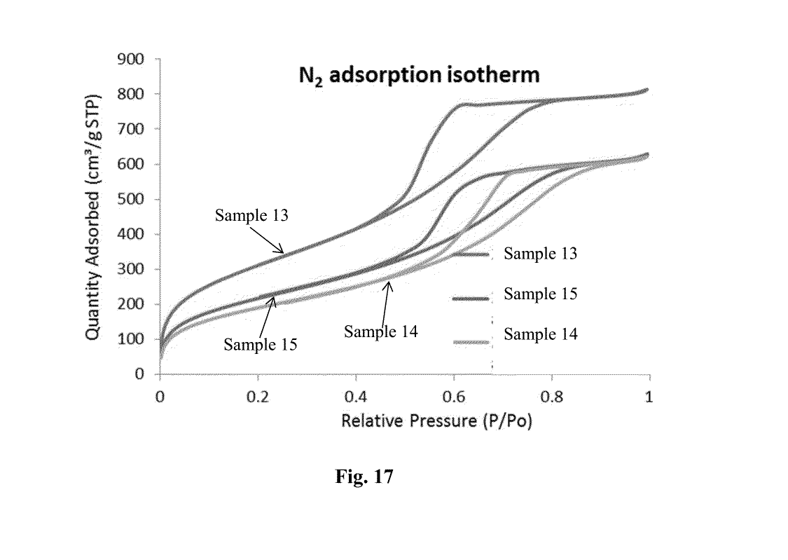

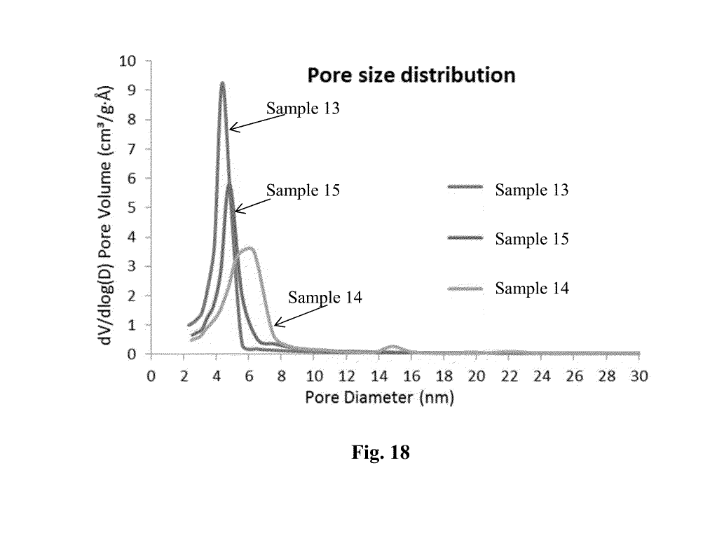

Nitrogen adsorption/desorption analysis was performed on Samples 13, 14 and 15, and the results are provided in Table 9 and FIGS. 17 and 18.

TABLE 9

| | BET | Pore Diameter | Pore Volume |
|---|---|---|---|
| Material | (m²/g) | (nm) | (cc/g) |
| Sample 13 | 1127 | 4.11 | 1.26 |
| Sample 14 | 691 | 5 | 0.96 |
| Sample 15 | 787 | 4.56 | 0.97 |

### Example 5

### Organosilica Material Syntheses Using Formula [Z¹Z²SiCH₂]₃ (Ia) and Trivalent Metal Oxides

### 5A. Synthesis Using [(EtO)₂SiCH₂]₃ and Aluminum-Tri-Sec-Butoxide

A solution with 39.6 g DI water (3410 mmol H₂O) and 31.15 g 30 wt % NH₄OH (265 mmol NH₄OH) was made. To the solution, 10 g (25 mmol) of [(EtO)₂SiCH₂]₃ (Reagent 1) and 0.37 g (1.5 mmol) aluminum-tri-sec-butoxide (Reagent 2) was added to produce a solution having the molar composition:

25.0[(EtO)₂SiCH₂]₃:1.5aluminum-tri-sec-butoxide:265OH:3410H₂O

which was stirred at 23-25° C. for 1 day. The Si/Al ratio between Reagent 1 and Reagent 2 was 50:1. The solution was transferred to an autoclave and aged at 90° C. for 1 day to produce a gel. The gel was dried in a vacuum at 120° C. for 1 day and Sample 22A was obtained. No surface directing agent or porogen were used.

The procedure was repeated except 1.845 g (7.5 mmol) aluminum-tri-sec-butoxide was added instead of 0.37 g (1.5 mmol) aluminum-tri-sec-butoxide to obtain Sample 22B. The Si/Al ratio between Reagent 1 and Reagent 2 was 10:1.

XRD Analysis

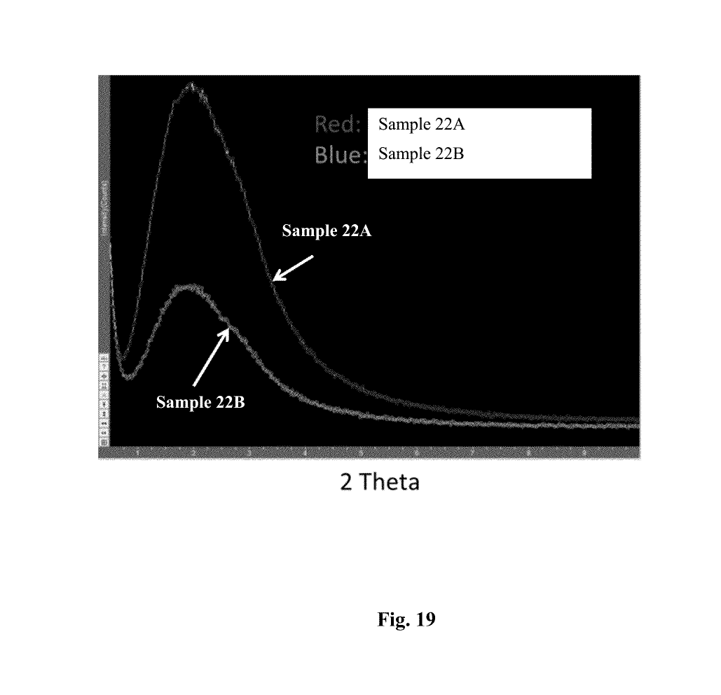

XRD was performed on Samples 22A and 22B. The XRD pattern of Samples 22A and 22B is shown in FIG. 19.

Nitrogen Adsorption/Desorption Analysis

Nitrogen adsorption/desorption analysis was performed on Samples 22A and 22B, and the results are provided in Table 10.

TABLE 10

| | Si/Al for | | | | Pore |
|---|---|---|---|---|---|
| | Reagent 1: | BET | SA (micro, | | diameter |
| Material | Reagent 2 | (m²/g) | m²/g) | V (cc/g) | (nm) |
| Sample 22A | 50 | 1273 | 646 | 0.679 | 2.13 |
| Sample 22B | 10 | 578 | 489 | 0.265 | 1.83 |

A highly porous material with more mesoporous structure was achieved when Si/Al ratio increases from 10 to 50.

SS-NMR-Analysis

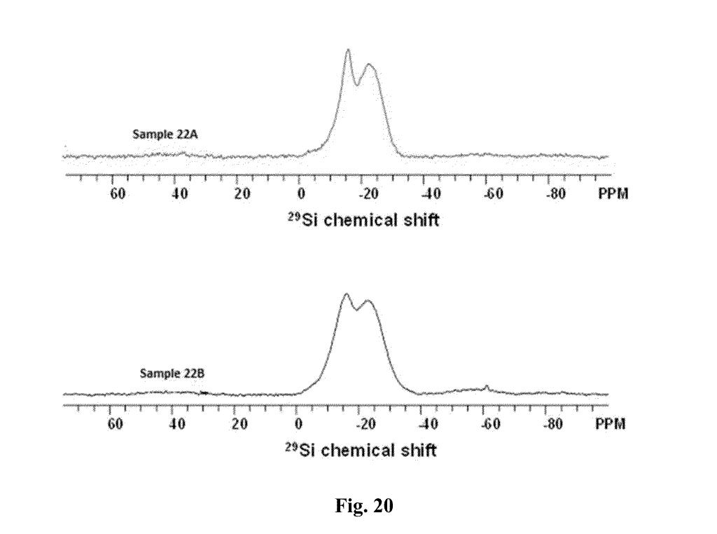

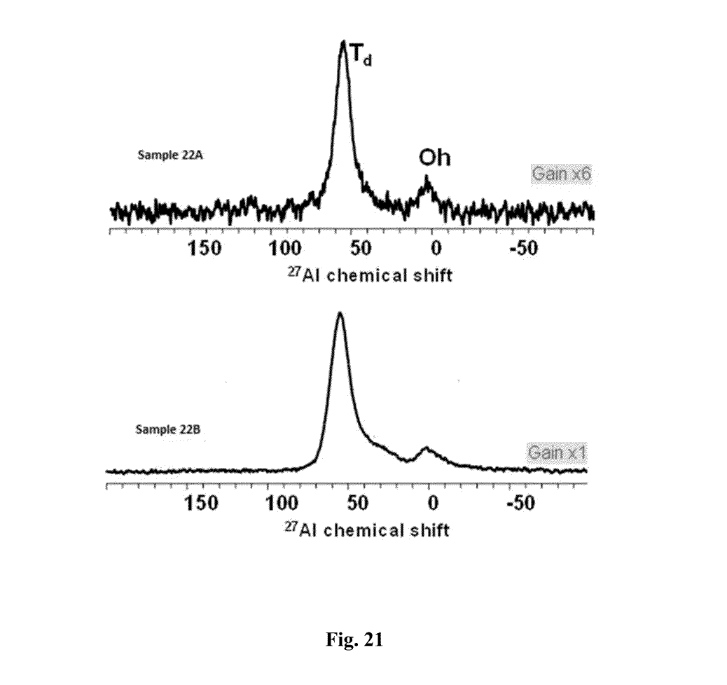

Samples 22A and 22B were characterized with ²⁹Si MAS NMR and ²⁷Al MAS NRM, as shown in FIGS. 20 and 21, respectively.

### Example 6

### pH, Gelation Time and Gelation Temperature Studies

### Example 6A

### Synthesis in Basic Solution (pH=8 to 13.4)

The effect of pH of the aqueous mixture during preparation of organosilica material was studied. Various organosilica materials were made with varying basic aqueous mixtures as follows:
1. Made a NH₄OH solution (about 14 g) with DI water with different pHs as shown in Table 11 below;
2. Added 1 g (2.5 mmol) reagent 1 [(EtO)₂SiCH₂]₃ into the above solution, kept stirring at 22° C. to 25° C. for 1 day;
3. Transferred the solution to an autoclave and aged at about 90° C. for 1 day; and
4. Dried the final product in an oven at about 120° C. under vacuum for 1 day.

TABLE 11

| | NH₄OH | NH₄OH | DI water | DI water | |
|---|---|---|---|---|---|
| Material | Amount (g) | Mol | Amount (g) | (mol) | pH |
| Sample A | 3.72 | 0.106 | 10.4 | 0.578 | 13.41 |
| Sample B | 1.86 | 0.053 | 12.3 | 0.682 | 12.55 |
| Sample C | 0.93 | 0.027 | 13.2 | 0.734 | 12.11 |
| Sample D | 0.3 | 0.0086 | 13.8 | 0.767 | 11.52 |
| Sample E | 0.09 | 0.0026 | 14.1 | 0.783 | 11.18 |
| Sample F | About | About | 14.3 | 0.794 | 10.64 |
| | 0.006 | 0.0002 | | | |
| Sample G | About | About | 14.3 | 0.794 | 10.17 |
| | 0.0004 | 0.00001 | | | |
| Sample H | About | About | 14.3 | 0.794 | 9.61 |
| | 0.00004 | 0.000001 | | | |
| Sample H1 | About | About | 14.2 | 0.789 | 8.0 |
| | 0.000004 | 0.0000001 | | | |

Nitrogen Adsorption/Desorption Analysis

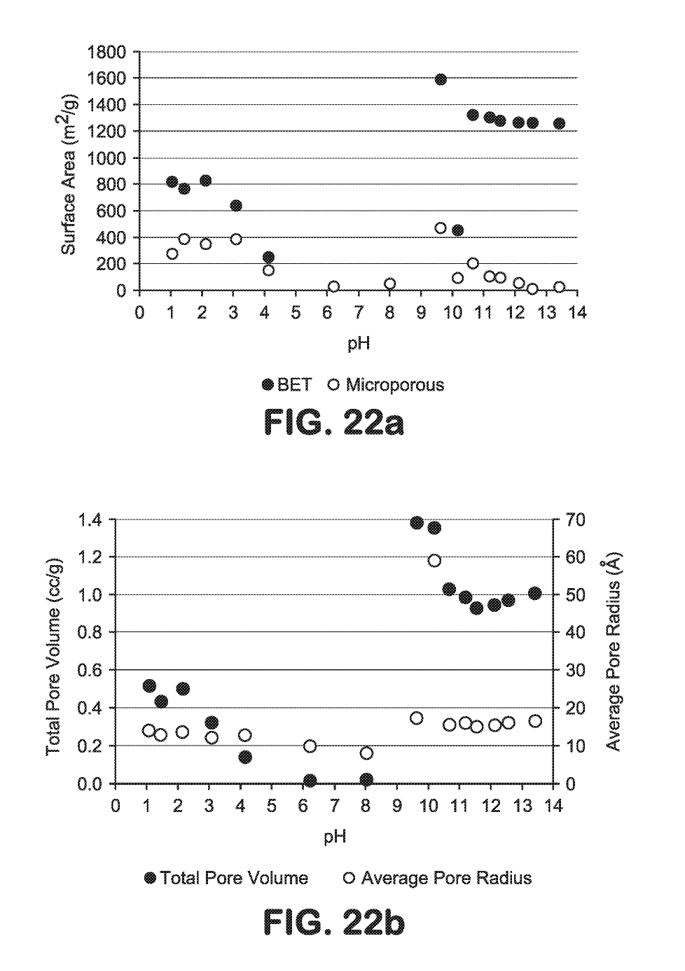

Nitrogen adsorption/desorption analysis was performed on Samples A-H1. The BET surface area, microporous surface area, average pore diameter, and pore volume obtained by the nitrogen adsorption/desorption analysis for Samples A-H1 are shown below in Table 12 and FIGS. 22a and 22b.

TABLE 12

| | | SA | | Pore diameter |
|---|---|---|---|---|
| Material | BET (m²/g) | (micro, m²/g) | V (cc/g) | (nm) |
| Sample A | 1266 | 27 | 1.011 | 3.32 |
| Sample B | 1263 | 14 | 0.971 | 3.2 |
| Sample C | 1270 | 56 | 0.946 | 3.1 |
| Sample D | 1285 | 99.7 | 0.928 | 3.04 |
| Sample E | 1308 | 107 | 0.988 | 3.2 |
| Sample F | 1325 | 205 | 1.03 | 3.12 |
| Sample G | 458 | 101 | 1.35 | 11.8 |
| Sample H | 1595 | 472 | 1.38 | 3.46 |
| Sample H1 | 52 | 56 | 0.021 | 1.65 |

### Example 6B

### Synthesis in Acidic Solution (pH=1.04 to 6.2)

Various organosilica materials were made with varying acidic aqueous mixtures as follows:
1. Make a HCl solution (about 14 g) with DI water with different pHs as shown in Table 13 below;
2. Added 1 g (2.5 mmol) reagent 1 (3-ring reagent) into the above solution, kept stirring at 22 to 25° C. for 1 day;
3. Transferred the solution to an autoclave and aged it at about 90° C. for 1 day; and
4. Dried the final product in an oven at about 120° C. under vacuum for 1 day.

TABLE 13

| | HCl | | DI water | DI water | |
|---|---|---|---|---|---|
| Material | Amount (g) | HCl Mol | Amount (g) | (mol) | pH |
| Sample H2 | About | About | 14.2 | 0.789 | 6.2 |
| | 0.00000397 | 0.00000011 | | | |
| Sample I | 0.0000397 | 0.0000011 | 14 | 0.778 | 4.12 |
| Sample J | 0.000397 | 0.000011 | 14 | 0.778 | 3.07 |
| Sample K | 0.00397 | 0.00011 | 14 | 0.778 | 2.11 |
| Sample L | 0.019 | 0.00052 | 14 | 0.778 | 1.43 |
| Sample M | 0.0466 | 0.00128 | 14 | 0.778 | 1.04 |

Nitrogen Adsorption/Desorption Analysis

Nitrogen adsorption/desorption analysis was performed on Samples H2-M. The BET surface area, microporous surface area average pore diameter, and pore volume obtained by the nitrogen adsorption/desorption analysis for Samples H2-M are shown below in Table 14 and FIGS. 22a and 22b.

TABLE 14

| | | SA | | Pore diameter |
|---|---|---|---|---|
| Material | BET (m²/g) | (micro, m²/g) | V (cc/g) | (nm) |
| Sample H | 228.4 | 33.8 | 0.014 | 2.03 |
| Sample I | 254 | 155 | 0.144 | 2.58 |
| Sample J | 642 | 389 | 0.325 | 2.44 |
| Sample K | 829 | 352 | 0.502 | 2.72 |
| Sample L | 770 | 388 | 0.436 | 2.58 |
| Sample M | 821 | 275 | 0.517 | 2.82 |

As shown in FIGS. 22a and 22b, adjusting the pH of the aqueous mixture can affect the BET surface area, microporous surface area and pore volume of the organosilica material made. The BET surface area generally increases with increased pH (i.e., as the aqueous mixture becomes more basic), while the microporous surface area generally decreases with increasing pH of the aqueous mixture (i.e., as the aqueous mixture becomes more basic). Thus, there may be a higher fraction of the total surface area being microporous at lower pH of the aqueous mixture (i.e. an acidic aqueous mixture).

### Example 6C

### Synthesis with Varying Aging Times at 90° C.

The effect of aging time during preparation of organosilica material was studied. Various organosilica materials were made with varying aging times as follows:
1. Made a NH₄OH solution (62.1 g, 30% wt) with 79.2 g DI water, pH=12.5;
2. Added 10 g (25 mmol) reagent 1 [(EtO)₂SiCH₂]₃ into the above solution, kept stirring at 22° C. to 25° C. for 1 day;
3. Transferred the solution to an autoclave and aged at about 90° C. for different times (0 to 144 hours) as shown in Table 15 below; and
4. Dried the final product in an oven at about 120° C. under vacuum for 1 day.

Nitrogen Adsorption/Desorption Analysis

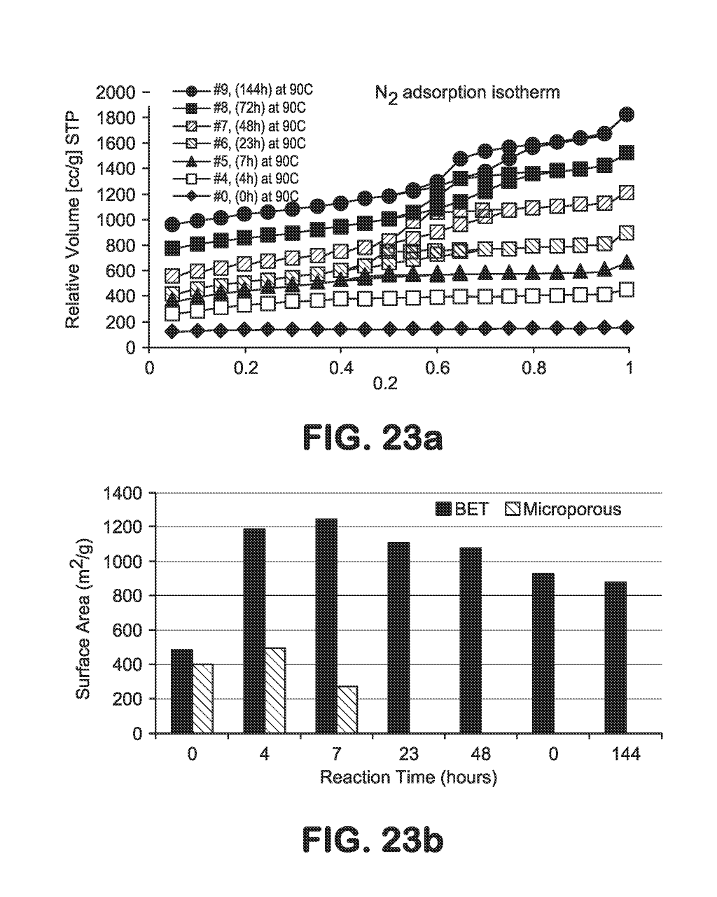

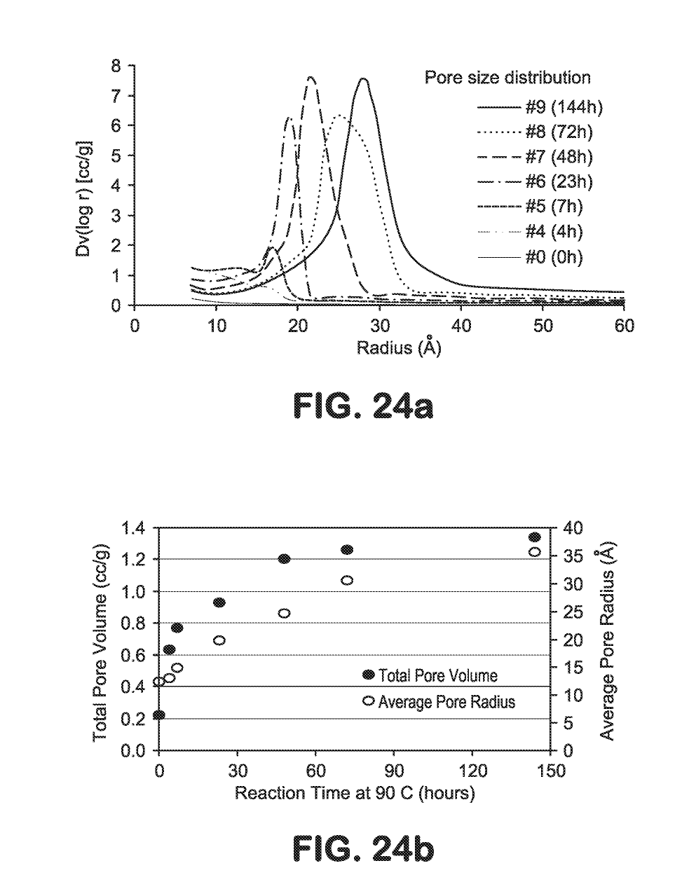

Nitrogen adsorption/desorption analysis was performed on Samples N-T. The BET surface area, microporous surface area, average pore radius, and pore volume obtained by the nitrogen adsorption/desorption analysis for Samples N-T are shown below in Table 15 and FIGS. 23a, 23b, 24a and 24b.

TABLE 15

| | Aging | | | | Pore |
|---|---|---|---|---|---|
| | Time | BET | SA | V | diameter |
| Material | (hr) | (m²/g) | (micro, m²/g) | (cc/g) | (nm) |
| Sample N | 0 | 485 | 398 | 0.227 | 2.48 |
| Sample O | 4 | 1191 | 500 | 0.639 | 2.6 |
| Sample P | 7 | 1247 | 276 | 0.772 | 2.98 |
| Sample Q | 23 | 1105 | 0 | 0.934 | 3.96 |
| Sample R | 48 | 1077 | 0 | 1.205 | 4.94 |
| Sample S | 72 | 929 | 0 | 1.262 | 6.12 |
| Sample T | 144 | 878 | 0 | 1.341 | 7.14 |

The organosilica material obtainable by the methods described herein may be advantageously obtainable at variable aging times and temperatures as discussed above. At early aging times, the nitrogen adsorption isotherm may exhibit complete reversibility whereby the adsorption and desorption legs of the isotherm are on top of each other. At some intermediate aging time a hysteresis may appear as an offset in the adsorption and desorption legs. The size of this offset may increase with increasing aging time to a point, after which it remains constant with increasing aging time. As shown in FIG. 23a, N₂ adsorption uptake capacity increases as aging time increases and the onset of an adsorption/desorption hysteresis loop was observed at 23 hours. Further, FIG. 23b shows that surface area was generally more microporous at shorter aging times but transitioned to primarily mesoporous as aging times increased. Additionally, average pore radius and pore volume generally increases as aging times increased, as shown in FIGS. 24a and 24b.

### Example 6D

### Synthesis with Varying Aging Times at 120° C.

The effect of aging time with an increased aging temperature during preparation of organosilica material was studied. Various organosilica materials were made with varying aging times at an increased temperature of 120° C. as follows:
1. Made a NH₄OH solution (31.05 g, 30% wt) with 39.6 g DI water, pH=12.5;
2. Added 5 g (12.5 mmol) reagent 1 [(EtO)₂SiCH₂]₃ into the above solution, kept stirring at 22° C. to 25° C. for 1 day;
3. Transferred the solution to an autoclave and aged at about 120° C. for different time (4 to 144 hours);
4. Dried the final product in an oven at about 120° C. under vacuum for 1 day.

Nitrogen Adsorption/Desorption Analysis

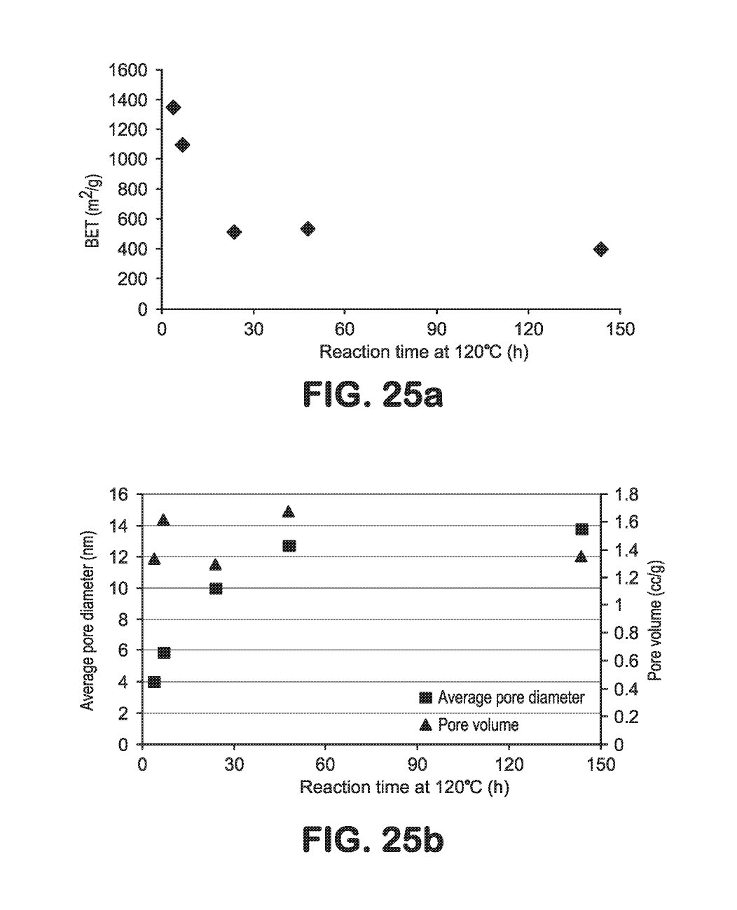

Nitrogen adsorption/desorption analysis was performed on Samples U-Y. The BET surface area, average pore diameter, and pore volume obtained by the nitrogen adsorption/desorption analysis for Samples U-Y are shown below in Table 16 and FIGS. 25a and 25b.

TABLE 16

| | Aging | | | | Pore |
|---|---|---|---|---|---|
| | Time | BET | SA | V | diameter |
| Material | (hr) | (m²/g) | (micro, m²/g) | (cc/g) | (nm) |
| Sample U | 4 | 1344 | 0 | 1.33 | 3.97 |
| Sample V | 7 | 1093 | 0 | 1.61 | 5.9 |
| Sample W | 24 | 509 | 0 | 1.29 | 10 |
| Sample X | 48 | 529 | 0 | 1.67 | 12.6 |
| Sample Y | 144 | 395 | 0 | 1.35 | 13.7 |

As shown in FIGS. 25a and 25b, increasing the aging temperature along with increased aging times accelerated in the changes in BET surface area, average pore diameter and pore volume observed when only the aging time was increased in Example 7C above.

SS-NMR-Analysis

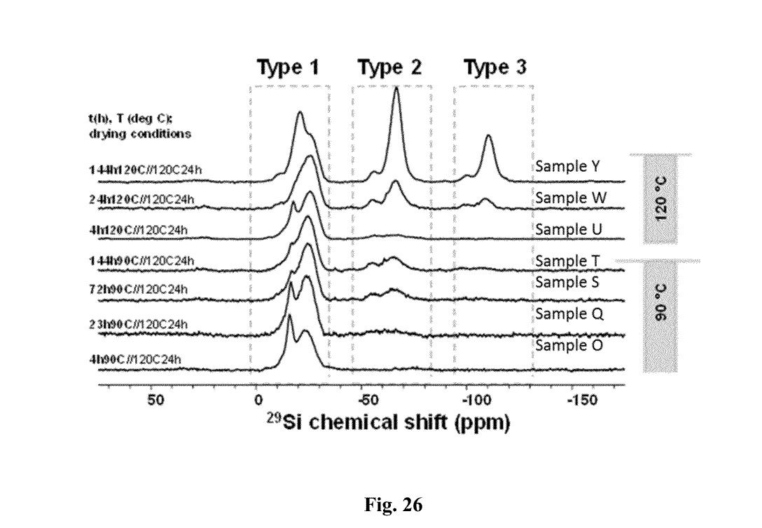

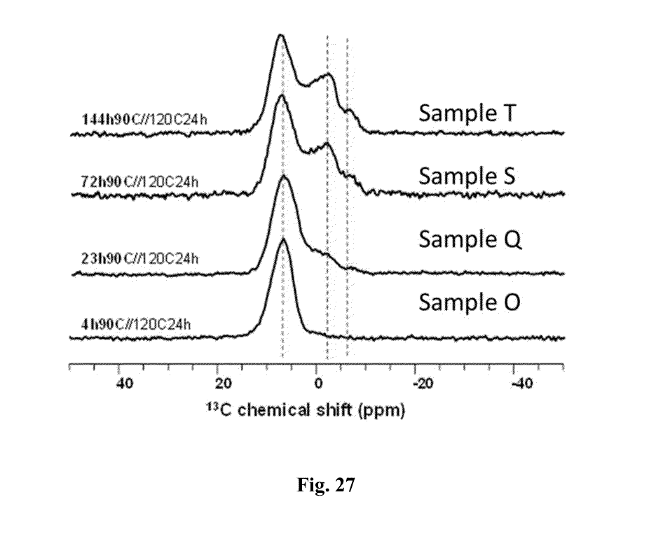

The materials in Table 15 and 16 were characterized with ²⁹Si MAS NMR and ¹³C CPMAS, as shown in FIGS. 26 and 27, respectively. The NMR data in FIG. 26 shows the generation of different types of Si species (designated as Type 1, Type 2 and Type 3). Depending on the pH, aging temperature and/or aging time, different proportions of these species were observed. The data indicates that there were changes in the structure, especially in the higher pH preparations. The Type 1 species are typically from Si species bonded to two carbon atoms and two oxygen atoms, which in turn are bonded to other Si or H atoms. Speciation within the Type 1 species is a result of microstructure. On the other hand, Type 2 species are typically from Si species bonded to three oxygen atoms and one carbon atom, which in turn are connected to other Si or H. Type 3 species arise from Si species bonded to four oxygen atoms, in turn bonded to other Si or H atoms.

FIG. 26 shows that Type 1 Si species are present initially and are joined by Types 2 and 3 at longer aging times (≧23 hrs at 90° C., and >4 hrs at 120° C.). Referring to FIGS. 12a and 12b, the transition from microporous to mesoporous at pH=12.5 and 90° C., is almost entirely complete at 23 hrs. aging, before the appearance of Types 2 and Type 3 Si species. The molecular changes observed in the NMR reflect changes in the Si environment under extended gelation times at pH=12.5.

In FIG. 27, the spectra show from a single band at the least severe condition (bottom) to at least three bands as the severity increases (top). The bands correspond to different types of carbon species, which indicate the structures at the least severe conditions are consistent with species such as Si—CH₂—Si and as the severity increases, structures consistent with Si—CH₃ groups are formed as evidenced by presence of structures consistent with Si—CH3 groups.

In sum, the surface are and porosity of the organosilica material may be adjusted by adjusting the pH of the aqueous mixture, the aging time and/or the aging temperature during the preparation process of the organosilica material.

### Example 7

### Hydrothermal Stability

Hydrothermal stability was tested for Sample 1A, Comparative Sample 2, and Sample 5. All the samples were treated in DI water at 140° C. for 7 days in an autoclave. The materials demonstrated significant hydrothermal stability and mesoporosity of the samples remained after the testing. A summary of the hydrothermal stability testing results and comparison to conventional mesoporous silicas is shown below in Table 17.

TABLE 17

| | | | Pore |
|---|---|---|---|
| | | | diameter |
| | BET (m²/g ) | V (cm³/g ) | (nm) |
| Comparative Sample 2 | 1256 | 0.88 | 3.02 |
| 140° C./H₂O/Comparative Sample 2 | 1358 | 1.02 | 3.06 |
| Sample 1A | 1409 | 0.91 | 3.18 |
| 140° C./H₂O/ Sample 1A | 1547 | 1.11 | 3.26 |
| Sample 5 | 1027 | 1.19 | 4.84 |
| 140° C./H₂O/ Sample 5 | 812 | 1.5 | 6.5 |

### Example 8

### CO₂ Isotherms

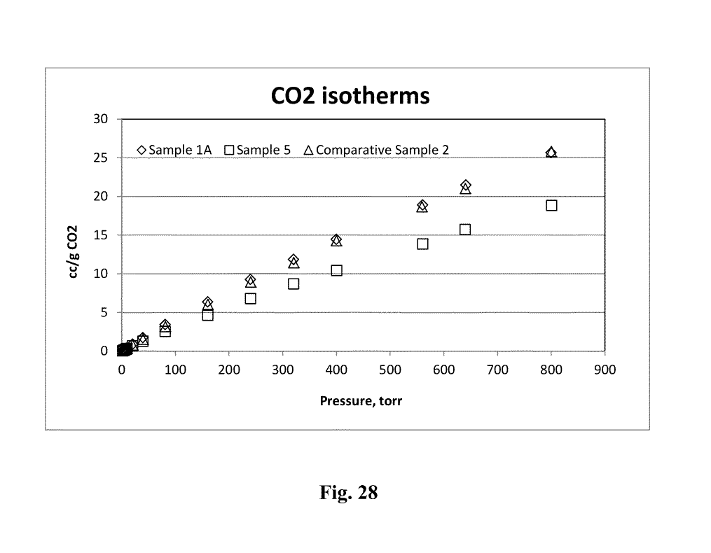

CO₂ adsorption isotherms were measured for Sample 1A, Comparative Sample 2, and Sample 5, as shown in FIG. 28. Sample 1A has similar CO₂ uptake compared to the Comparative Sample 2.

### Example 9

### Calcining Study

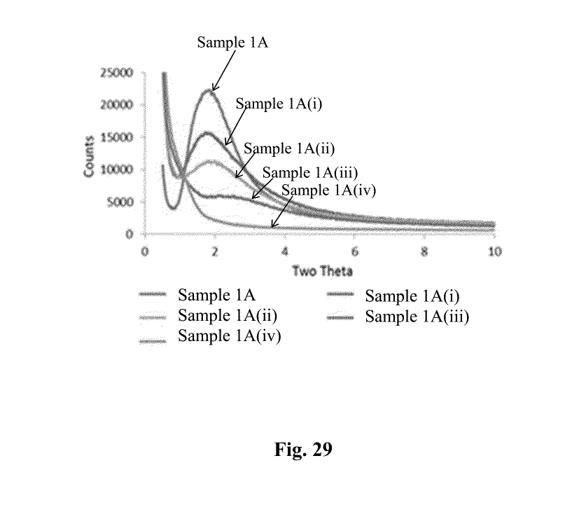

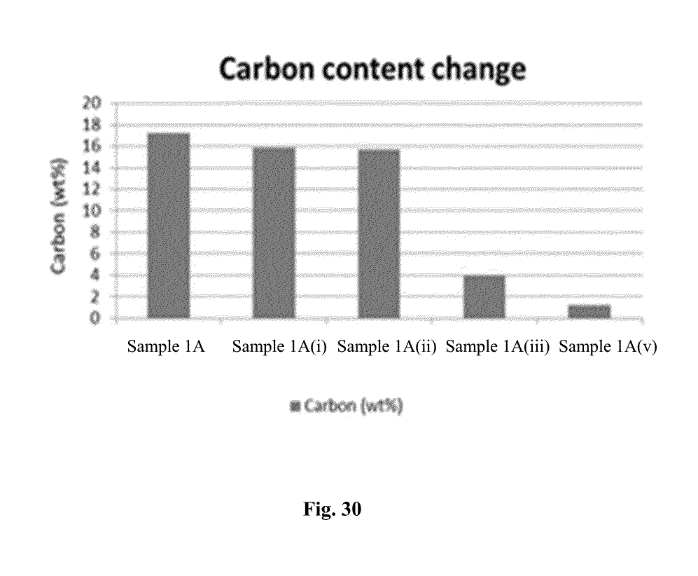

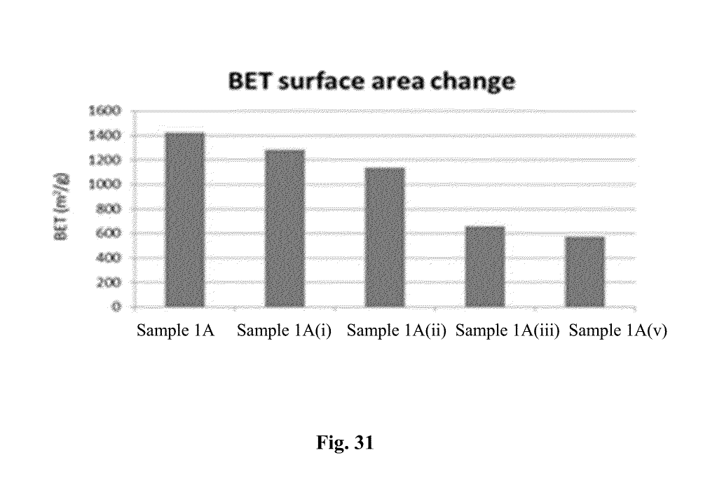

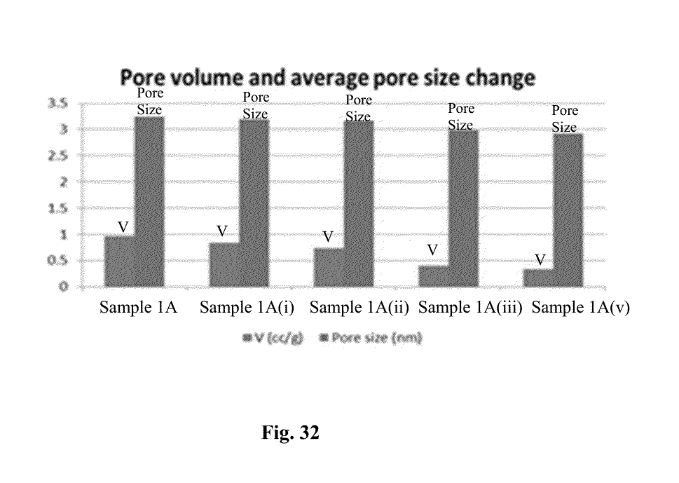

Sample 1A was calcined at temperatures of 300° C., 400° C., 500° C., and 600° C. in air to obtain Samples 1A(i), 1A(ii), 1A(iii) and 1A(iv), respectively. A comparison of the XRD patterns, the carbon content change, the BET surface area change, and the pore volume and average pore diameter change for Sample 1A and Samples 1A(i), 1A(ii), 1A(iii) and 1A(iv), are provided in FIGS. 29-32, respectively. As shown in FIGS. 29-32, after calcining at 500° C. Sample 1A(iii) still exhibited good mesoporosity (e.g., 3 nm pore diameter and over 600 m²/g surface area).

What is claimed is:

1. A method for preparing an organosilica material, the method comprising:
(a) providing an aqueous mixture that contains essentially no structure directing agent and/or porogen,
(b) adding at least one compound of Formula [Z¹Z²SiCH₂]₃ (Ia) into the aqueous mixture to form a solution, wherein each Z¹ represents a C₁-C₄ alkoxy group and each Z² represents a C₁-C₄ alkoxy group or a C₁-C₄ alkyl group;
(c) aging the solution to produce a pre-product; and
(d) drying the pre-product to obtain an organosilica material which is a polymer comprising independent siloxane units of Formula [Z³Z⁴SiCH₂]₃ (I), wherein each Z³ represents a hydroxyl group, a C₁-C₄ alkoxy group or an oxygen atom bonded to a silicon atom of another siloxane unit and each Z⁴ represents a hydroxyl group, a C₁-C₄ alkoxy group, a C₁-C₄ alkyl group, or an oxygen atom bonded to a silicon atom of another siloxane.

2. The method of claim 1, wherein each Z¹ represents a C₁-C₂ alkoxy group.

3. The method of claim 1, wherein each Z² represents a C₁-C₄ alkoxy group.

4. The method of claim 1, wherein each Z² represents a C₁-C₂ alkoxy group.

5. The method of claim 1, wherein the at least one compound of Formula (Ia) is 1,1,3,3,5,5-hexaethoxy-1,3,5-trisilacyclohexane.

6. The method of claim 1, wherein each Z³ represents a hydroxyl group, a C₁-C₂ alkoxy group, or an oxygen atom bonded to a silicon atom of another siloxane unit and each Z⁴ represent a hydroxyl group, a C₁-C₂ alkyl group, a C₁-C₂ alkoxy group, or an oxygen atom bonded to a silicon atom of another siloxane unit.

7. The method of claim 1, wherein each Z³ represents a hydroxyl group, ethoxy, or an oxygen atom bonded to a silicon atom of another siloxane and each Z⁴ represent a hydroxyl group, ethoxy, or an oxygen atom bonded to a silicon atom of another siloxane.

8. The method of claim 1, further comprising adding to the aqueous mixture at least one compound selected from the group consisting of
(i) a further compound of Formula (Ia)
(ii) a compound of Formula R¹OR²R³R⁴Si (II), wherein each R¹ represents a C₁-C₆ alkyl group, and R², R³ and R⁴ are each independently selected from the group consisting of a C₁-C₆ alkyl group, a C₁-C₆ alkoxy group, a nitrogen-containing C₁-C₁₀ alkyl group, a nitrogen-containing heteroaralkyl group, and a nitrogen-containing optionally substituted heterocycloalkyl group;
(iii) compound of Formula Z⁵Z⁶Z⁷Si—R—SiZ⁵Z⁶Z⁷ (III), wherein
each Z⁵ independently represents a C₁-C₄ alkoxy group;
each Z⁶ and Z⁷ independently represent a C₁-C₄ alkoxy group or a C₁-C₄ alkyl group; and
each R is selected from the group consisting a C₁-C₈ alkylene group, a C₂-C₈ alkenylene group, a C₂-C₈ alkynylene group, a nitrogen-containing C₁-C₁₀ alkylene group, an optionally substituted C₆-C₂₀ aralkyl and an optionally substituted C₄-C₂₀ heterocycloalkyl group;
(iv) a source of a trivalent metal oxide; and
(v) a combination thereof.

9. The method of claim 8, wherein the at least one compound is a further compound of Formula (Ia), wherein each Z¹ represents a C₁-C₂ alkoxy group and each Z² represent C₁-C₂ alkoxy group or a C₁-C₂ alkyl group.

10. The method of claim 9, wherein the compound of Formula (Ia) is 1,3,5-trimethyl-1,3,5-triethoxy-1,3,5-trisilacyclohexane.

11. The method of claim 8, wherein the at least one compound is a compound of Formula (II), wherein each R¹ represents a C₁-C₂ alkyl group and R², R³ and R⁴ are each independently a C₁-C₂ alkyl group, C₁-C₂ alkoxy group, a nitrogen-containing C₃-C₁₀ alkyl group, a nitrogen-containing C₄-C₁₀ heteroaralkyl group, or a nitrogen-containing optionally substituted C₄-C₁₀ heterocycloalkyl group.

12. The method of claim 11, wherein the compound of Formula (II) is selected from the group consisting of tetraethyl orthosilicate, methyltriethoxysilane, (N,N-dimethylaminopropyl)trimethoxysilane, N-(2-aminoethyl)-3-aminopropyltriethoxysilane, 4-methyl-1-(3-triethoxysilylpropyl)-piperazine, 4-(2-(triethoxysily)ethyl)pyridine, 1-(3-(triethoxysilyl)propyl)-4,5-dihydro-1H-imidazole, and (3-aminopropyl)triethoxysilane.

13. The method of claim 8, wherein the at least one compound is a compound of Formula (III), wherein each Z⁵ independently represents a C₁-C₂ alkoxy group; each Z⁶ and Z⁷ independently represent a C₁-C₂ alkoxy group, or a C₁-C₂ alkyl group; and each R is selected from the group consisting of a C₁-C₄ alkylene group, a C₂-C₄ alkenylene group, a C₂-C₄ alkynylene group, and a nitrogen-containing C₄-C₁₀ alkylene group.

14. The method of claim 13, wherein the compound of Formula (III) is selected from the group consisting of 1,2-bis(methyldiethoxysilyl)ethane, bis(triethoxysilyl)methane, 1,2-bis(triethoxysilyl)ethylene, N,N′-bis[(3-trimethoxysilyl)propyl]ethylenediamine, bis[(methyldiethoxysilyl)propyl]amine, and bis[(methyldimethoxysilyl)propyl]-N-methylamine.

15. The method of claim 8, wherein the at least one compound is a source of trivalent metal, wherein the source of trivalent metal is at least one of:
(i) a compound of Formula M¹(OZ⁸)₃ (IV), wherein M¹ represents a Group 13 metal and each Z⁸ independently represents a C₁-C₆ alkyl group; or
(ii) a compound of Formula (Z⁹O)₂M²-O—Si(OZ¹⁰)₃ (V), wherein M² represents a Group 13 metal and Z⁹ and Z¹⁰ each independently represent a C₁-C₆ alkyl group.

16. The method of claim 15, wherein the source of trivalent metal is a compound of Formula (IV), wherein M¹ is Al or B and each Z⁸ independently represents a C₁-C₄ alkyl group.

17. The method of claim 15, wherein the source of trivalent metal is a compound of Formula (V), wherein M² is Al or B; and each Z⁹ and each Z¹⁰ independently represent a C₁-C₄ alkyl group.

18. The method of claim 8, wherein the source of a trivalent metal oxide is selected from the group consisting of aluminum trimethoxide, aluminum triethoxide, aluminum isopropoxide, and aluminum-tri-sec-butoxide.

19. The method of claim 1, wherein the aqueous mixture comprises a base and has a pH from about 8 to about 14.

20. The method of claim 19, wherein the base is ammonium hydroxide or a metal hydroxide.

21. The method of claim 1, wherein the aqueous mixture comprises an acid and has a pH from about 0.01 to about 6.0.

22. The method of claim 21, wherein the acid is an inorganic acid.

23. The method of claim 22, wherein the inorganic acid is hydrochloric acid.

24. The method of claim 1, wherein the solution is aged in step (c) for up to 144 hours at a temperature of about 50° C. to about 200° C.

25. The method of claim 1, wherein the pre-product is dried at a temperature of about 70° C. to about 200° C.

26. The method of claim 1, wherein the organosilica material has an average pore diameter of about 2.0 nm to about 25.0 nm.

27. The method of claim 1, wherein the organosilica material has a total surface area of about 200 m²/g to about 2500 m²/g.

28. The method of claim 1, wherein the organosilica material has a pore volume of about 0.1 cm³/g to about 3.0 cm³/g.

29. The method of claim 19, wherein the organosilica material has one or more of the following:
(i) a total surface area of about 400 m²/g to about 1700 m²/g;
(ii) a microporous surface area of about 0 m²/g to about 600 m²/g; and
(iii) a pore volume of about 0.3 cm³/g to about 3.0 cm³/g.

30. The method of claim 21, wherein the organosilica material has one or more of the following:
(i) a total surface area of about 200 m²/g to about 1500 m²/g;
(ii) a microporous surface area of about 100 m²/g to about 900 m²/g; and
(iii) a pore volume of about 0.1 cm³/g to about 1.0 cm³/g.

31. The method of claim 1, wherein the solution is aged in step (c) for about 1 hour to about 7 hours at a temperature of about 80° C. to about 100° C. and the organosilica material has one or more of the following:
(i) a total surface area of about 400 m²/g to about 1300 m²/g;
(ii) a microporous surface area of about 200 m²/g to about 600 m²/g;
(iii) a pore volume of about 0.2 cm³/g to about 0.8 cm³/g; and
(iv) an average pore radius of about 1.0 nm to about 1.5 nm.

32. The method of claim 1, wherein the solution is aged in step (c) for greater than about 7 hours to about 150 hours at a temperature of about 80° C. to about 100° C. and the organosilica material has one or more of the following:
(i) a total surface area of about 800 m²/g to about 1200 m²/g;
(ii) a pore volume of greater than about 0.8 cm³/g to about 1.4 cm³/g; and
(iii) an average pore radius of greater than about 1.5 nm to about 4.0 nm.

33. The method of claim 1, wherein the solution is aged in step (c) for about 1 hour to about 7 hours at a temperature of about 110° C. to about 130° C. and the organosilica material has one or more of the following:
(i) a pore volume of about 1.4 cm³/g to about 1.7 cm³/g; and
(ii) an average pore diameter of about 4.0 nm to about 6.0 nm.

34. The method of claim 1, wherein the solution is aged in step (c) for greater than about 7 hours to about 150 hours at a temperature of about 110° C. to about 130° C. and the organosilica material has one or more of the following:
(i) a pore volume of about 1.2 cm³/g to about 1.8 cm³/g; and
(ii) an average pore diameter of about 10.0 nm to about 14 nm.

35. The method of claim 1, further comprising incorporating at least one catalytic metal within the pores of the organosilica material.

36. The method of claim 35, wherein the catalytic metals is selected from the group consisting of a Group 6 element, a Group 8 element, a Group 9 element, a Group 10 element and a combination thereof.

37. An organosilica material made according to the method of claim 1.

38. A catalyst material comprising the organosilica material of claim 37 and optionally, a binder.

39. A method for preparing an organosilica material, the method comprising:
(a) adding a compound corresponding in structure to Formula (Ia) 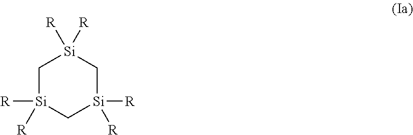
wherein each R is independently selected from the group consisting of a C₁-C₂ alkoxy and a C₁-C₂ alkyl into an aqueous mixture to form a solution;
(b) aging the solution to produce a gel; and
(c) drying the gel to obtain the organosilica material having an X-ray diffraction spectrum exhibiting substantially no peaks above 6 degrees 2θ; and wherein the method is performed using substantially no structure directing agent.

40. The method of claim 39, wherein each R is ethoxy.

41. The method of claim 39, wherein the organosilica material is made using substantially no added porogen.

42. The method of claim 39, wherein the organosilica material comprises units independently corresponding in structure to Formula (I) 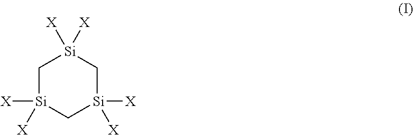
wherein each X is independently selected from the group consisting of a C₁-C₂ alkoxy, a C₁-C₂ alkyl and a hydroxyl, wherein the units are connected via at least one Si—O—Si linkage.

43. The method of claim 39, further comprising adding a reactant selected from the group consisting of tetraethyl orthosilicate, 1,2-bis(methyldiethoxysilyl)ethane, bis(triethoxysilyl)methane, 1,2-bis(triethoxysilyl)ethylene, 1,3,5-trimethyl-1,3,5-triethoxy-1,3,5-trisilacyclohexane, methyltriethoxysilane, and a combination thereof into the aqueous mixture to form the solution.
